SEQUENCE LISTING (151 sequences)

SEQ ID NO: 1 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDALRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 2 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 3 - protein, 1816 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 4 - protein, 992 aa, Artificial Sequence
  G domain of human LAMA4
SKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGRVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCP

SEQ ID NO: 5 - protein, 206 aa, Artificial Sequence
  LG1 module of G domain of human LAMA4
SKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPC

SEQ ID NO: 6 - protein, 180 aa, Artificial Sequence
  LG2 module of G domain of human LAMA4
ASYFFDGSGYAVVRDITRRGKFGQVTRFDIEVRTPADNGLILLMVNGSMFFRLEMRNGYLHVFYDFGFSSGRVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGC

SEQ ID NO: 7 - protein, 169 aa, Artificial Sequence
  LG3 module of G domain of human LAMA4
SRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYEC

SEQ ID NO: 8 - protein, 601 aa, Artificial Sequence
  LG1-3 modules of G domain of human LAMA4
SKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGRVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKG

SEQ ID NO: 9 - protein, 171 aa, Artificial Sequence
  LG4 module of G domain of human LAMA4
YQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPC

SEQ ID NO: 10 - protein, 174 aa, Artificial Sequence
  LG5 module of G domain of human LAMA4
TGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSC

SEQ ID NO: 11 - protein, 369 aa, Artificial Sequence
  LG4-5 modules of G domain of human LAMA4
RNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCP

SEQ ID NO: 12 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 13 - protein, 1130 aa, Homo sapiens
MAAAGQLCLLYLSAGLLSRLGAAFNLDTREDNVIRKYGDPGSLFGFSLAMHWQLQPEDKRLLLVGAPRAEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGLLFLTSVSYTDPDQFVYKTRPPREQPDTFPDVMMNSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPDLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRSRYDDSVPRYHAVRIRKEEREIKDEKYIDNLEKKQWITKWNENESYS

SEQ ID NO: 14 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 15 - protein, 116 aa, Artificial Sequence
  19C12 mature heavy chain variable region
QVQLQQSGAELVKPGASVKISCKASGYAFSTYWMNWVKQRPGEGLEWIGQIYPGDGDTNYNGKFKGKATLTADKSSSTAYMQLSSLTSEDSAVYFCARSDGYYDYWGQGTTLTVSS

SEQ ID NO: 16 - protein, 112 aa, Artificial Sequence
  19C12 mature light chain variable region
NIVLTQSPASLAVSLGQRATISCRASESVDSYGTSFMHWYQQKPGQPPKLLICLASSLESGVPARFSGSGSRTDFTLTIDPVEADDAATYYCQQNNEDPPTFGAGTKLELKR

SEQ ID NO: 17 - protein, 19 aa, Artificial Sequence
  19C12 heavy chain variable region signal peptide
MEWPLIFLFLLSGTAGVQS

SEQ ID NO: 18 - protein, 20 aa, Artificial Sequence
  19C12 light chain variable region signal peptide
METDTLLLWVLLLWVPGSTG

SEQ ID NO: 19 - protein, 5 aa, Artificial Sequence
  19C12 CDR-H1
TYWMN

SEQ ID NO: 20 - protein, 17 aa, Artificial Sequence
  19C12 CDR-H2
QIYPGDGDTNYNGKFKG

SEQ ID NO: 21 - protein, 7 aa, Artificial Sequence
  19C12 CDR-H3
SDGYYDY

SEQ ID NO: 22 - protein, 15 aa, Artificial Sequence
  19C12 CDR-L1
RASESVDSYGTSFMH

SEQ ID NO: 23 - protein, 7 aa, Artificial Sequence
  19C12 CDR-L2
LASSLES

SEQ ID NO: 24 - protein, 9 aa, Artificial Sequence
  19C12 CDR-L3
QQNNEDPPT

SEQ ID NO: 25 - protein, 121 aa, Artificial Sequence
  1C1 mature heavy chain variable region, version 1
QVQLQQSGAELARPGASVKLSCKASGYTFTNYGISWVKQRTGQGLEWIGAIYPRSGNTYYNEKFKGKATLTADKSSSTAYMELRSLTSEDSAVYFCARERDYGSSYALDYWGQGTSVTVSS

SEQ ID NO: 26 - protein, 108 aa, Artificial Sequence
  1C1 mature light chain variable region
DIVMTQSQKFMSTSVGDRVSVTCKASHNVGTNVAWYQQKPGQSPKPLIYSASYRYSGVPDRFTGSGSGTDFTLTISNVQSEDLADYFCQQFNNYPYTFGGGTKLEIKR

SEQ ID NO: 27 - protein, 19 aa, Artificial Sequence
  1C1 heavy chain variable region signal peptide, version 1
MGWSWIFIFILSGTAGVQS

SEQ ID NO: 28 - protein, 20 aa, Artificial Sequence
  1C1 light chain variable region signal peptide
MESQTQVFVYMLLWLSGVDG

SEQ ID NO: 29 - protein, 5 aa, Artificial Sequence
  1C1 CDR-H1, version 1
NYGIS

SEQ ID NO: 30 - protein, 17 aa, Artificial Sequence
  1C1 CDR-H2, version 1
AIYPRSGNTYYNEKFKG

SEQ ID NO: 31 - protein, 12 aa, Artificial Sequence
  1C1 CDR-H3, version 1
ERDYGSSYALDY

SEQ ID NO: 32 - protein, 11 aa, Artificial Sequence
  1C1 CDR-L1
KASHNVGTNVA

SEQ ID NO: 33 - protein, 7 aa, Artificial Sequence
  1C1 CDR-L2
SASYRYS

SEQ ID NO: 34 - protein, 9 aa, Artificial Sequence
  1C1 CDR-L3
QQFNNYPYT

SEQ ID NO: 35 - protein, 124 aa, Artificial Sequence
  5A12 mature heavy chain variable region, v1
QVQLQQPGAELVKPGASVKLSCKASGYTFTSYWMHWVKQRPGRGLEWIGRIDPNSGGTKHNEKFKSKATLTVDKPSSTAYMQLSSLTSEDSAVYYCAGSPSYYDYALHYAMDYWGQGTSVTVSS

SEQ ID NO: 36 - protein, 116 aa, Artificial Sequence
  5A12 mature heavy chain variable region, v2
QVQLQQSGAELVKPGASVKISCKASGYAFSTYWMNWVKQKPGKGLEWIGQIYPGDGDTNYNGKFKGKATLTAXKSSSTAYMQLXSLTSEDSAVYFCARSDGYYDYWGQGTTLTVSS

SEQ ID NO: 37 - protein, 112 aa, Artificial Sequence
  5A12 mature light chain variable region
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPRTFGGGTKLEIKR

SEQ ID NO: 38 - protein, 19 aa, Artificial Sequence
  5A12 heavy chain variable region signal peptide, v1
MGWSCIMLFLAATATGVHS

SEQ ID NO: 39 - protein, 19 aa, Artificial Sequence
  5A12 heavy chain variable region signal peptide, v2
MEWPLIFLFLLSGTAGVQS

SEQ ID NO: 40 - protein, 20 aa, Artificial Sequence
  5A12 light chain variable region signal peptide
METDTLLLWVLLLWVPGSTG

SEQ ID NO: 41 - protein, 5 aa, Artificial Sequence
  5A12 CDR-H1, v1
SYWMH

SEQ ID NO: 42 - protein, 17 aa, Artificial Sequence
  5A12 CDR-H2, v1
RIDPNSGGTKHNEKFKS

SEQ ID NO: 43 - protein, 15 aa, Artificial Sequence
  5A12 CDR-H3, v1
SPSYYDYALHYAMDY

SEQ ID NO: 44 - protein, 5 aa, Artificial Sequence
  5A12 CDR-H1, v2
TYWMN

SEQ ID NO: 45 - protein, 17 aa, Artificial Sequence
  5A12 CDR-H2, v2
QIYPGDGDTNYNGKFKG

SEQ ID NO: 46 - protein, 7 aa, Artificial Sequence
  5A12 CDR-H3, v2
SDGYYDY

SEQ ID NO: 47 - protein, 15 aa, Artificial Sequence
  5A12 CDR-L1
KASQSVDYDGDSYMN

SEQ ID NO: 48 - protein, 7 aa, Artificial Sequence
  5A12 CDR-L2
AASNLES

SEQ ID NO: 49 - protein, 9 aa, Artificial Sequence
  5A12 CDR-L3
QQSNEDPRT

SEQ ID NO: 50 - protein, 124 aa, Artificial Sequence; identical to SEQ ID NO: 35
  5B5 mature heavy chain variable region

SEQ ID NO: 51 - protein, 112 aa, Artificial Sequence; identical to SEQ ID NO: 37
  5B5 mature light chain variable region

SEQ ID NO: 52 - protein, 19 aa, Artificial Sequence
  5B5 heavy chain variable region signal peptide
MGWSCIMLFLAATATGVHS

SEQ ID NO: 53 - protein, 20 aa, Artificial Sequence
  5B5 light chain variable region signal peptide
METDTILLWVLLLWVPGSTG

SEQ ID NO: 54 - protein, 5 aa, Artificial Sequence
  5B5 CDR-H1
SYWMH

SEQ ID NO: 55 - protein, 17 aa, Artificial Sequence
  5B5 CDR-H2
RIDPNSGGTKHNEKFKS

SEQ ID NO: 56 - protein, 15 aa, Artificial Sequence
  5B5 CDR-H3
SPSYYDYALHYAMDY

SEQ ID NO: 57 - protein, 15 aa, Artificial Sequence
  5B5 CDR-L1
KASQSVDYDGDSYMN

SEQ ID NO: 58 - protein, 7 aa, Artificial Sequence
  5B5 CDR-L2
AASNLES

SEQ ID NO: 59 - protein, 9 aa, Artificial Sequence
  5B5 CDR-L3
QQSNEDPRT

SEQ ID NO: 60 - protein, 119 aa, Artificial Sequence
  12D3 mature heavy chain variable region, v1
QVQLQQPGAELVKPGASVKLSCKASGYTFTSYWMHWVKQRPGQGLEWIGMIHPHSGSTNYNEKFKSKATLTVDKSSSTAYMQLSSLTSEDSAVYYCARQLRLLYYFDYWGQGTTLTVSS

SEQ ID NO: 61 - protein, 121 aa, Artificial Sequence; identical to SEQ ID NO: 25
  12D3 mature heavy chain variable region, v2

SEQ ID NO: 62 - protein, 114 aa, Artificial Sequence
  12D3 mature light chain variable region
DIVMTQSPSSLTVTAGEKVTMSCKSSQSLLNSGNQKNYLTWYQQKPGQPPKLLIYWASTRESGVPDRFAGSGSGTDFTLTISSVQAEDLAVYYCQNDYDYPLTFGAGTKLELKR

SEQ ID NO: 63 - protein, 19 aa, Artificial Sequence
  12D3 heavy chain variable region signal peptide, v1
MGWSYIILFLVATATGVHS

SEQ ID NO: 64 - protein, 19 aa, Artificial Sequence
  12D3 heavy chain variable region signal peptide, v2
MEWIWIFLFILSGTAGVQS

SEQ ID NO: 65 - protein, 20 aa, Artificial Sequence
  12D3 light chain variable region signal peptide
MESQTQVLMSLLFWVSGTRG

SEQ ID NO: 66 - protein, 5 aa, Artificial Sequence
  12D3 CDR-H1, v1
SYWMH

SEQ ID NO: 67 - protein, 17 aa, Artificial Sequence
  12D3 CDR-H2, v1
MIHPHSGSTNYNEKFKS

SEQ ID NO: 68 - protein, 10 aa, Artificial Sequence
  12D3 CDR-H3, v1
QLRLLYYFDY

SEQ ID NO: 69 - protein, 5 aa, Artificial Sequence
  12D3 CDR-H1, v2
NYGIS

SEQ ID NO: 70 - protein, 17 aa, Artificial Sequence
  12D3 CDR-H2, v2
AIYPRSGNTYYNEKFKG

SEQ ID NO: 71 - protein, 12 aa, Artificial Sequence
  12D3 CDR-H3, v2
ERDYGSSYALDY

SEQ ID NO: 72 - protein, 17 aa, Artificial Sequence
  12D3 CDR-L1
KSSQSLLNSGNQKNYLT

SEQ ID NO: 73 - protein, 7 aa, Artificial Sequence
  12D3 CDR-L2
AASNLES

SEQ ID NO: 74 - protein, 9 aa, Artificial Sequence
  12D3 CDR-L3
QQSNEDPRT

SEQ ID NO: 75 - protein, 119 aa, Artificial Sequence
  Human VH acceptor FR
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARDTHSWFAFDIWGQGTMVTVSS

SEQ ID NO: 76 - protein, 114 aa, Artificial Sequence
  Human VL acceptor FR, v1
DIVMTQSPLSLAVSLGERATINCKSSQSVLFSSNNKNYLAWFQQKPGQPPKLLISWASTRESGVPDRFSGSGSGTDFTLTIDNLQAGDVALYYCQQYFSSPCTFGQGTKLEIKR

SEQ ID NO: 77 - protein, 114 aa, Artificial Sequence
  Human VL acceptor FR, v2
EIVLTQSPDSLAVSLGERATINCKSSQSILYNTNSKNHLVWYQQKPGQPPKVLIYWASTRGSGVPARFSGSGSETDFTLTISSLQAEDAAVYYCQQNYRTPYTFGQGTKLEIKR

SEQ ID NO: 78 - protein, 116 aa, Artificial Sequence
  Synthesized
QVQLQQSGAEVKKPGSSVKVSCKASGGTFSTYWMNWVRQAPGQGLEWMGQIYPGDGDTNYNGKFKGRVTITADKSTSTAYMELSSLRSEDTAVYYCARSDGYYDYWGQGTMVTVSS

SEQ ID NO: 79 - protein, 112 aa, Artificial Sequence
  Synthesized
DIVLTQSPLSLAVSLGERATINCRASESVDSYGTSFMHWYQQKPGQPPKLLISLASSLESGVPARFSGSGSGTDFTLTISSLQAEDVALYYCQQNNEDPPTFGQGTKLEIKR

SEQ ID NO: 80 - protein, 116 aa, Artificial Sequence
  Synthesized
QVQLQQSGAELVKPGASVKISCKASGYAFSTYWMNWVKQAPGEGLEWIGQIYPGDGDTNYNGKFKGRVTLTADKSTSTAYMELSSLRSEDTAVYFCARSDGYYDYWGQGTTVTVSS

SEQ ID NO: 81 - protein, 116 aa, Artificial Sequence
  Synthesized
QVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSS

SEQ ID NO: 82 - protein, 116 aa, Artificial Sequence
  Synthesized
EVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSS

SEQ ID NO: 83 - protein, 112 aa, Artificial Sequence
  Synthesized
NIVLTQSPASLAVSLGERATISCRASESVDSYGTSFMHWYQQKPGQPPKLLICLASSLESGVPDRFSGSGSRTDFTLTISSLQAEDVATYYCQQNNEDPPTFGQGTKLEIKR

SEQ ID NO: 84 - protein, 112 aa, Artificial Sequence
  Synthesized
NIVLTQSPASLAVSLGERATISCRASESVDSYGTSFMHWYQQKPGQPPKLLISLASSLESGVPDRFSGSGSGTDFTLTISSLQAEDVATYYCQQNNEDPPTFGQGTKLEIKR

SEQ ID NO: 85 - protein, 112 aa, Artificial Sequence
  Synthesized
NIVLTQSPASLAVSLGERATISCRASESVDSYGTSFMHWYQQKPGQPPKLLICLASSLESGVPARFSGSGSRTDFTLTIDPVEAEDAATYYCQQNNEDPPTFGAGTKLEIKR

SEQ ID NO: 86 - protein, 112 aa, Artificial Sequence
  Synthesized
NIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKR

SEQ ID NO: 87 - protein, 112 aa, Artificial Sequence
  Synthesized
DIVLTQSPASLAVSLGERATISCRASESVDSYGTSFMHWYQQKPGQPPKLLISLASSLESGVPARFSGSGSGTDFTLTISPLQAEDVATYYCQQNNEDPPTFGQGTKLEIKR

SEQ ID NO: 88 - protein, 112 aa, Artificial Sequence
  Synthesized
DIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKR

SEQ ID NO: 89 - protein, 330 aa, Artificial Sequence
  Exemplary human IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 106 aa, Artificial Sequence
  Exemplary human kappa light chain constant region without a N-terminal arginine
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 348 bp, Artificial Sequence
  19C12 mature heavy chain variable region
caggttcagctgcagcagtctggggctgagctggtgaagcctggggcctcagtgaagatttcctgcaaagcttctggctacgcattcagtacctactggatgaactgggtgaagcagaggcctggagagggtcttgagtggattggacagatttatcctggagatggtgatactaactacaacggaaagttcaagggcaaggccacactgactgcagacaagtcctccagcacagcctacatgcagctcagcagcctgacctctgaggactctgcggtctatttctgtgcaagatcagatggttactacgactactggggccaaggcaccactctcacagtctcctca

SEQ ID NO: 92 - DNA, 336 bp, Artificial Sequence
  19C12 mature light chain variable region
aacattgtgctgacccaatctccagcttctttggctgtgtctctagggcagagggccaccatatcctgcagagccagtgaaagtgttgatagttatggcactagttttatgcactggtaccagcagaaaccaggacagccacccaaactcctcatctgtcttgcatccagcctagaatctggggtccctgccaggttcagtggcagtgggtctaggacagacttcaccctcaccattgatcctgtggaggctgatgatgctgcaacctattactgtcagcaaaataatgaggatcctcctacgttcggtgctgggaccaagctggagctgaaacgt

SEQ ID NO: 93 - DNA, 57 bp, Artificial Sequence
  19C12 heavy chain variable region signal peptide
atggaatggcctttgatctttctcttcctcctgtcaggaactgcaggtgtccaatcc

SEQ ID NO: 94 - DNA, 60 bp, Artificial Sequence
  19C12 light chain variable region signal peptide
atggagacagacacactcctgctatgggtgctgctgctctgggttccaggttccacaggt

SEQ ID NO: 95 - DNA, 363 bp, Artificial Sequence
  1C1 mature heavy chain variable region, version 1
caggttcagctgcagcagtctggagctgaactggcgaggcctggggcttcagtgaagctgtcctgcaaggcttctggctacaccttcacaaactatggaataagctgggtgaagcagagaactggacagggccttgagtggattggagcgatttatcctagaagtggtaatacttactacaatgagaagttcaagggcaaggccacactgactgcagacaaatcctccagcacagcgtacatggagctccgcagcctgacatctgaggactctgcggtctatttctgtgcaagagaaagggactacggtagtagctacgctctggactactggggtcaaggaacctcagtcaccgtctcctca

SEQ ID NO: 96 - DNA, 324 bp, Artificial Sequence
  1C1 mature light chain variable region
gacattgtgatgacccagtctcaaaaattcatgtccacatcagtaggagacagggtcagcgtcacctgcaaggccagtcacaatgtgggtactaatgtagcctggtatcaacagaaaccagggcaatctcctaaaccactgatttactcggcatcctaccgttacagtggagtccctgatcgcttcacaggcagtggatctgggacagatttcactctcaccatcagcaatgtgcagtctgaagacttggcagactatttctgtcagcaatttaacaactatccgtacacgttcggaggggggaccaagctggaaataaaacgg

SEQ ID NO: 97 - DNA, 57 bp, Artificial Sequence
  1C1 heavy chain variable region signal peptide, version 1
atgggatggagctggatctttatcttcatcctgtcaggaactgcaggtgtccaatcc

SEQ ID NO: 98 - DNA, 60 bp, Artificial Sequence
  1C1 light chain variable region signal peptide
atggagtcacagactcaggtctttgtatacatgttgctgtggttgtctggtgttgatgga

SEQ ID NO: 99 - DNA, 372 bp, Artificial Sequence
  5A12 mature heavy chain variable region, v1
caggtccaactgcagcagcctggggctgagcttgtgaagcctggggcttcagtgaagctgtcctgcaaggcttctggctacaccttcaccagctactggatgcactgggtgaagcagaggcctggacgaggccttgagtggattggaaggattgatcctaatagtggtggtactaagcacaatgagaagttcaagagcaaggccacactgactgtagacaaaccctccagcacagcctacatgcagctcagcagcctgacatctgaggactctgcggtctattattgtgcaggaagtccctcttactatgattacgcccttcactatgctatggactactggggtcaaggaacctcagtcaccgtctcctca

SEQ ID NO: 100 - DNA, 348 bp, Artificial Sequence; identical to SEQ ID NO: 91
  5A12 mature heavy chain variable region, v2

SEQ ID NO: 101 - DNA, 336 bp, Artificial Sequence
  5A12 mature light chain variable region
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcaacaaagtaatgaggatcctcggacgttcggtggaggcaccaaactggaaatcaaacgt

SEQ ID NO: 102 - DNA, 57 bp, Artificial Sequence
  5A12 heavy chain variable region signal peptide, v1
atgggatggagctgtatcatgctcttcttggcagcaacagctacaggtgtccactcc

SEQ ID NO: 103 - DNA, 57 bp, Artificial Sequence; identical to SEQ ID NO: 93
  5A12 heavy chain variable region signal peptide, v2

SEQ ID NO: 104 - DNA, 60 bp, Artificial Sequence
  5A12 light chain variable region signal peptide
atggagacagacacactcctgctatgggtgctgctgctctgggttccaggctccactggt

SEQ ID NO: 105 - DNA, 372 bp, Artificial Sequence; identical to SEQ ID NO: 99
  5B5 mature heavy chain variable region

SEQ ID NO: 106 - DNA, 336 bp, Artificial Sequence
  5B5 mature light chain variable region
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcaacaaagtaatgaggatcctcggacgttcggtggaggcaccaaactggaaatcaaacgg

SEQ ID NO: 107 - DNA, 57 bp, Artificial Sequence; identical to SEQ ID NO: 102
  5B5 heavy chain variable region signal peptide

SEQ ID NO: 108 - DNA, 60 bp, Artificial Sequence
  5B5 light chain variable region signal peptide
atggagacagacacaatcctgctatgggtgctgctgctctgggttccaggctccactggt

SEQ ID NO: 109 - DNA, 357 bp, Artificial Sequence
  12D3 mature heavy chain variable region, v1
caggtccaactgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctcatagtggtagtactaactacaatgagaagttcaagagcaaggccacactgactgtagacaagtcctccagcacagcctacatgcaactcagcagcctgacatctgaggactctgcggtctattactgtgcaagacagctcaggctactctactactttgactactggggccaaggcaccactctcacagtctcctca

SEQ ID NO: 110 - DNA, 363 bp, Artificial Sequence; identical to SEQ ID NO: 95
  12D3 mature heavy chain variable region, v2

SEQ ID NO: 111 - DNA, 342 bp, Artificial Sequence
  12D3 mature light chain variable region
gacattgtgatgacacagtctccatcctccctgactgtgacagcaggagagaaggtcactatgagctgcaaatccagtcagagtctgttaaacagtggaaatcaaaagaactacttgacctggtaccaacagaaaccagggcagcctcctaaactgttgatctactgggcatccactagggaatctggggtccctgatcgcttcgcaggcagtggctctggaacagatttcactctcaccatcagcagtgtgcaggctgaagacctggcagtttattactgtcagaatgattatgattatccgctcacgttcggtgctgggaccaagctggagctgaaacgg

SEQ ID NO: 112 - DNA, 57 bp, Artificial Sequence
  12D3 heavy chain variable region signal peptide, v1
atgggatggagctatatcatcctctttttggtagcaacagctacaggtgtccactcc

SEQ ID NO: 113 - DNA, 57 bp, Artificial Sequence
  12D3 heavy chain variable region signal peptide, v2
atggaatggatctggatctttctcttcatcctgtcaggaactgcaggtgtccaatcc

SEQ ID NO: 114 - DNA, 60 bp, Artificial Sequence
  12D3 light chain variable region signal peptide
atggaatcacagactcaggtcctcatgtccctgctgttctgggtatctggtacccgtggg

SEQ ID NO: 115 - DNA, 348 bp, Artificial Sequence
  Synthesized
caggttcagctgcagcagtctggggctgagctggtgaagcctggggcctcagtgaagatttcctgcaaagcttctggctacgcattcagtacctactggatgaactgggtgaagcaggcgcctggagagggtcttgagtggattggacagatttatcctggagatggtgatactaactacaacggaaagttcaagggcagggtcacactgactgcagacaagtccaccagcacagcctacatggagctcagcagcctgaggtctgaggacactgcggtctatttctgtgcaagatcagatggttactacgactactggggccaaggcaccactgtcacagtctcctca

SEQ ID NO: 116 - DNA, 348 bp, Artificial Sequence
  Synthesized
caggttcagctgcagcagtctggggctgagctggtgaagcctggggcctcagtgaaggtttcctgcaaagcttctggctacgcattcagtacctactggatgaactgggtgaggcaggcgcctggacagggtcttgagtggattggacagatttatcctggagatggtgatactaactacaacggaaagttcaagggcagggtcacaattactgcagacaagtccaccagcacagcctacatggagctcagcagcctgaggtctgaggacactgcggtctatttctgtgcaagatcagatggttactacgactactggggccaaggcaccactgtcacagtctcctca

SEQ ID NO: 117 - DNA, 348 bp, Artificial Sequence
  Synthesized
gaggttcagctgcagcagtctggggctgagctggtgaagcctggggcctcagtgaaggtttcctgcaaagcttctggctacgcattcagtacctactggatgaactgggtgaggcaggcgcctggacagggtcttgagtggattggacagatttatcctggagatggtgatactaactacaacggaaagttcaagggcagggtcacaattactgcagacaagtccaccagcacagcctacatggagctcagcagcctgaggtctgaggacactgcggtctatttctgtgcaagatcagatggttactacgactactggggccaaggcaccactgtcacagtctcctca

SEQ ID NO: 118 - DNA, 336 bp, Artificial Sequence
  Synthesized
aacattgtgctgacccaatctccagcttctttggctgtgtctctaggggagagggccaccatatcctgcagagccagtgaaagtgttgatagttatggcactagttttatgcactggtaccagcagaaaccaggacagccacccaaactcctcatctgtcttgcatccagcctagaatctggggtccctgacaggttcagtggcagtgggtctaggacagacttcaccctcaccattagtagtctgcaggctgaggatgttgcaacctattactgtcagcaaaataatgaggatcctcctacgttcggtcaggggaccaagctggagattaaacgt

SEQ ID NO: 119 - DNA, 336 bp, Artificial Sequence
  Synthesized
aacattgtgctgacccaatctccagcttctttggctgtgtctctaggggagagggccaccatatcctgcagagccagtgaaagtgttgatagttatggcactagttttatgcactggtaccagcagaaaccaggacagccacccaaactcctcatctctcttgcatccagcctagaatctggggtccctgacaggttcagtggcagtgggtctgggacagacttcaccctcaccattagtagtctgcaggctgaggatgttgcaacctattactgtcagcaaaataatgaggatcctcctacgttcggtcaggggaccaagctggagattaaacgt

SEQ ID NO: 120 - DNA, 336 bp, Artificial Sequence
  Synthesized
aacattgtgctgacccaatctccagcttctttggctgtgtctctaggggagagggccaccatatcctgcagagccagtgaaagtgttgatagttatggcactagttttatgcactggtaccagcagaaaccaggacagccacccaaactcctcatctgtcttgcatccagcctagaatctggggtccctgccaggttcagtggcagtgggtctaggacagacttcaccctcaccattgatcctgtggaggctgaggatgctgcaacctattactgtcagcaaaataatgaggatcctcctacgttcggtgctgggaccaagctggagatcaaacgt

SEQ ID NO: 121 - DNA, 336 bp, Artificial Sequence
  Synthesized
aacattgtgctgacccaatctccagcttctttggctgtgtctctaggggagagggccaccatatcctgcagagccagtgaaagtgttgatagttatggcactagttttatgcactggtaccagcagaaaccaggacagccacccaaactcctcatcagtcttgcatccagcctagaatctggggtccctgccaggttcagtggcagtgggtctgggacagacttcaccctcaccatttctcctgtggaggctgaggatgctgcaacctattactgtcagcaaaataatgaggatcctcctacgttcggtgctgggaccaagctggagatcaaacgt

SEQ ID NO: 122 - DNA, 336 bp, Artificial Sequence
  Synthesized
gacattgtgctgacccaatctccagcttctttggctgtgtctctaggggagagggccaccatatcctgcagagccagtgaaagtgttgatagttatggcactagttttatgcactggtaccagcagaaaccaggacagccacccaaactcctcatcagtcttgcatccagcctagaatctggggtccctgccaggttcagtggcagtgggtctgggacagacttcaccctcaccatttctcctctgcaggctgaggatgttgcaacctattactgtcagcaaaataatgaggatcctcctacgttcggtcaggggaccaagctggagatcaaacgt

SEQ ID NO: 123 - DNA, 336 bp, Artificial Sequence
  Synthesized
gacattgtgctgacccaatctccagcttctttggctgtgtctctaggggagagggccaccatatcctgcagagccagtgaaagtgttgatagttatggcactagttttatgcactggtaccagcagaaaccaggacagccacccaaactcctcatcagtcttgcatccagcctagaatctggggtccctgccaggttcagtggcagtgggtctgggacagacttcaccctcaccatttctcctgtggaggctgaggatgctgcaacctattactgtcagcaaaataatgaggatcctcctacgttcggtgctgggaccaagctggagatcaaacgt

SEQ ID NO: 124 - protein, 823 aa, Artificial Sequence
  G domain of human LAMA4 with LG3 deleted
SKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGRVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLIPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCP

SEQ ID NO: 125 - protein, 786 aa, Artificial Sequence
  G domain of human LAMA4 with LG1 deleted
ARDKLAFTQSRAASYFFDGSGYAVVRDITRRGKFGQVTRFDIEVRTPADNGLILLMVNGSMFFRLEMRNGYLHVFYDFGFSSGRVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCP

SEQ ID NO: 126 - protein, 807 aa, Artificial Sequence
  G domain of human LAMA4 with LG2 deleted
SKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCP

SEQ ID NO: 127 - DNA, 2976 bp, Artificial Sequence
  G domain of human LAMA4
agcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccgtgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactggagcagacagaaaccctgggagttggttatggatgcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtcca

SEQ ID NO: 128 - DNA, 618 bp, Artificial Sequence
  LG1 module of G domain of human LAMA4
agcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgt

SEQ ID NO: 129 - DNA, 540 bp, Artificial Sequence
  LG2 module of G domain of human LAMA4
gccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccgtgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactggagcagacagaaaccctgggagttggttatggatgc

SEQ ID NO: 130 - DNA, 507 bp, Artificial Sequence
  LG3 module of G domain of human LAMA4
tctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgt

SEQ ID NO: 131 - DNA, 1803 bp, Artificial Sequence
  LG1-3 modules of G domain of human LAMA4
agcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccgtgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactggagcagacagaaaccctgggagttggttatggatgcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaaggga

SEQ ID NO: 132 - DNA, 513 bp, Artificial Sequence
  LG4 module of G domain of human LAMA4
tatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgc

SEQ ID NO: 133 - DNA, 522 bp, Artificial Sequence
  LG5 module of G domain of human LAMA4
acaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgt

SEQ ID NO: 134 - DNA, 1107 bp, Artificial Sequence
  LG4-5 modules of G domain of human LAMA4
agaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtcca

SEQ ID NO: 135 - DNA, 2469 bp, Artificial Sequence
  G domain of human LAMA4 with LG3 deleted
agcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccgtgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactggagcagacagaaaccctgggagttggttatggatgcccagaagactcacttatacccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtcca

SEQ ID NO: 136 - DNA, 2358 bp, Artificial Sequence
  G domain of human LAMA4 with LG1 deleted
gcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcagtggccgtgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactggagcagacagaaaccctgggagttggttatggatgcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtcca

SEQ ID NO: 137 - DNA, 2421 bp, Artificial Sequence
  G domain of human LAMA4 with LG2 deleted
agcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagtaacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtcca

SEQ ID NO: 138 - protein, 330 aa, Artificial Sequence
  Exemplary human IgG1 constant region of IgG1 G1m3 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 107 aa, Artificial Sequence [sequence omitted]

SEQ ID NO: 140 - protein, 329 aa, Artificial Sequence
  Exemplary human IgG1 constant region without a C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 119 aa, Artificial Sequence
  1C1 mature heavy chain variable region, version 2
EVQLVESGGGLVKPGGSLKLSCAASGFTFSSYAMSWVRQTPEKRLEWVATISDGISYTYYPDNVKGRFTISRDNAKNNLYLLMSHLKSEDTAMYYCAREGVVALDFDVWGTGTTVTVSS

SEQ ID NO: 142 - protein, 28 aa, Artificial Sequence
  1C1 heavy chain variable region signal peptide, version 2
MSTEHGPLTMNFGLSLIFLVLVLKGVQC

SEQ ID NO: 143 - protein, 5 aa, Artificial Sequence
  1C1 CDR-H1, version 2
SYAMS

SEQ ID NO: 144 - protein, 17 aa, Artificial Sequence
  1C1 CDR-H2, version 2
TISDGISYTYYPDNVKG

SEQ ID NO: 145 - protein, 10 aa, Artificial Sequence
  1C1 CDR-H3, version 2
EGVVALDFDV

SEQ ID NO: 146 - DNA, 357 bp, Artificial Sequence
  1C1 mature heavy chain variable region, version 2
gaagtgcagctggtggagtctgggggaggcttagtgaagcctggagggtccctgaaactctcctgtgcagcctctggattcactttcagtagctatgccatgtcttgggttcgccagactccggaaaagaggctggagtgggtcgcaaccattagtgatggtattagttacacctactatccagacaatgtaaagggccgattcaccatctccagagacaatgccaagaacaacctgtacctgctaatgagccatctgaagtctgaggacacagccatgtattactgtgcaagagagggggtagtagccctggacttcgatgtctggggcacagggaccacggtcaccgtctcctca

SEQ ID NO: 147 - DNA, 84 bp, Artificial Sequence
  1C1 heavy chain variable region signal peptide, version 2
atgagcactgaacacggacccctcaccatgaacttcgggctcagcttgattttccttgtccttgttttaaaaggtgtccagtgt

SEQ ID NO: 148 - DNA, 990 bp, Artificial Sequence
  Exemplary human IgG1 constant region of IgG1 G1m3 allotype
gcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 149 - DNA, 321 bp, Artificial Sequence
  Exemplary human kappa light chain constant region with a N-terminal arginine
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 150 - protein, 330 aa, Artificial Sequence
  Exemplary human IgG1 constant region of IgG1 G1m3 allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - DNA, 318 bp, Artificial Sequence
  Exemplary human kappa light chain constant region without a N-terminal arginine
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2017/0158755 A1
(43) Pub. Date: Jun. 8, 2017

(54) ANTI-LAMININ4 ANTIBODIES SPECIFIC FOR LG1-3

(71) Applicant: PROTHENA BIOSCIENCES LIMITED, Dublin (IE)

(72) Inventors: Kenneth Flanagan, Alameda, CA (US); Stephen Jed Tam, Belmont, CA (US); Yue Liu, Foster City, CA (US); Theodore Yednock, Forest Knolls, CA (US); Robin Barbour, Walnut Creek, CA (US)

(73) Assignee: Prothena Biosciences Limited, Dublin (IE)

(21) Appl. No.: 15/125,569

(22) PCT Filed: Mar. 12, 2015
(86) PCT No.: PCT/IB2015/051789

§ 371 Date: Sep. 12, 2016

Related U.S. Application Data
(60) Provisional application No. 61/952,129, filed on Mar. 12, 2014.
(60) Provisional application No. 62/023,753, filed on Jul. 11, 2014.
(60) Provisional application No. 62/068,286, filed on Oct. 24, 2014.
(60) Provisional application No. 62/086,600, filed on Dec. 2, 2014.

(51) Int. Cl. C07K 16/18

(52) U.S. Cl. CPC C07K 16/18; C07K 2317/76; C07K 2317/56; C07K 2317/565; C07K 2317/24; C07K 2317/71; C07K 2317/14; A61K 2039/505

(57) ABSTRACT

The invention provides antibodies that specifically bind to the LG1-3 modules of the G domain of laminin α4. The antibodies have the capacity to inhibit binding of laminin α4 to MCAM. The antibodies can be used for inhibiting undesired immune responses, treatment of cancer, or treatment of obesity or obesity-related diseases, among other applications.

### CROSS REFERENCE TO RELATED APPLICATIONS

This application claims the benefit of U.S. Application No. 61/952,129, filed Mar. 12, 2014, U.S. Application No. 62/023,753, filed Jul. 11, 2014, U.S. Application No. 62/068,286, filed Oct. 24, 2014, and U.S. Application No. 62/086,600, filed Dec. 2, 2014, each of which is incorporated herein by reference in its entirety for all purposes.

### REFERENCE TO A SEQUENCE LISTING

The Sequence Listing written in file 459013SEQLIST.txt is 199 kilobytes, was created on Mar. 5, 2015, and is hereby incorporated by reference.

### BACKGROUND

A subset of CD4+ T cells, termed TH17 cells (T helper 17 cells), has been implicated in the pathogenesis of a number of undesired immune responses and autoimmune diseases, particularly neuroinflammatory conditions involving CNS infiltration of T cells, such as multiple sclerosis in humans and experimental autoimmune encephalomyelitis (EAE) in mice. See, e.g., Cua et al., Nature 421: 744-748 (2003); Ivonov et al., Cell 126: 1121-1133 (2006). TH17 cells have been reported to secrete a number of select cytokines including IL-17 and IL-22 and to undergo specific recruitment and infiltration of tissue.

MCAM has been reported to be expressed on TH17 cells and to bind to the ligand laminin α4 (WO2012170071). Antibodies to MCAM have been reported to inhibit EAE disease progression. See Flanagan et al., PLoS One 7(7):e40443 (2012)

### SUMMARY OF THE CLAIMED INVENTION

The invention provides antibodies that specifically binds to an epitope within the LG1-3 modules of the G domain of laminin α4 and inhibits binding of laminin α4 to MCAM. Some antibodies bind to an epitope within LG1. Some antibodies bind to an epitope within LG2. Some antibodies bind to an epitope within LG3. Some antibodies binds to an epitope to which both LG1 and LG2 contribute residues or an epitope to which both LG2 and LG3 contribute residues, or an epitope to which both LG1 and LG3 contribute residues or an epitope to which all of LG1, LG2, and LG3 contribute residues. Some antibodies inhibit binding of laminin α4 to an integrin, such as α6β1.

Some antibodies compete with antibody 19C12 characterized by a mature heavy chain variable region of SEQ ID NO:15 and mature light chain variable region of SEQ ID NO:16, or antibody 1C1 characterized by a mature heavy chain variable region of SEQ ID NO:25 and mature light chain variable region of SEQ ID NO:26, or antibody 5Al2 characterized by a mature heavy chain variable region of SEQ ID NO:35 or 36 and mature light chain variable region of SEQ ID NO:37, or antibody 5B5 characterized by a mature heavy chain variable region of SEQ ID NO:50 and mature light chain variable region of SEQ ID NO:51, or antibody 12D3 characterized by a mature heavy chain variable region of SEQ ID NO:60 or 61 and mature light chain variable region of SEQ ID NO:62. Some antibodies compete with antibody 19C12 characterized by a mature heavy chain variable region of SEQ ID NO:15 and mature light chain variable region of SEQ ID NO:16, or antibody 1C1 characterized by a mature heavy chain variable region of SEQ ID NO:25 or 141 and mature light chain variable region of SEQ ID NO:26, or antibody 5Al2 characterized by a mature heavy chain variable region of SEQ ID NO:35 and mature light chain variable region of SEQ ID NO:37, or antibody 5B5 characterized by a mature heavy chain variable region of SEQ ID NO:50 and mature light chain variable region of SEQ ID NO:51, or antibody 12D3 characterized by a mature heavy chain variable region of SEQ ID NO:60 or 61 and mature light chain variable region of SEQ ID NO:62. Some antibodies bind to the same epitope on laminin α4 as 19C12, 1C1, 5Al2, 5B5, or 12D3. Some antibodies comprise three light chain CDRs and three heavy chain CDRs, wherein each CDR has at least 90% sequence identity to a corresponding CDR from the heavy and light chain variable regions of 19C12 (SEQ ID NOS:15 and 16, respectively), 1C1 (SEQ ID NOS:25 and 26, respectively), 5Al2 (SEQ ID NOS:35/36 and 37, respectively), 5B5 (SEQ ID NOS:50 and 51, respectively), or 12D3 (SEQ ID NOS:60/61 and 62, respectively). Some antibodies comprise three light chain CDRs and three heavy chain CDRs, wherein each CDR has at least 90% sequence identity to a corresponding CDR from the heavy and light chain variable regions of 19C12 (SEQ ID NOS:15 and 16, respectively), 1C1 (SEQ ID NOS:25/141 and 26, respectively), 5Al2 (SEQ ID NOS:35 and 37, respectively), 5B5 (SEQ ID NOS:50 and 51, respectively), or 12D3 (SEQ ID NOS:60/61 and 62, respectively). Some antibodies comprise three heavy chain CDRs and three light chain CDRs of 19C12, 1C1, 5Al2, 5B5, or 12D3.

Any of the above antibodies can be a monoclonal antibody. Any can be a chimeric, humanized, veneered, or human. Any can have human IgG1 kappa isotype.

The invention further provides a humanized or chimeric 19C12 antibody that specifically binds to laminin α4, wherein 19C12 is a mouse antibody characterized by a mature heavy chain variable region of SEQ ID NO:15 and a mature light chain variable region of SEQ ID NO:16. Optionally, the antibodies comprise a humanized heavy chain comprising three CDRs of the 19C12 heavy chain variable region (SEQ ID NO:15) and a humanized light chain comprising three CDRs of the 19C12 light chain variable region (SEQ ID NO:16). Optionally, any differences in CDRs of the mature heavy chain variable region and mature light chain variable region from SEQ ID NOS:15 and 16, respectively reside in positions H60-H65.

Optionally the antibody comprises a humanized mature heavy chain variable region having an amino acid sequence at least 90% identical to SEQ ID NO:81 or SEQ ID NO:82 and a humanized mature light chain variable region having an amino acid sequence at least 90% identical to SEQ ID NO:85 or SEQ ID NO:88. Optionally, the antibody comprises three CDRs of the 19C12 heavy chain variable region (SEQ ID NO:15) and three CDRs of the 19C12 light chain variable region (SEQ ID NO:16). Optionally, at least one of positions L9, L22, and L85 is occupied by A, S, and T, respectively, and at least one of positions H11, H12, H16, H27, H28, H48, H91, and H108 is occupied by L, V, A, Y, A, I, F, and T, respectively. Optionally, positions L9, L22, and L85 are occupied by A, S, and T, respectively, and positions H11, H12, H16, H27, H28, H48, H91, and H108 are occupied by L, V, A, Y, A, I, F, and T, respectively. Optionally, at least one of positions L1, L49, L68, L76, L77, L78, L79, and L100 is occupied by N, C, R, D, P, V, E, and A, respectively. Optionally, at least one of positions H1, H20, H38, H43, and H69 is occupied by E, I, K, E, and L, respectively. Optionally, positions L1, L49, and L68 are occupied by N, C, and R, respectively. Optionally, position L1 is occupied by N. Optionally, positions L1, L49, L68, L76, L77, L78, L79, and L100 are occupied by N, C, R, D, P, V, E, and A, respectively. Optionally positions L1, L77, L78, L79, and L100 are occupied by N, P, V, E, and A, respectively. Optionally position L77 is occupied by P. Optionally positions L77, L78, L79, and L100 are occupied by P, V, E, and A, respectively. Optionally positions H20, H38, H43, and H69 are occupied by I, K, E, and L, respectively. Optionally position H1 is occupied by E.

Some humanized antibodies comprise a mature heavy chain variable region having an amino acid sequence at least 95% identical to SEQ ID NO:81 or SEQ ID NO:82 and a mature light chain variable region having an amino acid sequence at least 95% identical to SEQ ID NO:85 or SEQ ID NO:88. Optionally, the mature heavy chain variable region has an amino acid sequence of SEQ ID NO:81 and the mature light chain variable region has an amino acid sequence of SEQ ID NO:85. Optionally, the mature heavy chain variable region has an amino acid sequence of SEQ ID NO:81 and the mature light chain variable region has an amino acid sequence of SEQ ID NO:86. Optionally, the mature heavy chain variable region has an amino acid sequence of SEQ ID NO:81 and the mature light chain variable region has an amino acid sequence of SEQ ID NO:88. Optionally, the mature heavy chain variable region has an amino acid sequence of SEQ ID NO:82 and the mature light chain variable region has an amino acid sequence of SEQ ID NO:88.

Any of the above antibodies can be an intact antibody, a single-chain antibody, Fab, or Fab′2 fragment. In any of the above antibodies, the mature light chain variable region can be fused to a light chain constant region and the mature heavy chain variable region can be fused to a heavy chain constant region. Optionally, the heavy chain constant region is a mutant form of a natural human heavy chain constant region which has reduced binding to a Fcγ receptor relative to the natural human heavy chain constant region. Optionally, the heavy chain constant region is of IgG1 isotype. Optionally, the mature heavy chain variable region is fused to a heavy chain constant region having the sequence of SEQ ID NO:89 and/or the mature light chain variable region is fused to a light chain constant region having the sequence of SEQ ID NO:90. Optionally, the mature heavy chain variable region is fused to a heavy chain constant region having the sequence of SEQ ID NO:89, 138, or 150 and/or the mature light chain variable region is fused to a light chain constant region having the sequence of SEQ ID NO:90 or 139.

The invention further provides pharmaceutical compositions comprising any of the above described antibodies and a pharmaceutically acceptable carrier.

The invention further provides nucleic acids encoding the heavy and/or light chain(s) of any of the above described antibodies, such as any of SEQ ID NOS:91-92, 95-96, 99-101, 105-106, 109-111, and 115-123. The invention further provides nucleic acids encoding the heavy and/or light chain(s) of any of the above described antibodies, such as any of SEQ ID NOS:91-92, 95-96, 99, 101, 105-106, 109-111, 115-123, 146, 148, 149, or 151.

The invention further provides a recombinant expression vector comprising a nucleic acid as described above, and a host cell transformed with the recombinant expression vector.

The invention further provides a method of humanizing an antibody, the method comprising: (a) determining the sequences of the heavy and light chain variable regions of a mouse antibody; (b) synthesizing a nucleic acid encoding a humanized heavy chain comprising CDRs of the mouse antibody heavy chain and a nucleic acid encoding a humanized light chain comprising CDRs of the mouse antibody light chain; (c) expressing the nucleic acids in a host cell to produce a humanized antibody; wherein the mouse antibody is 19C12, 1C1, 5Al2, 5B5, or 12D3.

The invention further provides a method of producing a humanized, chimeric, or veneered antibody, the method comprising: (a) culturing cells transformed with nucleic acids encoding the heavy and light chains of the antibody, so that the cells secrete the antibody; and (b) purifying the antibody from cell culture media; wherein the antibody is a humanized, chimeric, or veneered form of 19C12, 1C1, 5Al2, 5B5, or 12D3.

The invention further provides a method of producing a cell line producing a humanized, chimeric, or veneered antibody, the method comprising: (a) introducing a vector encoding heavy and light chains of an antibody and a selectable marker into cells; (b) propagating the cells under conditions to select for cells having increased copy number of the vector; (c) isolating single cells from the selected cells; and(d) banking cells cloned from a single cell selected based on yield of antibody; wherein the antibody is a humanized, chimeric, or veneered form of 19C12, 1C1, 5Al2, 5B5, or 12D3. Optionally the method further comprises propagating the cells under selective conditions and screening for cell lines naturally expressing and secreting at least 100 mg/L/10⁶ cells/24 h.

The invention further provides a method of suppressing an undesired immune response in a patient, the method comprising administering to a patient an effective regime of any of the above described antibodies. Optionally, the undesired immune response is characterized by infiltration of MCAM-expressing cells to a site of inflammation. Optionally the MCAM-expressing cells are TH17 cells. Optionally, the undesired immune response is an autoimmune disease, such as diabetes, Crohn's disease, ulcerative colitis, multiple sclerosis, stiff man syndrome, rheumatoid arthritis, myasthenia gravis, systemic lupus erythematosus, celiac disease, psoriasis, psoriatic arthritis, sarcoidosis, ankylosing spondylitis, Sjogren's syndrome, or uveitis, or graft versus host disease or transplant rejection, or an allergy, allergic response, or allergic disease, such as allergic contact dermatitis or asthma.

The invention further provides a method of treating or effecting prophylaxis of a cancer in a patient having or at risk for the cancer, the method comprising administering to a patient an effective regime of any of the above described antibodies. Optionally, the cancer is melanoma, glioma, glioblastoma, lung cancer, or breast cancer. Optionally the cancer is metastatic.

The invention further provides a method of treating or effecting prophylaxis of obesity or an obesity-related disease in a patient having or at risk for obesity or the obesity-related disease, the method comprising administering to a patient an effective regime of any of the above described antibodies. Optionally, the obesity-related disease is non-alcoholic steatohepatitis (NASH), Prader-Willi syndrome, craniopharyngioma, Bardet-Biedl syndrome, Cohen syndrome, or MOMO syndrome.

The invention further provides a method of inhibiting binding of laminin α4 to MCAM in a biological sample, the method comprising contacting the biological sample with an effective amount of any of the above described antibodies.

The invention further provides a method off inhibiting binding of laminin α4 to integrin α6β1 in a biological sample, the method comprising contacting the biological sample with an effective amount of any of the above described antibodies.

The invention further provides a method of inhibiting cell adhesion in a biological sample, the method comprising contacting the biological sample with an effective amount of any of the above antibodies. Optionally the cell adhesion is mediated by the LG1-3 modules of the G domain of laminin α4. Optionally the biological sample comprises cancer cells.

The invention further provides a method of inhibiting angiogenesis in a patient, the method comprising administering to a patient an effective regime of any of the above antibodies. Optionally the patient has a cancer.

### BRIEF DESCRIPTION OF THE DRAWINGS

FIG. 1 shows the ability of IgG control antibody, 1C1, 5Al2, 5B5, 19C12, and 12D3 to block MCAM-LAMA4 binding as assessed by an ELISA hMCAM-Fc capture blocking assay.

FIG. 2A & B show the ability of IgG control antibody, 1C1, 5Al2, 5B5, 19C12, and 12D3 to block MCAM-LAMA4 binding as assessed by a LAMA4 pDisplay flow cytometric blocking assay.

FIG. 3 shows the ability of IgG control antibody, 1C1, 5Al2, 5B5, 19C12, and 12D3 to block MCAM-LAMA4 binding as assessed by a hMCAM.CHO flow cytometric blocking assay.

FIG. 4A-E show the relative binding and on/off rates ability of the 19C12, 1C1, 5Al2, 5B5, and 12D3 antibodies, respectively.

FIG. 5 shows binding of IgG control antibody, 1C1, 5Al2, 5B5, 19C12, and 12D3 to LAMA4-displaying human 293 cells.

FIG. 6 shows the ability of truncated recombinant variants of the LAMA4 G domain to bind MCAM-Fc protein as assessed by ELISA, with Tau protein used as a control.

FIG. 7A & B show binding as assessed by flow cytometry of 293 cells displaying LAMA 4 variants with LG1-5, LG1-3, and LG4-5 (FIG. 7A) and LAMA 4 variants with LG1-3, G domain with LG1 deleted, G domain with LG2 deleted, and G domain with LG3 deleted (FIG. 7B).

FIG. 8A-E show assessment of binding by flow cytometry of the 5Al2, 19C12, 1C1, 5B5, and 12D3 antibodies, respectively, to LAMA4-displaying 293 cells in the presence of decreasing ratios (5:1, 1:1, and 1:5) of competing blocking antibodies.

FIG. 9 shows the ability of 19C12 and a mouse IgG2b control to block LAMA4-mediated WM-266-4 cell adhesion.

FIG. 10 shows the ability of 19C12 to block LAMA4 binding to integrin-α6β1-expressing 293 cells as demonstrated by flow cytometry analysis.

FIG. 11 shows the ability of chimeric 19C12, H1+ChiL, and H2+ChiL to block the binding of LAMA4 to MCAM-expressing CHO cells as assessed by flow cytometry.

FIG. 12 shows the flow cytometry assessment of the ability of chimeric 19C12, H1+ChiL, and H2+ChiL to bind to 293 cells displaying recombinant variants of the LAMA4 G domain.

FIG. 13 shows the ability of humanized 19C12 variants with amino acid substitutions at position L49 to block the binding of LAMA4 to MCAM-expressing CHO cells as assessed by flow cytometry.

FIG. 14 shows the ability of humanized 19C12 variants with amino acid substitutions at position L49 to bind to LAMA4-displaying 293 cells as assessed by flow cytometry.

FIG. 15 shows the ability of chimeric 19C12, H2L3, H2L4, H2L6, and H3L6 to block the binding of LAMA4 to MCAM-expressing CHO cells as assessed by flow cytometry.

FIG. 16 shows the ability of chimeric 19C12, H2L3, H2L4, H2L6, and H3L6 to bind to LAMA4-displaying 293 cells as assessed by flow cytometry.

FIG. 17A & B show relative binding and on/off rates for chimeric 19C12 and humanized 15F7 variants H2L3, H2L4, H2L6, and H3L6 as assessed by ForteBio, with the anti-His sensor being loaded with His-LAMA4 followed by association and dissociation of the 19C12 antibodies in 17A, and the goat anti-human Fc sensor being loaded with the antibodies followed by association and dissociation of His-LAMA4 in 17B.

FIG. 18A-C show the relative binding and on/off rates ability of chimeric 19C12 and humanized 15F7 variants H2L3, H2L4, H2L6, and H3L6 as assessed by ForteBio, with antibody concentrations of 33.3 nM, 16.7 nM, and 8.33 nM in 18A-C, respectively.

FIG. 19A & B show ratios of the relative levels of pAkt to Akt in human melanoma cells treated with laminin 411 or BSA control and with 19C12, 4B7, r2107, or mIgG2b control. FIG. 19A shows the ratio for each individual sample, and FIG. 19B shows averages and standard errors for each group (n=3).

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO:1 sets forth the amino acid sequence of laminin α4 as provided by UniProt Number Q16363.

SEQ ID NO:2 sets forth the amino acid sequence of laminin α4 as provided by GenBank Accession Number NP001098676.

SEQ ID NO:3 sets forth the amino acid sequence of laminin α4 as provided by GenBank Accession Number NP001098677.

SEQ ID NO:4 sets forth the amino acid sequence of the G domain of laminin α4.

SEQ ID NO:5 sets forth the amino acid sequence of the LG1 module of the G domain of laminin α4.

SEQ ID NO:6 sets forth the amino acid sequence of the LG2 module of the G domain of laminin α4.

SEQ ID NO:7 sets forth the amino acid sequence of the LG3 module of the G domain of laminin α4.

SEQ ID NO:8 sets forth the amino acid sequence of the LG1-3 modules of the G domain of laminin α4.

SEQ ID NO:9 sets forth the amino acid sequence of the LG4 module of the G domain of laminin α4.

SEQ ID NO:10 sets forth the amino acid sequence of the LG5 module of the G domain of laminin α4.

SEQ ID NO:11 sets forth the amino acid sequence of the LG4-5 modules of the G domain of laminin α4.

SEQ ID NO:12 sets forth the amino acid sequence of MCAM as provided by UniProt Number P43121.

SEQ ID NO:13 sets forth the amino acid sequence of integrin α6 as provided by UniProt Number P23229.

SEQ ID NO:14 sets forth the amino acid sequence of integrin β1 as provided by UniProt Number P05556.

SEQ ID NO:15 sets forth the amino acid sequence of mouse 19C12 mature heavy chain variable region.

SEQ ID NO:16 sets forth the amino acid sequence of mouse 19C12 mature light chain variable region.

SEQ ID NO:17 sets forth the amino acid sequence of the 19C12 heavy chain variable region signal peptide.

SEQ ID NO:18 sets forth the amino acid sequence of the 19C12 light chain variable region signal peptide.

SEQ ID NO:19 sets forth the amino acid sequence of CDR1, as defined by Kabat, of the mouse 19C12 heavy chain.

SEQ ID NO:20 sets forth the amino acid sequence of CDR2, as defined by Kabat, of the mouse 19C12 heavy chain.

SEQ ID NO:21 sets forth the amino acid sequence of CDR3, as defined by Kabat, of the mouse 19C12 heavy chain.

SEQ ID NO:22 sets forth the amino acid sequence of CDR1, as defined by Kabat, of the mouse 19C12 light chain.

SEQ ID NO:23 sets forth the amino acid sequence of CDR2, as defined by Kabat, of the mouse 19C12 light chain.

SEQ ID NO:24 sets forth the amino acid sequence of CDR3, as defined by Kabat, of the mouse 19C12 light chain.

SEQ ID NO:25 sets forth the amino acid sequence of mouse 1C1 mature heavy chain variable region, version 1.

SEQ ID NO:26 sets forth the amino acid sequence of mouse 1C1 mature light chain variable region.

SEQ ID NO:27 sets forth the amino acid sequence of the 1C1 heavy chain variable region signal peptide, version 1.

SEQ ID NO:28 sets forth the amino acid sequence of the 1C1 light chain variable region signal peptide.

SEQ ID NO:29 sets forth the amino acid sequence of CDR1, as defined by Kabat, of the mouse 1C1 heavy chain, version 1.

SEQ ID NO:30 sets forth the amino acid sequence of CDR2, as defined by Kabat, of the mouse 1C1 heavy chain, version 1.

SEQ ID NO:31 sets forth the amino acid sequence of CDR3, as defined by Kabat, of the mouse 1C1 heavy chain, version 1.

SEQ ID NO:32 sets forth the amino acid sequence of CDR1, as defined by Kabat, of the mouse 1C1 light chain.

SEQ ID NO:33 sets forth the amino acid sequence of CDR2, as defined by Kabat, of the mouse 1C1 light chain.

SEQ ID NO:34 sets forth the amino acid sequence of CDR3, as defined by Kabat, of the mouse 1C1 light chain.

SEQ ID NO:35 sets forth the amino acid sequence of mouse 5Al2 mature heavy chain variable region, version 1.

SEQ ID NO:36 sets forth the amino acid sequence of mouse 5Al2 mature heavy chain variable region, version 2.

SEQ ID NO:37 sets forth the amino acid sequence of mouse 5Al2 mature light chain variable region.

SEQ ID NO:38 sets forth the amino acid sequence of the 5Al2 heavy chain variable region signal peptide, version 1.

SEQ ID NO:39 sets forth the amino acid sequence of the 5Al2 heavy chain variable region signal peptide, version 2.

SEQ ID NO:40 sets forth the amino acid sequence of the 5Al2 light chain variable region signal peptide.

SEQ ID NO:41 sets forth the amino acid sequence of CDR1, as defined by Kabat, of the mouse 5Al2 heavy chain, version 1.

SEQ ID NO:42 sets forth the amino acid sequence of CDR2, as defined by Kabat, of the mouse 5Al2 heavy chain, version 1.

SEQ ID NO:43 sets forth the amino acid sequence of CDR3, as defined by Kabat, of the mouse 5Al2 heavy chain, version 1.

SEQ ID NO:44 sets forth the amino acid sequence of CDR1, as defined by Kabat, of the mouse 5Al2 heavy chain, version 2.

SEQ ID NO:45 sets forth the amino acid sequence of CDR2, as defined by Kabat, of the mouse 5Al2 heavy chain, version 2.

SEQ ID NO:46 sets forth the amino acid sequence of CDR3, as defined by Kabat, of the mouse 5Al2 heavy chain, version 2.

SEQ ID NO:47 sets forth the amino acid sequence of CDR1, as defined by Kabat, of the mouse 5Al2 light chain.

SEQ ID NO:48 sets forth the amino acid sequence of CDR2, as defined by Kabat, of the mouse 5Al2 light chain.

SEQ ID NO:49 sets forth the amino acid sequence of CDR3, as defined by Kabat, of the mouse 5Al2 light chain.

SEQ ID NO:50 sets forth the amino acid sequence of mouse 5B5 mature heavy chain variable region.

SEQ ID NO:51 sets forth the amino acid sequence of mouse 5B5 mature light chain variable region.

SEQ ID NO:52 sets forth the amino acid sequence of the 5B5 heavy chain variable region signal peptide.

SEQ ID NO:53 sets forth the amino acid sequence of the 5B5 light chain variable region signal peptide.

SEQ ID NO:54 sets forth the amino acid sequence of CDR1, as defined by Kabat, of the mouse 5B5 heavy chain.

SEQ ID NO:55 sets forth the amino acid sequence of CDR2, as defined by Kabat, of the mouse 5B5 heavy chain.

SEQ ID NO:56 sets forth the amino acid sequence of CDR3, as defined by Kabat, of the mouse 5B5 heavy chain.

SEQ ID NO:57 sets forth the amino acid sequence of CDR1, as defined by Kabat, of the mouse 5B5 light chain.

SEQ ID NO:58 sets forth the amino acid sequence of CDR2, as defined by Kabat, of the mouse 5B5 light chain.

SEQ ID NO:59 sets forth the amino acid sequence of CDR3, as defined by Kabat, of the mouse 5B5 light chain.

SEQ ID NO:60 sets forth the amino acid sequence of mouse 12D3 mature heavy chain variable region, version 1.

SEQ ID NO:61 sets forth the amino acid sequence of mouse 12D3 mature heavy chain variable region, version 2.

SEQ ID NO:62 sets forth the amino acid sequence of mouse 12D3 mature light chain variable region.

SEQ ID NO:63 sets forth the amino acid sequence of the 12D3 heavy chain variable region signal peptide, version 1.

SEQ ID NO:64 sets forth the amino acid sequence of the 12D3 heavy chain variable region signal peptide, version 2.

SEQ ID NO:65 sets forth the amino acid sequence of the 12D3 light chain variable region signal peptide.

SEQ ID NO:66 sets forth the amino acid sequence of CDR1, as defined by Kabat, of the mouse 12D3 heavy chain, version 1.

SEQ ID NO:67 sets forth the amino acid sequence of CDR2, as defined by Kabat, of the mouse 12D3 heavy chain, version 1.

SEQ ID NO:68 sets forth the amino acid sequence of CDR3, as defined by Kabat, of the mouse 12D3 heavy chain, version 1.

SEQ ID NO:69 sets forth the amino acid sequence of CDR1, as defined by Kabat, of the mouse 12D3 heavy chain, version 2.

SEQ ID NO:70 sets forth the amino acid sequence of CDR2, as defined by Kabat, of the mouse 12D3 heavy chain, version 2.

SEQ ID NO:71 sets forth the amino acid sequence of CDR3, as defined by Kabat, of the mouse 12D3 heavy chain, version 2.

SEQ ID NO:72 sets forth the amino acid sequence of CDR1, as defined by Kabat, of the mouse 12D3 light chain.

SEQ ID NO:73 sets forth the amino acid sequence of CDR2, as defined by Kabat, of the mouse 12D3 light chain.

SEQ ID NO:74 sets forth the amino acid sequence of CDR3, as defined by Kabat, of the mouse 12D3 light chain.

SEQ ID NO:75 sets forth the amino acid sequence of a human VH acceptor FR as provided by NCBI Accession Code BAC01530.1.

SEQ ID NO:76 sets forth the amino acid sequence of a human VL acceptor FR as provided by NCBI Accession Code ABA71367.1.

SEQ ID NO:77 sets forth the amino acid sequence of a human VL acceptor FR as provided by NCBI Accession Code ABI74162.1.

SEQ ID NO:78 sets forth the amino acid sequence of humanized 19C12 heavy chain variable region with no backmutations or other mutations.

SEQ ID NO:79 sets forth the amino acid sequence of humanized 19C12 light chain variable region with no backmutations or other mutations.

SEQ ID NO:80 sets forth the amino acid sequence of humanized 19C12 heavy chain variable region version 1 (H1).

SEQ ID NO:81 sets forth the amino acid sequence of humanized 19C12 heavy chain variable region version 2 (H2).

SEQ ID NO:82 sets forth the amino acid sequence of humanized 19C12 heavy chain variable region version 3 (H3).

SEQ ID NO:83 sets forth the amino acid sequence of humanized 19C12 light chain variable region version 1 (L1).

SEQ ID NO:84 sets forth the amino acid sequence of humanized 19C12 light chain variable region version 2 (L2).

SEQ ID NO:85 sets forth the amino acid sequence of humanized 19C12 light chain variable region version 3 (L3).

SEQ ID NO:86 sets forth the amino acid sequence of humanized 19C12 light chain variable region version 4 (L4).

SEQ ID NO:87 sets forth the amino acid sequence of humanized 19C12 light chain variable region version 5 (L5).

SEQ ID NO:88 sets forth the amino acid sequence of humanized 19C12 light chain variable region version 6 (L6).

SEQ ID NO:89 sets forth the amino acid sequence of an exemplary human IgG1 constant region.

SEQ ID NO:90 sets forth the amino acid sequence of an exemplary human kappa light chain constant region without a N-terminal arginine.

SEQ ID NO:91 sets forth the nucleic acid sequence of mouse 19C12 mature heavy chain variable region.

SEQ ID NO:92 sets forth the nucleic acid sequence of mouse 19C12 mature light chain variable region.

SEQ ID NO:93 sets forth the nucleic acid sequence of the 19C12 heavy chain variable region signal peptide.

SEQ ID NO:94 sets forth the nucleic acid sequence of the 19C12 light chain variable region signal peptide.

SEQ ID NO:95 sets forth the nucleic acid sequence of mouse 1C1 mature heavy chain variable region, version 1.

SEQ ID NO:96 sets forth the nucleic acid sequence of mouse 1C1 mature light chain variable region.

SEQ ID NO:97 sets forth the nucleic acid sequence of the 1C1 heavy chain variable region signal peptide, version 1.

SEQ ID NO:98 sets forth the nucleic acid sequence of the 1C1 light chain variable region signal peptide.

SEQ ID NO:99 sets forth the nucleic acid sequence of mouse 5Al2 mature heavy chain variable region, version 1.

SEQ ID NO:100 sets forth the nucleic acid sequence of mouse 5Al2 mature heavy chain variable region, version 2.

SEQ ID NO:101 sets forth the nucleic acid sequence of mouse 5Al2 mature light chain variable region.

SEQ ID NO:102 sets forth the nucleic acid sequence of the 5Al2 heavy chain variable region signal peptide, version 1.

SEQ ID NO:103 sets forth the nucleic acid sequence of the 5Al2 heavy chain variable region signal peptide, version 2.

SEQ ID NO:104 sets forth the nucleic acid sequence of the 5Al2 light chain variable region signal peptide.

SEQ ID NO:105 sets forth the nucleic acid sequence of mouse 5B5 mature heavy chain variable region.

SEQ ID NO:106 sets forth the nucleic acid sequence of mouse 5B5 mature light chain variable region.

SEQ ID NO:107 sets forth the nucleic acid sequence of the 5B5 heavy chain variable region signal peptide.

SEQ ID NO:108 sets forth the nucleic acid sequence of the 5B5 light chain variable region signal peptide.

SEQ ID NO:109 sets forth the nucleic acid sequence of mouse 12D3 mature heavy chain variable region, version 1.

SEQ ID NO:110 sets forth the nucleic acid sequence of mouse 12D3 mature heavy chain variable region, version 2.

SEQ ID NO:111 sets forth the nucleic acid sequence of mouse 12D3 mature light chain variable region.

SEQ ID NO:112 sets forth the nucleic acid sequence of the 12D3 heavy chain variable region signal peptide, version 1.

SEQ ID NO:113 sets forth the nucleic acid sequence of the 12D3 heavy chain variable region signal peptide, version 2.

SEQ ID NO:114 sets forth the nucleic acid sequence of the 12D3 light chain variable region signal peptide.

SEQ ID NO:115 sets forth the nucleic acid sequence of humanized 19C12 heavy chain variable region version 1 (H1).

SEQ ID NO:116 sets forth the nucleic acid sequence of humanized 19C12 heavy chain variable region version 2 (H2).

SEQ ID NO:117 sets forth the nucleic acid sequence of humanized 19C12 heavy chain variable region version 3 (H3).

SEQ ID NO:118 sets forth the nucleic acid sequence of humanized 19C12 light chain variable region version 1 (L1).

SEQ ID NO:119 sets forth the nucleic acid sequence of humanized 19C12 light chain variable region version 2 (L2).

SEQ ID NO:120 sets forth the nucleic acid sequence of humanized 19C12 light chain variable region version 3 (L3).

SEQ ID NO:121 sets forth the nucleic acid sequence of humanized 19C12 light chain variable region version 4 (L4).

SEQ ID NO:122 sets forth the nucleic acid sequence of humanized 19C12 light chain variable region version 5 (L5).

SEQ ID NO:123 sets forth the nucleic acid sequence of humanized 19C12 light chain variable region version 6 (L6).

SEQ ID NO:124 sets forth the amino acid sequence of of LGde3, a mutant of the G domain of laminin α4 with LG3 deleted.

SEQ ID NO:125 sets forth the amino acid sequence of LGde1, a mutant of the G domain of laminin α4 with LG1 deleted.

SEQ ID NO:126 sets forth the amino acid sequence of LGde2, a mutant of the G domain of laminin α4 with LG2 deleted.

SEQ ID NO:127 sets forth the nucleic acid sequence of the G domain of laminin α4.

SEQ ID NO:128 sets forth the nucleic acid sequence of the LG1 module of the G domain of laminin α4.

SEQ ID NO:129 sets forth the nucleic acid sequence of the LG2 module of the G domain of laminin α4.

SEQ ID NO:130 sets forth the nucleic acid sequence of the LG3 module of the G domain of laminin α4.

SEQ ID NO:131 sets forth the nucleic acid sequence of the LG1-3 modules of the G domain of laminin α4.

SEQ ID NO:132 sets forth the nucleic acid sequence of the LG4 module of the G domain of laminin α4.

SEQ ID NO:133 sets forth the nucleic acid sequence of the LG5 module of the G domain of laminin α4.

SEQ ID NO:134 sets forth the nucleic acid sequence of the LG4-5 modules of the G domain of laminin α4.

SEQ ID NO:135 sets forth the nucleic acid sequence of LGde3, a mutant of the G domain of laminin α4 with LG3 deleted.

SEQ ID NO:136 sets forth the nucleic acid sequence of LGde1, a mutant of the G domain of laminin α4 with LG1 deleted.

SEQ ID NO:137 sets forth the nucleic acid sequence of LGde2, a mutant of the G domain of laminin α4 with LG2 deleted.

SEQ ID NO:138 sets forth the amino acid sequence of an exemplary human IgG1 constant region of the IgG1 G1m3 allotype.

SEQ ID NO:139 sets forth the amino acid sequence of an exemplary human kappa light chain constant region with a N-terminal arginine.

SEQ ID NO:140 sets forth the amino acid sequence of an exemplary human IgG1 constant region without a C-terminal lysine.

SEQ ID NO:141 sets forth the amino acid sequence of mouse 1C1 mature heavy chain variable region, version 2.

SEQ ID NO:142 sets forth the amino acid sequence of the 1C1 heavy chain variable region signal peptide, version 2.

SEQ ID NO:143 sets forth the amino acid sequence of CDR1, as defined by Kabat, of the mouse 1C1 heavy chain, version 2.

SEQ ID NO:144 sets forth the amino acid sequence of CDR2, as defined by Kabat, of the mouse 1C1 heavy chain, version 2.

SEQ ID NO:145 sets forth the amino acid sequence of CDR3, as defined by Kabat, of the mouse 1C1 heavy chain, version 2.

SEQ ID NO:146 sets forth the nucleic acid sequence of mouse 1C1 mature heavy chain variable region, version 2.

SEQ ID NO:147 sets forth the nucleic acid sequence of the 1C1 heavy chain variable region signal peptide, version 2.

SEQ ID NO:148 sets forth the nucleic acid sequence of an exemplary human IgG1 constant region of the IgG1 G1m3 allotype.

SEQ ID NO:149 sets forth the nucleic acid sequence of an exemplary human kappa light chain constant region with a N-terminal arginine.

SEQ ID NO:150 sets forth the amino acid sequence of an exemplary human IgG1 constant region of the IgG1 G1m3 allotype.

SEQ ID NO:151 sets forth the nucleic acid sequence of an exemplary human kappa light chain constant region without a N-terminal arginine.

### DEFINITIONS

Monoclonal antibodies or other biological entities are typically provided in isolated form. This means that an antibody or other biologically entity is typically at least 50% w/w pure of interfering proteins and other contaminants arising from its production or purification but does not exclude the possibility that the monoclonal antibody is combined with an excess of pharmaceutically acceptable carrier(s) or other vehicle intended to facilitate its use. Sometimes monoclonal antibodies are at least 60%, 70%, 80%, 90%, 95% or 99% w/w pure of interfering proteins and contaminants from production or purification. Often an isolated monoclonal antibody or other biological entity is the predominant macromolecular species remaining after its purification.

Specific binding of an antibody to its target antigen means an affinity of at least 10⁶, 10⁷, 10⁸, 10⁹, or 10¹⁰ M. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. See generally, Fundamental Immunology (Paul, W., ed., 2nd ed. Raven Press, N.Y., 1989), Ch. 7 (incorporated by reference in its entirety for all purposes).

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chains or between different light chains are assigned the same number.

The term “antibody” includes intact antibodies and binding fragments thereof. Typically, fragments compete with the intact antibody from which they were derived for specific binding to the target including separate heavy chains, light chains Fab, Fab′, F(ab′)₂, F(ab)c, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)). In some bispecific antibodies, the two different heavy/light chain pairs include a humanized 19C12 heavy chain/light chain pair and a heavy chain/light chain pair specific for a different epitope on laminin α4 than that bound by 19C12.

In some bispecific antibodies, one heavy chain light chain pair is a humanized 19C12 antibody as further disclosed below and the heavy light chain pair is from an antibody that binds to a receptor expressed on the blood brain barrier, such as an insulin receptor, an insulin-like growth factor (IGF) receptor, a leptin receptor, or a lipoprotein receptor, or a transferrin receptor (Friden et al., PNAS 88:4771-4775, 1991; Friden et al., Science 259:373-377, 1993). Such a bispecific antibody can be transferred cross the blood brain barrier by receptor-mediated transcytosis. Brain uptake of the bispecific antibody can be further enhanced by engineering the bi-specific antibody to reduce its affinity to the blood brain barrier receptor. Reduced affinity for the receptor resulted in a broader distributioin in the brain (see, e.g., Atwal. et al., Sci. Trans. Med. 3, 84rα43, 2011; Yu et al., Sci. Trans. Med. 3, 84rα44, 2011).

Exemplary bispecific antibodies can also be (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (5) a so-called Scorpion molecule, comprising, e.g., two scFvs fused to both termini of a human Fc-region. Examples of platforms useful for preparing bispecific antibodies include BiTE (Micromet), DART (MacroGenics), Fcab and Mab2 (F-star), Fc-engineered IgG1 (Xencor) or DuoBody (based on Fab arm exchange, Genmab).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% as measured in a competitive binding assay. Some test antibodies inhibit binding of the references antibody by at least 75%, 90% or 99%. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “pharmaceutically acceptable” means that the carrier, diluent, excipient, or auxiliary is compatible with the other ingredients of the formulation and not substantially deleterious to the recipient thereof.

The term “patient” includes human and other mammalian subjects that receive either prophylactic or therapeutic treatment.

An individual is at increased risk of a disease if the subject has at least one known risk-factor (e.g., genetic, biochemical, family history, situational exposure) placing individuals with that risk factor at a statistically significant greater risk of developing the disease than individuals without the risk factor.

The term “biological sample” refers to a sample of biological material within or obtainable from a biological source, for example a human or mammalian subject. Such samples can be organs, organelles, tissues, sections of tissues, bodily fluids, peripheral blood, blood plasma, blood serum, cells, molecules such as proteins and peptides, and any parts or combinations derived therefrom. The term biological sample can also encompass any material derived by processing the sample. Derived material can include cells or their progeny. Processing of the biological sample may involve one or more of filtration, distillation, extraction, concentration, fixation, inactivation of interfering components, and the like.

The term “symptom” refers to a subjective evidence of a disease, such as altered gait, as perceived by the subject. A “sign” refers to objective evidence of a disease as observed by a physician.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” or “including” one or more recited elements may include other elements not specifically recited. For example, a composition that “comprises” or “includes” an antibody may contain the antibody alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value.

Statistical significance means p.0.05.

The singular forms of the articles “a,” “an,” and “the” include plural references unless the context clearly dictates otherwise. For example, the term “a compound” or “at least one compound” can include a plurality of compounds, including mixtures thereof.

### DETAILED DESCRIPTION

### I. General

The invention provides antibodies that specifically bind to the LG1-3 modules of the G domain of laminin α4. The antibodies have the capacity to inhibit binding of laminin α4 to MCAM and optionally to integrin α6β1. The antibodies can be used for inhibiting undesired immune responses, treatment of cancer, or treatment of obesity or obesity-related diseases, among other applications.

### II. Target Molecules

Laminins are a family of extracellular matrix glycoproteins and are the major non-collagenous constitutent of basement membranes. They have been reported to be involved in biological processes including cell adhesion, differentiation, migration, signaling, neurite outgrowth, and metastasis, among other processes. Laminins are heterotrimeric proteins of three chains: an alpha chain, a beta chain, and a gamma chain. The three chains form a cruciform structure consisting of three short arms, each formed by a different chain, and a long arm composed of all three chains. In mammals, five different alpha chains, three different beta chains, and three different gamma chains have been identified that can assemble into fifteen different heterotrimeric combinations.

The laminin alpha chains have a large C-terminal globular domain (G domain) that has five tandem homologous laminin G-like modules (LG1-5) of about 200 amino acids. For example, the G domain of laminin α4 is defined by UniProt sequence Q16363 as amino acid positions 833-1820 (SEQ ID NO:4), and the five LG modules of laminin α4 are defined by UniProt sequence Q16363 as follows: LG1 (SEQ ID NO:5) includes amino acid positions 833-1035, LG2 (SEQ ID NO:6) includes amino acid positions 1047-1227, LG3 (SEQ ID NO:7) includes amino acid positions 1234-1402, LG4 (SEQ ID NO:9) includes amino acid positions 1469-1640, and LG5 (SEQ ID NO:10) includes amino acid positions 1647-1820. In some cases, the G domain can be SEQ ID NO:4; in other cases it can include amino acid positions 833-1820 of UniProt sequence Q16363. In some cases, the LG1 module can be SEQ ID NO:5; in other cases it can include amino acid positions 833-1035 of UniProt sequence Q16363. In some cases, the LG2 module can be SEQ ID NO:6; in other cases it can include amino acid positions 1047-1227 of UniProt sequence Q16363. In some cases, the LG3 module can be SEQ ID NO:7; in other cases it can include amino acid positions 1234-1402 of UniProt sequence Q16363. In some cases, the LG4 module can be SEQ ID NO:9; in other cases it can include amino acid positions 1469-1640 of UniProt sequence Q16363. In some cases, the LG5 module can be SEQ ID NO:10; in other cases it can include amino acid positions 1647-1820 of UniProt sequence Q16363. The LG1-3 modules (SEQ ID NO:8) are connected to the LG4-5 modules (SEQ ID NO:11) by a linker domain. The laminin α4 chain (also known as LAMA4, laminin subunit α4, laminin-14 subunit alpha, laminin-8 subunit alpha, and laminin-9 subunit alpha) is 200 kDa and is the shortest variant. Compared to the α1, α2, and α5 chains, laminin α4 has a truncated N-terminus Laminin α4 is widely distributed both in adults and during development. It is present in laminin-8 (laminin 411 or alpha4/beta1/gamma1), laminin-9 (laminin 421 or alpha 4/beta2/gamma1), and laminin-14 (laminin 411 or alpha 4/beta1/gamma1).

Unless otherwise apparent from context, reference to laminin α4 or its fragments, domains, or modules includes the natural human amino acid sequences including isoforms and allelic variants thereof. Exemplary human sequences are designated UniProt Number Q16363 and GenBank Accession Numbers NP001098676 and NP001098677 (SEQ ID NOS:1, 2, and 3, respectively). Some antibodies bind to an epitope within the LG1-3 modules of the G domain of laminin α4. The epitope can be in LG1, in LG2, in LG3, or split so that residues forming the epitope come from LG1 and LG2, LG2 and LG3, LG1 and LG3, or all of LG1, LG2, and LG3.

Laminin α4 can bind to both MCAM and integrin α6β1. MCAM (melanoma cell adhesion molecule, also known as CD146 and MUC18) is a 113 kDA cell surface glycoprotein belonging to the immunoglobulin superfamily reported to be involved in cell adhesion and in cohesion of the endothelial monolayer at intercellular junctions in vascular tissue. It has also been reported to promote tumor progression of many cancers, such as solid tumors, including melanoma and prostate cancer. It is known to interact in a homotypic/homophilic manner and may also bind to other ligands. It has a signal peptide, five immunoglobulin-like domains, a transmembrane region, and a short cytoplasmic tail. Lehmann et al., Proc. Nat'l Acad. Sci. USA 86: 9891-9895 (1989). Unless otherwise apparent from context, reference to MCAM or its fragments or domains includes the natural human amino acid sequences including isoforms and allelic variants thereof. An exemplary human sequence is designated UniProt Number P43121 (SEQ ID NO:12).

Integrins are transmembrane receptors that mediate the attachment of a cell to adjacent cells or the extracellular matrix. Integrins are heterodimers composed of two subunits: an alpha subunit and a beta subunit. In mammals, at least eighteen alpha subunits and eight beta subunits have been reported. Through different combinations of alpha and beta subunits, several unique integrins can be generated. Integrins have been reported to have diverse roles in several biological processes including cell migration, cell differentiation, and apoptosis. Their activities have also been reported to regulate the metastatic and invasive potential of tumor cells.

Integrin α6β1 has an alpha 6 subunit (also known as ITGA6, integrin alpha-6, integrin alpha chain 6, CD antigen-like family member F, CD49f, and VLA-6) and a beta 1 subunit (also known as ITGB1, integrin beta-1, integrin beta chain 1, fibronectin receptor subunit beta, glycoprotein IIA, GPIIA, VLA-4 subunit beta, and CD29). Integrin α6β1 has been reported to be involved in cell migration, embryonic development, leukocyte activation, and tumor cell invasiveness. It has also been reported to be a laminin receptor on plateletes, leukocytes, and many epithelial cells. Unless otherwise apparent from context, reference to integrin α6β1, integrin alpha 6, integrin beta 1, or their fragments or domains includes the natural human amino acid sequences including isoforms and allelic variants thereof. An exemplary human sequence for the alpha 6 subunit is designated UniProt Number P23229 (SEQ ID NO:13). An exemplary human sequence for the beta 1 subunit is designated UniProt Number P05556 (SEQ ID NO:14).

### III. Immune Disorders

The above target molecules are involved in various undesirable immune responses.

One category of immune disorders with undesirable immune responses is autoimmune diseases. Autoimmune diseases include systemic autoimmune diseases, organ- or tissue-specific autoimmune diseases, and diseases that exhibit autoimmune-type expressions. In these diseases, the body develops a cellular and/or humoral immune response against one of its own antigens, leading to destruction of that antigen and potentially crippling and/or fatal consequences. The cellular response if present can be B-cell or T-cell or both. TH17 cells, a lineage T helper cells characterized by production of interleukin (IL)-17 and IL-22, have been reported to enter tissues to facilitate pathogenic autoimmune responses, including multiple sclerosis in humans and experimental autoimmune encephalomyelitis (EAE) in mice. See, e.g., Cua et al., Nature 421: 744-748 (2003); Ivonov et al., Cell 126: 1121-1133 (2006). TH17 cells may initiate or propagate an inflammatory response by their specific recruitment to and infiltration of tissue.

Examples of autoimmune diseases include Graves' disease, Hashimoto's thyroiditis, autoimmune polyglandular syndrome, insulin-dependent diabetes mellitus (type 1 diabetes), insulin-resistant diabetes mellitus (type 2 diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barre syndrome, stiff man syndrome, acute rheumatic fever, sympathetic ophthalmia, Goodpasture's syndrome, autoimmune uveitis, temporal arteritis, Bechet's disease, inflammatory bowel diseases, Crohn's disease, ulcerative colitis, primary biliary cirrhosis, autoimmune hepatitis, autoimmune oophoritis, fibromyalgia, polymyositis, dermatomyostis, ankylosing spondylitis, Takayashu arteritis, panniculitis, pemphigoid, vasculitis of unknown origin, anca negative vasculitis, anca positive vasculitis, systemic lupus erythematosus, psoriatic arthritis, rheumatoid arthritis, scleroderma, systemic necrotizing vasculitis, Wegener's granulomatosis, CREST syndrome, antiphospholipid syndrome, Sjogren's syndrome, eosinophilic gastroenteritis, atypical topical dermatitis, cardiomyopathy, post-infectious syndromes, postinfectious endomyocarditis, celiac disease, multiple sclerosis, sarcoidosis, and psoriasis.

Another undesirable immune response is transplant rejection. When allogeneic cells or organs (e.g., skin, kidney, liver, heart, lung, pancreas and bone marrow) are transplanted into a host (i.e., the donor and donee are different individual from the same species), the host immune system is likely to mount an immune response to foreign antigens in the transplant (host-versus-graft disease) leading to destruction of the transplanted tissue. As with autoimmune diseases, TH17 cells have been reported to play a role in transplant rejection. See Heidt et al., Curr. Opin. Organ Transplant 15(4):456-61 (2010).

A related undesirable immune response is the immune response involved in “graft versus host” disease (GVHD). GVHD is a potentially fatal disease that occurs when immunologically competent cells are transferred to an allogeneic recipient. In this situation, the donor's immunocompetent cells may attack tissues in the recipient. Tissues of the skin, gut epithelia, and liver are frequent targets and may be destroyed during the course of GVHD. The disease presents an especially severe problem when immune tissue is being transplanted, such as in bone marrow transplantation, but less severe GVHD has also been reported in other cases as well, including heart and liver transplants. As with autoimmune diseases, TH17 cells have been reported to mediate GVHD. See Carlson et al., Blood 113(6):1365-1374 (2009).

Other immune disorders include allergies, allergic responses, and allergic diseases or disorders. Allergic diseases are characterized by an allergic and/or atopic immunological reaction to an antigen. They are typically associated with chronic inflammation characterized by influx of a large number of eosinophils, accumulation of mast cells, and increased IgE production. Examples of allergic diseases include asthma, chronic obstructive pulmonary disease, allergic rhinitis, allergic contact dermatitis, and atopic dermatitis. Asthma is an inflammatory disorder of the airways characterized by chronic inflammation, airway hyperreactivity, and by symptoms of recurrent wheezing, coughing, and shortness of breath. As with autoimmune diseases, TH17 cells have been reported to play a role in asthma pathogenesis (see Cosmi et al., Allergy 66: 989-998 (2011)) and in allergies and the pathogenesis of allergic diseases (see Oboki et al., Allergology International 57:121-134 (2008)).

### IV. Antibodies

A. Binding Specificity and Functional Properties

The invention provides antibodies binding to epitopes within the laminin α4 protein. More specifically, the invention provides antibodies binding to epitopes within the LG1-3 modules of the G domain of laminin α4. For example, as defined by laminin α4 UniProt sequence Q16363, LG1 (SEQ ID NO:5) includes amino acid positions 833-1035, LG2 (SEQ ID NO:6) includes amino acid positions 1047-1227, LG3 (SEQ ID NO:7) includes amino acid positions 1234-1402, and LG1-3 (SEQ ID NO:8) includes amino acid positions 833-1402. The epitope can be in LG1, in LG2, in LG3, or split so that residues forming the epitope come from LG1 and LG2, LG2 and LG3, LG1 and LG3, or all of LG1, LG2, and LG3. The epitope can be in particular segments within LG1-3, such as segments from laminin α4 UniProt sequence Q16363 ranging from positions 833-883, 884-934, 935-985, 986-1036, 1037-1087, 1088-1138, 1139-1189, 1190-1240, 1241-1291, 1292-1342, and 1343-1402. The epitope can be linear, such as an epitope of, for example, 2-5, 3-5, 3-10, 3-15, 3-20, 5-10, 5-15, or 5-20 contiguous amino acids from LG1, LG2, LG3, LG1-3, LG1-2, LG2-3, or any of the segments or pairs of adjoining segments specified above. The epitope can also be a conformational epitope including, for example, 2-5, 3-5, 3-10, 3-15, 3-20, 5-10, 5-15, or 5-20 non-contiguous amino acids from any combination of LG1, LG2, LG3, LG1-3, and any of the segments specified above.

Antibodies designated 19C12, 1C1, 5Al2, 5B5, and 12D3 are five such exemplary mouse antibodies. These five monoclonal antibodies each specifically bind within the LG1-3 modules of the G domain of laminin α4. These antibodies are further characterized by their lack of significant binding to the LG4-5 modules of the G domain of laminin α4 (e.g., same within experimental error as an irrelevant control antibody, or binding that is at least 2-fold, 3-fold, 4-fold, 5-fold, or 10-fold less (e.g., as measured by a flow cytometric binding assay) than an antibody specific for the LG4-5 modules). Some antibodies are also characterized by their lack of significant binding to other laminin alpha chains, e g , laminin al, laminin α2, laminin α3, and laminin α5 (e.g., same within experimental error as an irrelevant control antibody, or binding that is at least 2-fold, 3-fold, 4-fold, 5-fold, or 10-fold less (e.g., as measured by a flow cytometric binding assay) than an antibody specific for the relevant other laminin alpha chain). Ability to bind to specific proteins, modules, or domains may be demonstrated using exemplary assay formats provided in the examples.

The antibodies are also characterized in that an antibody as a single agent has a capacity to inhibit binding of laminin α4 to MCAM, as shown in Example 2. Preferred antibodies also have the capacity to inhibit binding of laminin α4 to integrin α6β1, as shown in Example 4. Antibodies can also have the capacity to inhibit binding of laminin α4 to other integrins to which laminin α4 can bind, such as integrin ON. Inhibition of binding may be demonstrated in a binding assay in which an antibody of the invention is pre-incubated with recombinant laminin α4 protein, laminin-α4-positive mouse brain tissue, or laminin-α4-displaying cells, after which recombinant MCAM or MCAM-expressing cells or recombinant integrin α6β1 or integrin-α6β1-expressing cells are then assessed for their ability to bind to laminin α4. Exemplary assay formats for showing inhibition are provided in the examples. Optionally, inhibition of a test antibody can be demonstrated in comparison to an irrelevant control antibody not binding within the LG1-3 modules of the G domain of laminin α4 or in comparison to vehicle lacking any antibody.

Some antibodies also have the capacity to inhibit laminin-α4-mediated cell adhesion. An exemplary cell adhesion assay is described in the examples.

Some antibodies also have the capacity to inhibit laminin-α4-induced pAkt activation. An exemplary assay is described in the examples.

Inhibition means an inhibition of at least 10%, 20%, 25%, 30%, 40%, 50%, or 75%, (e.g., 10%-75% or 30%-70%) of binding, cell adhesion and/or other functional activity mediated by laminin α4, either alone or in combination with MCAM, integrin α6β1, or anything else required for any of its functional activities. Inhibition can usually demonstrated when the antibody is present at a concentration of about 20 ug/ml. Some antibodies show inhibition of at least 50% of laminin α4 binding to MCAM, at least 50% of laminin α4 binding to integrin α6β1, or at least 50% of laminin-α4-mediated cell adhesion, preferably cell adhesion mediated by the LG1-3 modules of the G domain of laminin α4.

Some antibodies can inhibit an immune disorder or cancer as shown in an animal model or clinical trial. An exemplary animal model for testing activity against graft versus host disease is a xenographic model utilizing immunodeficient mice receiving human immunocompetent cells, such as the model described in Ito et al., Transplantation 87:1654-1658 (2009). An exemplary animal model of psoriasis is the SCID/psoriasis model described by Villadsen et al., J. Clin. Invest. 112:1571-1580. An exemplary model of multiple sclerosis and T-cell-mediated autoimmune disease in general is the mouse model of experimental autoimmune encephalomyelitis (EAE) described in Flanagan et al., PLoS One 7(7):e40443 (2012). Cell-based assays for particular characteristics of cancer cells, such as proliferation assays, growth assays, survival assays, migration assays, invasion assays, and others, are widely available. Similarly, animal models of cancer in which human cancer cells are injected into an immunodeficient laboratory animal, such as a mouse or rat, or transgenic models in which a laboratory animal expresses a human oncogene or has a knocked out tumor suppressor gene, are widely available.

Some antibodies bind to the same or overlapping epitope as an antibody designated 19C12, 1C1, 5Al2, 5B5, or 12D3. The sequences of the heavy and light chain mature variable regions of these antibodies are designated SEQ ID NOS:15 and 16, 25 and 26, 35/36 and 37, 50 and 51, and 60/61 and 62, respectively. Another version of the heavy chain mature variable region of 1C1 is SEQ ID NO:141. Other antibodies having such a binding specificity can be produced by immunizing mice with laminin α4, or a portion thereof including the desired epitope, and screening resulting antibodies for binding to the LG1-3 modules of the G domain of laminin α4, optionally in competition with 19C12, 1C1, 5Al2, 5B5, or 12D3. Antibodies identified by such assays can then be screened for ability to specifically bind to the LG1-3 modules but not the LG4-5 modules of the G domain of laminin α4 as described in the examples or otherwise. Antibodies can also be screened for ability to inhibit binding of laminin α4 to MCAM as described in the examples or otherwise. Antibodies can also be screened for ability to inhibit binding of laminin α4 to integrin α6β1 as described in the examples or otherwise. Antibodies can also be screened for ability to inhibit laminin-α4-mediated cell adhesion as described in the examples or otherwise.

Antibodies binding to an epitope that includes one or more specified residues can be generated by immunizing with a fragment of laminin α4 that includes these one or more residues. The fragment can, for example, have no more than 100, 50, 25, 10 or 5 contiguous amino acids from SEQ ID NO:8. Such fragments usually have at least 5, 6, 7, 8 or 9 contiguous residues of SEQ ID NO:8. The fragments can be linked to a carrier that helps elicit an antibody response to the fragment and/or be combined with an adjuvant that helps elicit such a response. Alternatively, antibodies binding to a desired residue can be obtained by immunizing with a full-length laminin α4 (SEQ ID NO:1) or the full-length G domain of laminin α4 (SEQ ID NO:4) or the LG1-3 modules of the G domain of laminin α4 (SEQ ID NO:8) or fragments of any of these. Such antibodies can then be screened for differential binding to versions of laminin α4 containing different LG modules of the G domain, such as LG1-3, LG1-5, LG4-5, LGde3, LGde1, LGde2, LG1, LG2, or LG3 (SEQ ID NOS:8, 4, 11, 124, 125, 126, 5, 6, and 7 respectively), or differential binding to wild type laminin α4 compared with mutants of specified residues. The screen against versions of laminin α4 with different LG modules of the G domain maps antibody binding to certain LG modules within the G domain of laminin α4. The screen against mutants more precisely defines the binding specificity to allow identification of antibodies whose binding is inhibited by mutagenesis of particular residues and which are likely to share inhibitor properties of other exemplified antibodies.

Human antibodies having the binding specificity of a selected murine antibody (e.g., 19C12, 1C1, 5Al2, 5B5, or 12D3) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for the LG1-3 modules of the G domain of laminin α4 (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for the LG1-3 modules of the G domain of laminin α4 are selected. The resulting antibodies usually have the same or similar epitope specificity as the murine starting material.

Other antibodies can be obtained by mutagenesis of cDNA encoding the heavy and light chains of an exemplary antibody, such as 19C12, 1C1, 5Al2, 5B5, or 12D3. Monoclonal antibodies that are at least 70%, 80%, 90%, 95%, 96%, 97%, 98%, or 99% identical to 19C12, 1C1, 5Al2, 5B5, or 12D3 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one or all six CDR(s) as defined by Kabat that are 90%, 95%, 99% or 100% identical to corresponding CDRs of 19C12, 1C1, 5Al2, 5B5, or 12D3 are also included.

The invention also provides antibodies having some or all (e.g., 3, 4, 5, and 6) CDRs entirely or substantially from 19C12, 1C1, 5Al2, 5B5, or 12D3. Such antibodies can include a heavy chain variable region that has at least two, and usually all three, CDRs entirely or substantially from the heavy chain variable region of 19C12, 1C1, 5Al2, 5B5, or 12D3 and/or a light chain variable region having at least two, and usually all three, CDRs entirely or substantially from the light chain variable region of 19C12, 1C1, 5Al2, 5B5, or 12D3. The antibodies can include both heavy and light chains. A CDR is substantially from a corresponding 19C12, 1C1, 5Al2, 5B5, or 12D3 CDR when it contains no more than 4, 3, 2, or 1 substitutions, insertions, or deletions, except that CDRH2 (when defined by Kabat) can have no more than 6, 5, 4, 3, 2, or 1 substitutions, insertions, or deletions. Such antibodies can have at least 70%, 80%, 90%, 95%, 96%, 97%, 98%, or 99% identity to 19C12, 1C1, 5Al2, 5B5, or 12D3 in the amino acid sequence of the mature heavy and/or light chain variable regions and maintain their functional properties, and/or differ from 19C12, 1C1, 5Al2, 5B5, or 12D3 by a small number of functionally inconsequential amino acid substitutions (e.g., conservative substitutions), deletions, or insertions.

B. Non-Human Antibodies

The production of other non-human antibodies, e.g., murine, guinea pig, primate, rabbit or rat, against the LG1-3 modules of the G domain of laminin α4 can be accomplished by, for example, immunizing the animal with laminin α4 or a fragment thereof. See Harlow & Lane, Antibodies, A Laboratory Manual (CSHP NY, 1988) (incorporated by reference for all purposes). Such an immunogen can be obtained from a natural source, by peptide synthesis, or by recombinant expression. Optionally, the immunogen can be administered fused or otherwise complexed with a carrier protein. Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to the LG1-3 modules of the G domain of laminin α4. Such screening can be accomplished by determining binding of an antibody to a collection of laminin α4 variants, such as laminin α4 variants containing the LG1-3 modules of the G domain, the LG1-5 modules of the G domain, and the LG4-5 modules of the G domain, and determining which laminin α4 variants bind to the antibody. Binding can be assessed, for example, by Western blot, FACS or ELISA.

C. Humanized Antibodies

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs) from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

An example of an acceptor sequence for the heavy chain is the human mature heavy chain variable region with NCBI accession code BAC01530.1 (SEQ ID NO:75). This acceptor sequence includes two CDRs having the same canonical form as mouse 19C12 heavy chain. Examples of acceptor sequences for the light chain are the human mature light chain variable regions with NCBI accession codes ABA71367.1 and ABI75162.1 (SEQ ID NOS:76 and 77, respectively). These acceptor sequences include three CDRs having the same canonical form as mouse 19C12 light chain.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to a CDR region or within a CDR as defined by Chothia but not Kabat,
(3) otherwise interacts with a CDR region (e.g. is within about 6 Å of a CDR region), (e.g., identified by modeling the light or heavy chain on the solved structure of a homologous known immunoglobulin chain), or
(4) is a residue participating in the VL-VH interface.

Framework residues from classes (1) through (3) as defined by Queen, U.S. Pat. No. 5,530,101, are sometimes alternately referred to as canonical and vernier residues. Framework residues that help define the conformation of a CDR loop are sometimes referred to as canonical residues (Chothia & Lesk, J. Mol. Biol. 196:901-917 (1987); Thornton & Martin, J. Mol. Biol. 263:800-815 (1996)). Framework residues that support antigen-binding loop conformations and play a role in fine-tuning the fit of an antibody to antigen are sometimes referred to as vernier residues (Foote & Winter, J. Mol. Viol 224:487-499 (1992)).

Other framework residues that are candidates for substitution are residues creating a potential glycosylation site. Still other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins.

Exemplary humanized antibodies are humanized forms of the mouse 19C12 antibody, designated Hu19C12. The mouse antibody comprises mature heavy and light chain variable regions having amino acid sequences comprising SEQ ID NO:15 and SEQ ID NO:16, respectively. The invention provides three exemplified humanized mature heavy chain variable regions: Hu19C12VHv1 (H1; SEQ ID NO:80), Hu19C12VHv2 (H2; SEQ ID NO:81), and Hu19C12VHv3 (H3; SEQ ID NO:82). The invention further provides six exemplified human mature light chain variable regions: Hu19C12VLv1 (L1; SEQ ID NO:83), Hu19C12VLv2 (L2; SEQ ID NO:84), Hu19C12VLv3 (L3; SEQ ID NO:85), Hu19C12VLv4 (L4; SEQ ID NO:86), Hu19C12VLv5 (L5; SEQ ID NO:87), and Hu19C12VLv6 (L6; SEQ ID NO:88).

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, and other reasons, the following 24 variable region framework positions were considered as candidates for substitutions in the six exemplified human mature light chain variable regions and the three exemplified human mature heavy chain variable regions, as further specified in the examples: L1 (D1N), L9 (L9A), L22 (N22S), L49 (S49C), L68 (G68R), L76 (S76D), L77 (S77P), L78 (L78V), L79 (Q79E), L85 (L85T), L100 (Q100A), H1 (Q1E), H11 (V11L), H12 (K12V), H16 (S16A), H20 (V20I), H27 (G27Y), H28 (T28A), H38 (R38K), H43 (Q43E), H48 (M48I), H69 (I69L), H91 (Y91F), and H108 (M108T). Position L49 can also be substituted with other amino acids, such as I, T, A, M, Q, or E, which may confer improved stability relative to substitution to a cysteine.

Here, as elsewhere, the first-mentioned residue is the residue of a humanized antibody formed by grafting Kabat CDRs into a human acceptor framework, and the second-mentioned residue is a residue being considered for replacing such residue. Thus, within variable region frameworks, the first mentioned residue is human, and within CDRs, the first mentioned residue is mouse.

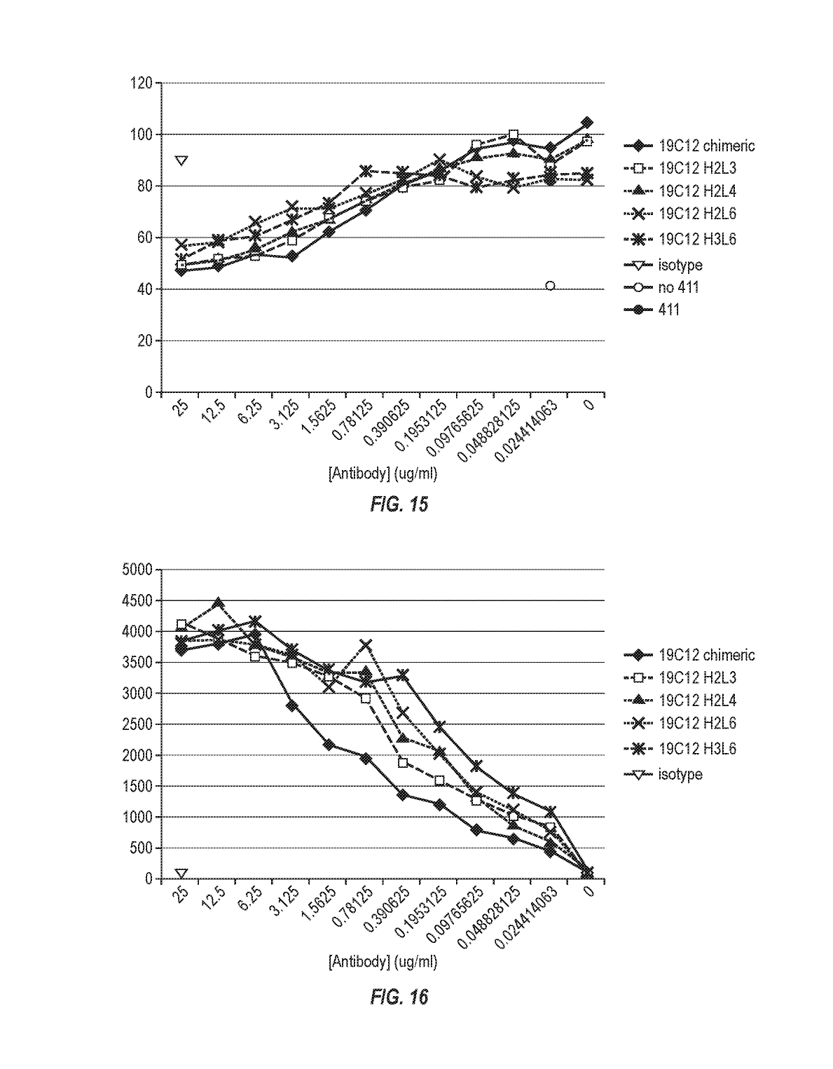

Exemplified antibodies include any permutations or combinations of the exemplified mature heavy and light chain variable regions (e.g., VHv1/VLv1 or H1L1, VHv1/VLv2 or H1L2, VHv1/VLv3 or H1L3, VHv1/VLv4 or H1L4, VHv1/VLv5 or H1L5, VHv1/VLv6 or H1L6, VHv2/VLv1 or H2L1, VHv2/VLv2 or H2L2, VHv2/VLv3 or H2L3, VHv2/VLv4 or H2L4, VHv2/VLv5 or H2L5, VHv2/VLv6 or H2L6, VHv3/VLv1 or H3L1, VHv3/VLv2 or H3L2, VHv3/VLv3 or H3L3, VHv3/VLv4 or H3L4, VHv3/VLv5 or H3L5, and VHv3/VLv6) or H3L6). For example, the H2L3 antibody, which includes 8 heavy chain backmutations or other mutations and 11 light chain backmutations as described below, binds to laminin α4 and inhibits MCAM binding to laminin α4 at a level that is substantially the same as a chimeric 19C12 antibody (see FIGS. 15-18). Comparable results are seen with the H2L4, H2L6, and H3L6 antibodies (see FIGS. 15-18).

The invention provides variants of the H2L3 humanized 19C12 antibody in which the humanized mature heavy chain variable region shows at least 90%, 95%, 96%, 97%, 98%, or 99% identity to H2 (SEQ ID NO:81) and the humanized mature light chain variable region shows at least 90%, 95%, 96%, 97%, 98%, or 99% identity to L3 (SEQ ID NO:85). In some such antibodies at least 1, 2, 3, 4, 5, 6, 7, 8, 9, 10, 11, 12, 13, 14, 15, 16, 17, 18, or all 19 of the backmutations or other mutations in H2L3 are retained. The invention also provides variants of the H3L6 humanized 19C12 antibody in which the humanized mature heavy chain variable region shows at least 90%, 95%, 96%, 97%, 98%, or 99% identity to H3 (SEQ ID NO:82) and the humanized mature light chain variable region shows at least 90%, 95%, 96%, 97%, 98%, or 99% identity to L6 (SEQ ID NO:88). In some such antibodies at least 1, 2, 3, 4, 5, 6, 7, 8, 9, 10, 11, 12, 13, 14, 15, or all 16 of the backmutations or other mutations in H3L6 are retained. In some antibodies, at least one of positions H11, H12, H16, H27, H28, H48, H91, and H108 in the Vh region is occupied by L, V, A, Y, A, I, F, and T, respectively. In some antibodies, positions H11, H12, H16, H27, H28, H48, H91, and H108 in the Vh region are occupied by L, V, A, Y, A, I, F, and T, respectively. In some antibodies, at least one of positions H1, H20, H38, H43, and H69 in the Vh region is occupied by E, I, K, E, and L, respectively. In some antibodies, positions H20, H38, H43, and H69 in the Vh region are occupied by I, K, E, and L, respectively, such as in version H1. In some antibodies, position H1 in the Vh region is occupied by E, such as in version H3. In some antibodies, at least one of positions L9, L22, and L85 in the Vk region is occupied by A, S, and T, respectively. In some antibodies, positions L9, L22, and L85 in the Vk region are occupied by A, S, and T, respectively. In some antibodies, at least one of positions L1, L49, L68, L76, L77, L78, L79, and L100 in the Vk region is occupied by N, C, R, D, P, V, E, and A, respectively. In some antibodies, positions L1, L49, and L68 in the Vk region are occupied by N, C, and R, respectively, such as in version L1. In some antibodies, position L1 in the Vk region is occupied by N, such as in version L2. In some antibodies, positions L1, L49, L68, L76, L77, L78, L79, and L100 in the Vk region are occupied by N, C, R, D, P, V, E, and A, respectively, such as in version L3. In some antibodies, positions L1, L77, L78, L79, and L100 in the Vk region are occupied by N, P, V, E, and A, respectively, such as in version L4. In some antibodies, position L77 in the Vk region is occupied by P, such as in version L5. In some antibodies, positions L77, L78, L79, and L100 in the Vk region are occupied by P, V, E, and A, respectively, such as in version L6. The CDR regions of such humanized antibodies can be identical or substantially identical to the CDR regions of H2L3, which are the same as those of the mouse donor antibody. The CDR regions can be defined by any conventional definition (e.g., Chothia) but are preferably as defined by Kabat.

The invention also provides variants of the other exemplified Hu19C12 antibodies. Such variants have mature light and heavy chain variable regions showing at least 90%, 95%, 96%, 97%, 98%, or 99% sequence identity to the mature light and heavy chain variable regions of the exemplified humanized 19C12 H1L1, H1L2, H1L3, H1L4, H1L5, H1L6, H2L1, H2L2, H2L4, H2L5, H2L6, H3L1, H3L2, H3L3, H3L4, H3L5, or H3L6 antibodies. The CDR regions of such humanized antibodies can be identical or substantially identical to those of the mouse donor antibody. The CDR regions can be defined by any conventional definition (e.g., Chothia) but are preferably defined by Kabat. Other such variants typically differ from the sequences of the exemplified Hu19C12 antibodies by a small number (e.g., typically no more than 1, 2, 3, 5, 10, or 15) of replacements, deletions or insertions. Such differences are usually in the framework but can also occur in the CDRs.

A possibility for additional variation in humanized 19C12 variants is additional backmutations in the variable region frameworks. Many of the framework residues not in contact with the CDRs in the humanized mAb can accommodate substitutions of amino acids from the corresponding positions of the donor mouse mAb or other mouse or human antibodies, and even many potential CDR-contact residues are also amenable to substitution. Even amino acids within the CDRs may be altered, for example, with residues found at the corresponding position of the human acceptor sequence used to supply variable region frameworks. In addition, alternate human acceptor sequences can be used, for example, for the heavy and/or light chain.

If different acceptor sequences are used, one or more of the backmutations recommended above may not be performed because the corresponding donor and acceptor residues are already the same without backmutations.

Preferably, replacements or backmutations in Hu19C12 (whether or not conservative) have no substantial effect on the binding affinity or potency of the humanized mAb, that is, its ability to inhibit binding of laminin α4 to MCAM and/or integrin α6β1 (e.g., the potency in some or all of the assays described in the present examples of the variant humanized 19C12 antibody is essentially the same, i.e., within experimental error, as that of murine 19C12 or H2L3).

D. Chimeric and Veneered Antibodies

The invention further provides chimeric and veneered forms of non-human antibodies, particularly the 19C12, 1C1, 5Al2, 5B5, or 12D3 antibodies of the examples.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of the 19C12 antibody are included in the invention.

E. Human Antibodies

Human antibodies against the LG1-3 modules of the G domain of laminin α4 are provided by a variety of techniques described below. Some human antibodies are selected by competitive binding experiments, by the phage display method of Winter, above, or otherwise, to have the same epitope specificity as a particular mouse antibody, such as one of the mouse monoclonal antibodies described in the examples. Human antibodies can also be screened for a particular epitope specificity by using only a fragment of laminin α4, such as a laminin α4 variant containing only the LG1-3 modules of the G domain, as the target antigen, and/or by screening antibodies against a collection of laminin α4 variants, such as laminin α4 variants containing the LG1-3 modules of the G domain, the LG1-5 modules of the G domain, and the LG4-5 modules of the G domain.

Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, US 5,545,806, Nature 148, 1547-1553 (1994), Nature Biotechnology 14, 826 (1996), Kucherlapati, WO 91/10741 (1991)) and phage display methods (see, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332).

F. Selection of Constant Region

The heavy and light chain variable regions of chimeric, humanized (including veneered), or human antibodies can be linked to at least a portion of a human constant region. The choice of constant region depends, in part, on whether antibody-dependent complement and/or cellular mediated cytotoxicity is desired. For example, human isotypes IgG1 and IgG3 have complement-mediated cytotoxicity and human isotypes IgG2 and IgG4 do not. Human IgG1 and IgG3 also induce stronger cell mediated effector functions than human IgG2 and IgG4. A human IgG1 constant region suitable for inclusion in the antibodies can have the sequence of SEQ ID NO:89. The C-terminal lysine of SEQ ID NO:89 can be omitted, in which case the IgG1 constant region has the amino acid sequence of SEQ ID NO:140. Light chain constant regions can be lambda or kappa. A human kappa light chain constant region suitable for inclusion in the antibodies can have the sequence of SEQ ID NO:139. SEQ ID NO:139 can be encoded by the nucleic acid sequence of SEQ ID NO:149. The N-terminal arginine of SEQ ID NO:139 can be omitted, in which case the kappa light chain constant region has the amino acid sequence of SEQ ID NO:90. SEQ ID NO: 90 can be encoded by the nucleic acid sequence of SEQ ID NO:151. Antibodies can be expressed as tetramers containing two light and two heavy chains, as separate heavy chains, light chains, as Fab, Fab′, F(ab′)2, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Thus, for example, another heavy chain constant region is of the IgG1 G1m3 allotype and has the amino acid sequence of SEQ ID NO:138. SEQ ID NO:138 can be encoded by the nucleic acid sequence of SEQ ID NO:148. Another heavy chain constant region of the IgG1 G1m3 allotype has the amino acid sequence of SEQ ID NO:150. Reference to a human constant region includes a constant region with any natural allotype or any permutation of residues occupying polymorphic positions in natural allotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering) for increasing the half-life of an antibody. Substitution at any of positions 234, 235, 236 and/or 237 reduces affinity for Fcγ receptors, particularly FcγRI receptor (see, e.g., U.S. Pat. No. 6,624,821). An alanine substitution at positions 234, 235, and 237 of human IgG1 can be used for reducing effector functions. Optionally, positions 234, 236 and/or 237 in human IgG2 are substituted with alanine and position 235 with glutamine See, e.g., U.S. Pat. No. 5,624,821. In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine, such as in SEQ ID NO:150. In some antibodies, the isotype is human IgG2 or IgG4.

G. Expression of Recombinant Antibodies

A number of methods are known for producing chimeric and humanized antibodies using an antibody-expressing cell line (e.g., hybridoma). For example, the immunoglobulin variable regions of antibodies can be cloned and sequenced using well known methods. In one method, the heavy chain variable VH region is cloned by RT-PCR using mRNA prepared from hybridoma cells. Consensus primers are employed to the VH region leader peptide encompassing the translation initiation codon as the 5′ primer and a g2b constant regions specific 3′ primer. Exemplary primers are described in U.S. patent publication US 2005/0009150 by Schenk et al. (hereinafter “Schenk”). The sequences from multiple, independently derived clones can be compared to ensure no changes are introduced during amplification. The sequence of the VH region can also be determined or confirmed by sequencing a VH fragment obtained by 5′ RACE RT-PCR methodology and the 3′ g2b specific primer.

The light chain variable VL region can be cloned in an analogous manner. In one approach, a consensus primer set is designed for amplification of VL regions using a 5′ primer designed to hybridize to the VL region encompassing the translation initiation codon and a 3′ primer specific for the Ck region downstream of the V-J joining region. In a second approach, 5′RACE RT-PCR methodology is employed to clone a VL encoding cDNA. Exemplary primers are described in Schenk, supra. The cloned sequences are then combined with sequences encoding human (or other non-human species) constant regions. Exemplary sequences encoding human constant regions include SEQ ID NO:89, which encodes a human IgG1 constant region, and SEQ ID NO:90, which encodes a human kappa light chain constant region.

In one approach, the heavy and light chain variable regions are re-engineered to encode splice donor sequences downstream of the respective VDJ or VJ junctions and are cloned into a mammalian expression vector, such as pCMV-hγ1 for the heavy chain and pCMV-Mcl for the light chain. These vectors encode human γ1 and Ck constant regions as exonic fragments downstream of the inserted variable region cassette. Following sequence verification, the heavy chain and light chain expression vectors can be co-transfected into CHO cells to produce chimeric antibodies. Conditioned media is collected 48 hours post-transfection and assayed by western blot analysis for antibody production or ELISA for antigen binding. The chimeric antibodies are humanized as described above.

Chimeric, veneered, humanized, and human antibodies are typically produced by recombinant expression. Recombinant polynucleotide constructs typically include an expression control sequence operably linked to the coding sequences of antibody chains, including naturally associated or heterologous expression control elements, such as a promoter. The expression control sequences can be promoter systems in vectors capable of transforming or transfecting eukaryotic or prokaryotic host cells. Once the vector has been incorporated into the appropriate host, the host is maintained under conditions suitable for high level expression of the nucleotide sequences and the collection and purification of the crossreacting antibodies.

These expression vectors are typically replicable in the host organisms either as episomes or as an integral part of the host chromosomal DNA. Commonly, expression vectors contain selection markers, e.g., ampicillin resistance or hygromycin resistance, to permit detection of those cells transformed with the desired DNA sequences.

E. coli is one prokaryotic host useful for expressing antibodies, particularly antibody fragments. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NSO. The cells can be nonhuman Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. No. 5,741,957, U.S. Pat. No. 5,304,489, U.S. Pat. No. 5,849,992). Suitable transgenes include coding sequences for light and/or heavy chains operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics, or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes or can be incorporated into the genome of embryonic stem cells, and the nuclei of such cells transferred into enucleated oocytes.

Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivities above 50 pg or 100 pg per cell per day, which correspond to product titers of greater than 7.5 g/L culture, can be used. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, antibodies can be purified according to standard procedures of the art, including protein A capture, HPLC purification, column chromatography, gel electrophoresis, and the like (see generally, Scopes, Protein Purification (Springer-Verlag, NY, 1982)).

Methodology for commercial production of antibodies can be employed, including codon optimization, selection of promoters, selection of transcription elements, selection of terminators, serum-free single cell cloning, cell banking, use of selection markers for amplification of copy number, CHO terminator, or improvement of protein titers (see, e.g., U.S. Pat. No. 5,786,464, U.S. Pat. No. 6,114,148, U.S. Pat. No. 6,063,598, U.S. Pat. No. 7,569,339, W02004/050884, W02008/012142, W02008/012142, W02005/019442, W02008/107388, and W02009/027471, and U.S. Pat. No. 5,888,809).

H. Nucleic Acids

The invention further provides nucleic acids encoding any of the heavy and light chains described above (e.g., SEQ ID NOS: 91-92, 95-96, 99-101, 105-106, 109-111, and 115-123). SEQ ID NOS:146, 148, 149, and 151 are additional examples of nucleic acids encoding heavy and light chains described above. Typically, the nucleic acids also encode a signal peptide fused to the mature heavy and light chains (e.g., signal peptides having amino acid sequences of SEQ ID NOS:17, 27, 38, 39, 52, 63, and 64 (heavy chain) and 18, 28, 40, 53, and 65 (light chain), that can be encoded by SEQ ID NOS:93, 97, 102, 103, 107, 112, and 113, respectively (heavy chain), and 94, 98, 104, 108, and 114, respectively (light chain)). An additional example of a signal peptide (heavy chain) has the amino acid sequence of SEQ ID NO:142 and can be encoded by SEQ ID NO:147. Coding sequences of nucleic acids can be operably linked with regulatory sequences to ensure expression of the coding sequences, such as a promoter, enhancer, ribosome binding site, transcription termination signal, and the like. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

I. Conjugated Antibodies

Conjugated antibodies that specifically bind to the LG1-3 modules of the G domain of laminin α4 can be useful in targeting cancer or tumor cells for destruction, targeting cells involved in autoimmune diseases, or suppressing various undesirable immune responses. Such antibodies can also be useful in targeting any other diseases mediated at least in part by expression of the LG1-3 modules of the G domain of laminin α4. For example, such antibodies can be conjugated with other therapeutic moieties, other proteins, other antibodies, and/or detectable labels. See WO 03/057838; U.S. Pat. No. 8,455,622. Such therapeutic moieties can be any agent that can be used to treat, combat, ameliorate, prevent, or improve an unwanted condition or disease in a patient, such as a cancer, an autoimmune disease, or an undesirable immune response. Therapeutic moieties can include cytotoxic agents, cytostatic agents, radiotherapeutic agents, immunomodulators, or any biologically active agents that facilitate or enhance the activity of the antibody. A cytotoxic agent can be any agent that is toxic to a cell. A cytostatic agent can be any agent that inhibits cell proliferation. An immunomodulator can be any agent that stimulates or inhibits the development or maintenance of an immunologic response. A radiotherapeutic agent can be any molecule or compound that emits radiation. If such therapeutic moieties are coupled to a laminin-α4-specific antibody, such as the antibodies described herein, the coupled therapeutic moieties will have a specific affinity for laminin-α4-expressing cells or cells expressing laminin-α4 binding partners, such as MCAM-expressing cells, over other cells. Consequently, administration of the conjugated antibodies directly targets such cells with minimal effects on other surrounding cells and tissue. This can be particularly useful for therapeutic moieties that are too toxic to be administered on their own. In addition, smaller quantities of the therapeutic moieties can be used.

Some such antibodies can be modified to act as immunotoxins. See, e.g., U.S. Pat. No. 5,194,594. For example, ricin, a cellular toxin derived from plants, can be coupled to antibodies by using the bifunctional reagents S-acetylmercaptosuccinic anhydride for the antibody and succinimidyl 3-(2-pyridyldithio)propionate for ricin. See Pietersz et al., Cancer Res. 48(16):4469-4476 (1998). The coupling results in loss of B-chain binding activity of ricin, while impairing neither the toxic potential of the A-chain of ricin nor the activity of the antibody. Similarly, saporin, an inhibitor of ribosomal assembly, can be coupled to antibodies via a disulfide bond between chemically inserted sulfhydryl groups. See Polito et al., Leukemia 18:1215-1222 (2004).

Some such antibodies can be linked to radioisotopes. Examples of radioisotopes include, for example, yttrium⁹⁰ (90Y), indium¹¹¹ (111In), ¹³¹I, ⁹⁹mTc, radiosilver-111, radiosilver-199, and Bismuth²¹³. Linkage of radioisotopes to antibodies may be performed with conventional bifunction chelates. For radiosilver-11 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1:S91-S95 (2001).

Some such antibodies can be linked to other therapeutic moieties. Such therapeutic moieties can be, for example, cytotoxic or cytostatic. For example, antibodies can be conjugated with toxic chemotherapeutic drugs such as maytansine, geldanamycin, tubulin inhibitors such as tubulin binding agents (e.g., auristatins), or minor groove binding agents such as calicheamicin. Other representative therapeutic moieties include agents known to be useful for treatment, management, or amelioration of a cancer or an undesirable immune response (e.g., an autoimmune disease) or symptoms of a cancer or an undesirable immune response (e.g., an autoimmune disease). Examples of such therapeutic agents are disclosed elsewhere herein.

Antibodies can also be coupled with other proteins. For example, antibodies can be coupled with Fynomers. Fynomers are small binding proteins (e.g., 7 kDa) derived from the human Fyn SH3 domain. They can be stable and soluble, and they can lack cysteine residues and disulfide bonds. Fynomers can be engineered to bind to target molecules with the same affinity and specificity as antibodies. They are suitable for creating multi-specific fusion proteins based on antibodies. For example, Fynomers can be fused to N-terminal and/or C-terminal ends of antibodies to create bi- and tri-specific FynomAbs with different architectures. Fynomers can be selected using Fynomer libraries through screening technologies using FACS, Biacore, and cell-based assays that allow efficient selection of Fynomers with optimal properties. Examples of Fynomers are disclosed in Grabulovski et al., J. Biol. Chem. 282:3196-3204 (2007); Bertschinger et al., Protein Eng. Des. Sel. 20:57-68 (2007); Schlatter et al., MAbs. 4:497-508 (2011); Banner et al., Acta. Crystallogr. D. Biol. Crystallogr. 69(Pt6):1124-1137 (2013); and Brack et al., Mol. Cancer Ther. 13:2030-2039 (2014).

The antibodies disclosed herein can also be coupled or conjugated to one or more other antibodies (e.g., to form antibody heteroconjugates). Such other antibodies can bind to different epitopes within the LG1-3 modules of the G domain of laminin α4 or can bind to a different target antigen.

Antibodies can also be coupled with a detectable label. Such antibodies can be used, for example, for diagnosing a cancer or an undesirable immune response (e.g., an autoimmune disease), for monitoring progression of a cancer or an undesirable immune response (e.g., an autoimmune disease), and/or for assessing efficacy of treatment. Such antibodies can be useful for performing such determinations in subjects having or being susceptible to a cancer or an undesirable immune response (e.g., an autoimmune disease), or in appropriate biological samples obtained from such subjects. Representative detectable labels that may be coupled or linked to an antibody include various enzymes, such as horseradish peroxidase, alkaline phosphatase, beta-galactosidase, or acetylcholinesterase; prosthetic groups, such streptavidin/biotin and avidin/biotin; fluorescent materials, such as umbelliferone, fluorescein, fluorescein isothiocyanate, rhodamine, dichlorotriazinylamine fluorescein, dansyl chloride or phycoerythrin; luminescent materials, such as luminol; bioluminescent materials, such as luciferase, luciferin, and aequorin; radioactive materials, such as radiosilver-111, radiosilver-199, Bismuth²¹³, iodine (131I, ¹²⁵I, ¹²³I, ¹²¹I), carbon (¹⁴C), sulfur (⁵_{S}), tritium (³_{H}), indium (¹¹⁵In, ¹¹³In, ¹¹²In, 111^{In},), technetium (⁹⁹Tc), thallium (²⁰¹Ti), gallium (⁶⁸Ga, ^{δ}Ga), palladium (¹⁰³Pd), molybdenum (⁹⁹Mo), xenon (¹³³Xe) , fluorine (¹⁸F), ¹⁵³Sm, ¹⁷⁷Lu, ¹⁵⁹Gd, ¹⁴⁹Pm, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies; nonradioactive paramagnetic metal ions; and molecules that are radiolabelled or conjugated to specific radioisotopes.

Therapeutic moieties, other proteins, other antibodies, and/or detectable labels may be coupled or conjugated, directly or indirectly through an intermediate (e.g., a linker), to a murine, chimeric, veneered, or humanized antibody using techniques known in the art. See e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985); and Thorpe et al., Immunol. Rev., 62:119-58 (1982). Suitable linkers include, for example, cleavable and non-cleavable linkers. Different linkers that release the coupled therapeutic moieties, proteins, antibodies, and/or detectable labels under acidic or reducing conditions, on exposure to specific proteases, or under other defined conditions can be employed.

V. Therapeutic Applications

The antibodies or other antagonists of the invention can be used for suppressing various undesirable immune responses, preferably those involving infiltration of MCAM-expressing cells, and more preferably infiltration of TH17 cells, to a site of inflammation. The location of laminin α4 in the endothelial basement membrane provides evidence of it functioning by augmenting adhesion of TH17 cells attempting endothelial penetration into a tissue, or serving as an adhesion-based gating system to signal appropriate entry mechanisms. As demonstrated in the examples, binding of MCAM to laminin α4 can contribute to this process, either alone or in conjunction with binding of integrin α6β1 to laminin α4.

Several categories of immune disorders characterized by undesirable immune responses are described in Section III. For example, one immune disorder treatable by antibodies of the invention is transplant rejection. Particularly, the antibodies are useful to block alloantigen-induced immune responses in the donee. Another immune disorder treatable by the antibodies of the invention is GVHD. Another immune disorder treatable by the antibodies of the invention is the category of autoimmune diseases, such as diabetes, Crohn's disease, ulcerative colitis, multiple sclerosis, stiff man syndrome, rheumatoid arthritis, myasthenia gravis, systemic lupus erythematosus, celiac disease, psoriasis, psoriatic arthritis, sarcoidosis, ankylosing spondylitis, Sjogren's syndrome, and uveitis. Other immune disorders treatable by the antibodies of the invention include allergies, allergic responses, and allergic diseases, such as asthma and allergic contact dermatitis.

Other disorders treatable by antibodies of the invention include cancers. Cancers can be hematopoietic malignancies or solid tumors, i.e., masses of cells that result from excessive cell growth or proliferation, either benign or malignant, including pre-cancerous legions. Cancers can be benign, malignant, or metastatic. Metastatic cancer refers to a cancer that has spread from the place where it first started to another place in the body. Tumors formed by metastatic cancer cells are called a metastatic tumor or a metastasis, which is a term also used to refer to the process by which cancer cells spread to other parts of the body. In general, metastatic cancer has the same name and same type of cancer cells as the original, or primary, cancer. Examples of cancer include solid tumors, such as melanoma, carcinoma, blastoma, and sarcoma. Cancers also include hematologic malignancies, such as leukemia or lymphoid malignancies, such as lymphoma. More particular examples of such cancers include squamous cell cancer, lung cancer, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The antibodies can be used for treating or effecting prophylaxis of a cancer in a patient having or at risk for the cancer. In some instances the patient has a brain cancer or another type of CNS or intracranial tumor. For example, the patient can have an astrocytic tumor (e.g., astrocytoma, anaplastic astrocytoma, glioblastoma, pilocytic astrocytoma, subependymal giant cell astrocytoma, pleomorphic xanthoastrocytoma), oligodendroglial tumor (e.g., oligodendroglioma, anaplastic oligodendroglioma), ependymal cell tumor (e.g., ependymoma, anaplastic ependymoma, myxopapillary ependymoma, subependymoma), mixed glioma (e.g., mixed oligoastrocytoma, anaplastic oligoastrocytoma), neuroepithelial tumor of uncertain origin (e.g., polar spongioblastoma, astroblastoma, gliomatosis cerebri), tumor of the choroid plexus (e.g., choroid plexus papilloma, choroid plexus carcinoma), neuronal or mixed neuronal-glial tumor (e.g., gangliocytoma, dyplastic gangliocytoma of cerebellum, ganglioglioma, anaplastic ganglioglioma, desmoplastic infantile ganglioma, central neurocytoma, dysembryoplastic neuroepithelial tumor, olfactory neuroblastoma), pineal parenchyma tumor (e.g., pineocytoma, pineoblastoma, mixed pineocytoma/pineoblastoma), or tumor with mixed neuroblastic or glioblastic elements (e.g., medulloepithelioma, medulloblastoma, neuroblastoma, retinoblastoma, ependymoblastoma). In some instances, the patient has melanoma, glioma, glioblastoma, lung cancer, or breast cancer. Treatment can include inhibiting growth and/or metastasis of a cancer. In some instances, the patient has or is at risk of metastatic cancer. In some instances, the metastatic cancer can be prostate cancer, lung cancer, or pancreatic cancer. The invention is particularly amenable to treating cancers in which the LG1-3 modules of the G domain of laminin α4 play a role in cell adhesion. Binding of an antibody to the LG1-3 modules of the G domain of laminin α4 can affect invasive or metastatic capabilities of the cancer. Such binding can also affect signaling mechanisms involved in cell proliferation, growth, resisting cell death, angiogenesis, or other characteristics of cancers. In some instances, the antibodies disrupt or inhibit angiogenesis by altering endothelial D114/Notch signaling. In some cases, the disruption or inhibition of angiogenesis by the antibodies involves disrupting the interaction between laminin α4 and integrins, such as integrins comprising integrin α2, integrin α6, or integrin β1. The antibodies can also inhibit tumor growth via inhibiting Akt activation and subsequent cell survival/proliferation signaling.

Antibody-drug conjugates can have additional mechanisms of action including the cytotoxic or cytostatic effect of the linked agent, typically after uptake within a cancer cell or other targeted cell. Antibody-drug conjugates may also induce tumor-associated macrophage toxicity.

Other disorders treatable by antibodies of the invention include obesity and obesity-related diseases, such as obesity-related orphan diseases. Obesity is a disease caused by excessive food energy intake, lack of physical activity, and/or genetic susceptibility. A body mass index (BMI) >35 indicates severe obesity, a BMI >40 indicates morbid obesity, and a BMI >45 indicates super obesity. Obesity-related diseases include diseases and disorders that are associated with, are caused by, or result from obesity. Examples of obesity-related diseases include cardiovascular diseases, type 2 diabetes, sleep apnea, cancer, osteoarthritis, asthma, fatty liver, and non-alcoholic steatohepatitis (NASH).

NASH is characterized by hepatic inflammation and fat accumulation. The primary risk factors are obesity, diabetes, and dyslipidemia. There is a strong link with cirrhosis and hepatocarcinoma. NASH is associated with elevated AST/ALT (ratio of concentration of aspartate transaminase (AST) and alanine transaminase (ALT)), often without symptoms. Treatments for NASH include lifestyle changes (diet and exercise), bariatric surgery, and pharmaceuticals with mechanisms including absorption reduction (Xenical/Alli (lipase inhibitor)), appetite suppression (Belviq, Byetta, Symlin, Qsymia), and metabolic stimulation (Beloranib).

Examples of obesity-related orphan diseases include Prader-Willi syndrome (e.g., with hyperphagia), craniopharyngioma (e.g., with hyperphagia), Bardet-Biedl syndrome, Cohen syndrome, and MOMO syndrome. Prader-Willi syndrome is a rare genetic disease caused by gene deletion/silencing on chromosome 15. The symptoms include neurocognitive symptoms (intellectual disability, autistic behaviors, uncontrolled appetite (hypothalamic)), slow metabolism, and endocrine disorders (e.g., growth hormone deficiency (GHD), adrenal deficiency (AD)).

Antibodies are administered in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an antibody are 0.1-20, or 0.5-5 mg/kg body weight (e.g., 0.5, 1, 2, 3, 4 or 5 mg/kg) or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Some antibodies can be administered into the systemic circulation by intravenous or subcutaneous administration. Intravenous administration can be, for example, by infusion over a period such as 30-90 min.

The frequency of administration depends on the half-life of the antibody in the circulation, the condition of the patient and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

Treatment with antibodies described herein can be combined with other treatments effective against the disorder being treated. For treatment of immune disorders, conventional treatments include mast cell degranulation inhibitors, corticosteroids, nonsteroidal anti-inflammatory drugs, and stronger anti-inflammatory drugs such as azathioprine, cyclophosphamide, leukeran, FK506 and cyclosporine. Biologic anti-inflammatory agents including or Humira® (adalimumab) can also be used. When used in treating cancer, the antibodies can be combined with chemotherapy, radiation, stem cell treatment, surgery, or treatment with other biologics including Herceptin® (trastuzumab) against the HER2 antigen, Avastin® (bevacizumab) against VEGF, or antibodies to the EGF receptor, such as (Erbitux®, cetuximab), and Vectibix® (panitumumab). Chemotherapy agents include chlorambucil, cyclophosphamide or melphalan, carboplatinum, daunorubicin, doxorubicin, idarubicin, and mitoxantrone, methotrexate, fludarabine, and cytarabine, etoposide or topotecan, vincristine and vinblastine.

VI. Other Applications

The antibodies can be used for detecting laminin α4 in the context of research. The antibodies can also be used for detecting the LG1-3 modules of the G domain of laminin α4, or fragments thereof, in the context of research. The antibodies can also be used as research reagents for laboratory research in detecting laminin α4, or more specifically, the LG1-3 modules of the G domain, or fragments thereof, of laminin α4. In such uses, antibodies can be labeled with fluorescent molecules, spin-labeled molecules, enzymes, or radioisotopes, and can be provided in the form of kit with all the necessary reagents to perform the assay for laminin α4, or more specifically, the LG1-3 modules of the G domain of laminin α4, or fragments thereof. The antibodies can also be used to purify laminin α4, laminins containing laminin α4, or binding partners of laminin α4, e.g., by affinity chromatography.

The antibodies can also be used for inhibiting binding of laminin α4 to MCAM in a biological sample. Inhibition may be demonstrated in a binding assay in which the antibodies of the invention are pre-incubated with recombinant laminin α4 protein, laminin-α4-positive tissue, or laminin-α4-displaying cells, after which recombinant MCAM or MCAM-expressing cells are then assessed for their ability to bind to laminin α4. Exemplary assay formats for showing inhibition are provided in the examples. Optionally, inhibition of a test antibody can be demonstrated in comparison to an irrelevant control antibody not binding to the LG1-3 modules of the G domain of laminin α4 or in comparison to vehicle lacking any antibody. In some instances, binding of laminin α4 to MCAM is inhibited by at least 10%, 20%, 25%, 30%, 40%, 50%, or 75%, (e.g., 10%-75% or 30%-70%).

The antibodies can also be used for inhibiting binding of laminin α4 to integrin α6β1 in a biological sample. Inhibition may be demonstrated in a binding assay assessing the ability of integrin-α6β1-expressing cells to bind laminin α4 in the presence or absence of the antibodies of the invention. Exemplary assay formats for showing inhibition are provided in the examples. Optionally, inhibition of a test antibody can be demonstrated in comparison to an irrelevant control antibody not binding to the LG1-3 modules of the G domain of laminin α4 or in comparison to vehicle lacking any antibody. In some instances, binding of laminin α4 to integrin α6β1 is inhibited by at least 10%, 20%, 25%, 30%, 40%, 50%, or 75%, (e.g., 10%-75% or 30%-70%).

The antibodies can also be used for inhibiting cell adhesion in a biological sample. Preferably, the cell adhesion is dependent on laminin α4. For example, the cell adhesion is mediated by the LG1-3 modules of the G domain of laminin α4. An exemplary cell adhesion assay is described in the examples. In some instances, cell adhesion is inhibited by at least 10%, 20%, 25%, 30%, 40%, 50%, or 75%, (e.g., 10%-75% or 30%-70%).

The antibodies can also be used for inhibiting laminin-α4-induced pAkt activation in a biological sample. An exemplary assay is described in the examples. In some methods, laminin-α4-induced pAkt activation is inhibited by at least 10%, 20%, 25%, 30%, 40%, 50%, or 75%, (e.g., 10%-75% or 30%-70%).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1

### Circulating Recombinant MCAM Extracellular Domain and Anti-LAMA4 Antibodies Specifically Localize to Choroid Plexus, a Major T-Cell Entry Point in CNS

The function of MCAM has been discussed in tumor and autoimmunity models, with MCAM expression reported to confer an adhesive and infiltrative phenotype to tumor and TH17 cells. Furthermore, the α4 chain of Laminin 411 has been reported to be a ligand of MCAM. Consequently, through the use of both LAMA4-/- mice and anti-MCAM monoclonal antibodies targeting MCAM-LAMA4 binding, LAMA4 has been reported to be important for mediating MCAM-LAMA4 adhesion for T-cell infiltration and associated CNS inflammatory symptoms in a mouse model of autoimmunity. See, e.g., Xie et al., Cancer Res. 57: 2295-2303 (1997), Flanagan et al., PLoS ONE. 7(7): e40443 (2012), and Wu et al., Nat Med. 15: 519-527. (2009). Although these reports indicate that MCAM-LAMA4 interactions are important for TH17 cell infiltration of the CNS, it was unknown whether targeting the MCAM-LAMA4 binding interaction using monoclonal anti-LAMA4 antibodies would be efficacious in blocking MCAM-LAMA4 binding and subsequent T cell infiltration of the CNS. It was thus of great interest to determine (1) where the primary CNS entry point(s) for MCAM-expressing T-cells is located in the uninflamed brain and (2) whether a circulating anti-LAMA4 antibody could access this primary CNS entry point(s).

To identify the primary CNS entry point for MCAM+ T-cells, an MCAM-Fc fusion protein was generated and intravenously injected into healthy mice. Pan-laminin and MCAM staining of the choroid vasculature in the CNS was undertaken. The staining showed that MCAM-Fc specifically localizes to the choroid plexus vasculature in the CNS while Fc control protein does not. LAMA4 and pan-laminin staining of choroid tissue, and LAMA4 and MCAM staining of choroid tissue were also undertaken. The staining showed that LAMA4 and MCAM colocalize at the choroidal endothelial basement membrane but not the pan-laminin-positive basement membrane. These results suggested that LAMA4 may mediate both endothelial-basement membrane adhesion with MCAM and vascular adhesion/migration of circulating MCAM-expressing T-cells.

Because MCAM-Fc appears to specifically accumulate at the basement membrane surrounding the choroidal endothelium, we hypothesized that MCAM-Fc localization is driven by circulation-accessible LAMA4 protein. Pan-laminin and LAMA4 staining of the choroid vasculature in the CNS was undertaken. The staining showed that intravenously administered anti-LAMA4 antibody (compared to isotype control antibody) specifically localized to the choroid plexus vasculature/basement membrane network in an identical fashion to MCAM-Fc. These results are consistent with a model whereby TH17 cells enter into the brain via the choroid plexus through a MCAM-LAMA4-driven mechanism. To provide further support for this model, LAMA4 and CD4 staining of choroid tissue was undertaken. This staining detected CD4+ T-cells crossing the LAMA4+ choroid basement membrane and into the stromal space in an inflamed mouse brain.

### Example 2

### Anti-LAMA4 Antibodies Block MCAM-LAMA4 Binding

Monoclonal antibodies against LAMA4 were generated as described in the Materials and Methods. The specific binding between the monoclonal antibodies and LAMA4 was confirmed by assessing the monoclonal antibodies' ability to stain wild-type tissue versus LAMA4-/- mouse tissue. Antibody 5Al2, directly conjugated to 650, showed specific staining of LAMA4-positive mouse tissue while failing to stain LAMA4-/- tissue above background levels.

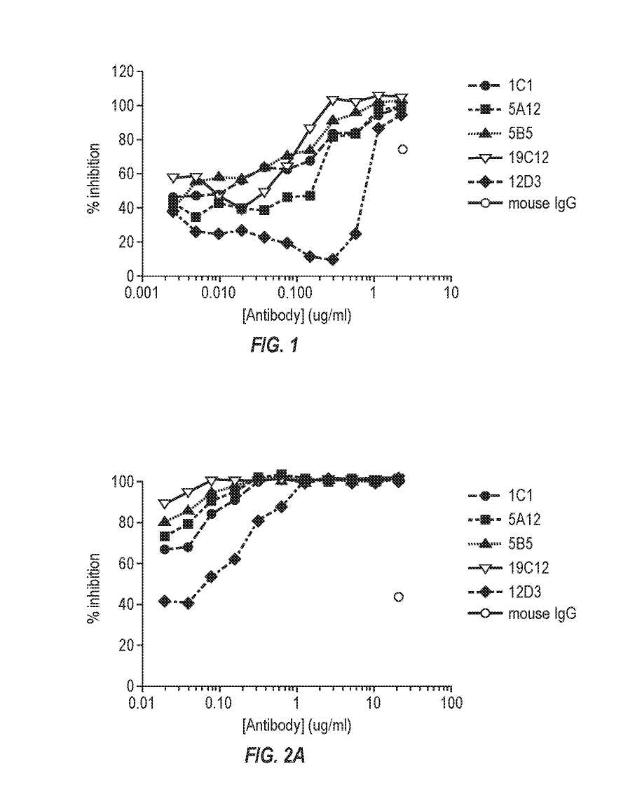

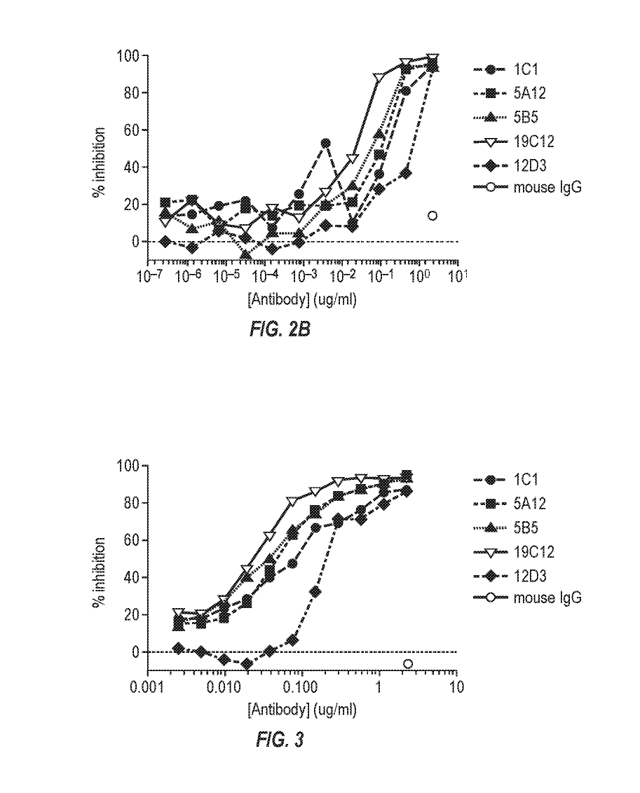

The monoclonal antibodies against LAMA4 were tested for their ability to block the binding of LAMA4 to its ligand MCAM. IgG control antibody, 1C1, 5Al2, 5B5, 19C12, and 12D3 were pre-incubated with recombinant LAMA4 protein, LAMA4-positive healthy mouse brain tissue, or LAMA4-displaying human 293 cells. Recombinant MCAM-Fc or MCAM-expressing CHO cells were then assessed for their ability to bind to LAMA4 as demonstrated by ELISA (FIG. 1), LAMA4 pDisplay flow cytometric blocking assay (FIG. 2A and B, showing higher and lower antibody concentrations, respectively), hMCAM.CHO flow cytometric blocking assay (FIG. 3), and mouse brain tissue blocking assay, as described in the Materials in Methods. For the mouse brain tissue blocking assay, antibodies were used at concentrations of 2.5 ug/ml or 0.04 ug/ml. These assays all showed that 1C1, 5Al2, 5B5, 19C12, and 12D3 can block binding of MCAM and LAMA4.

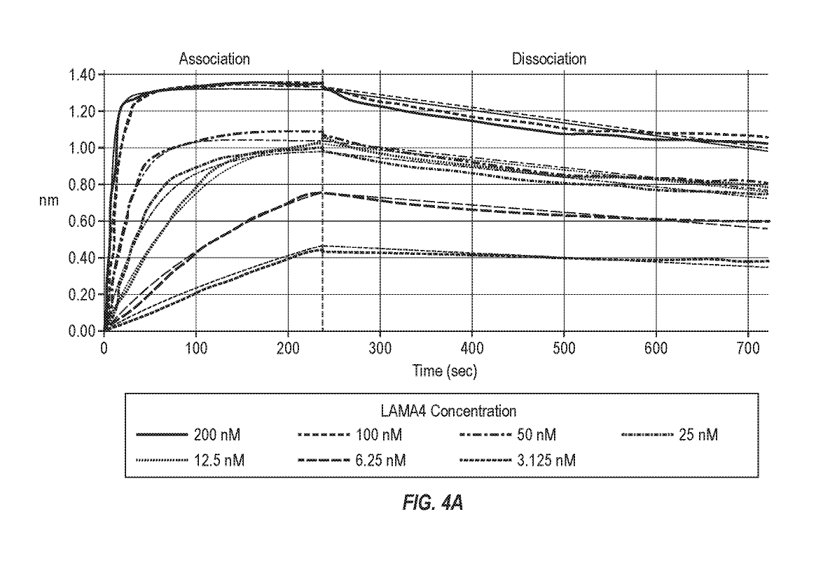

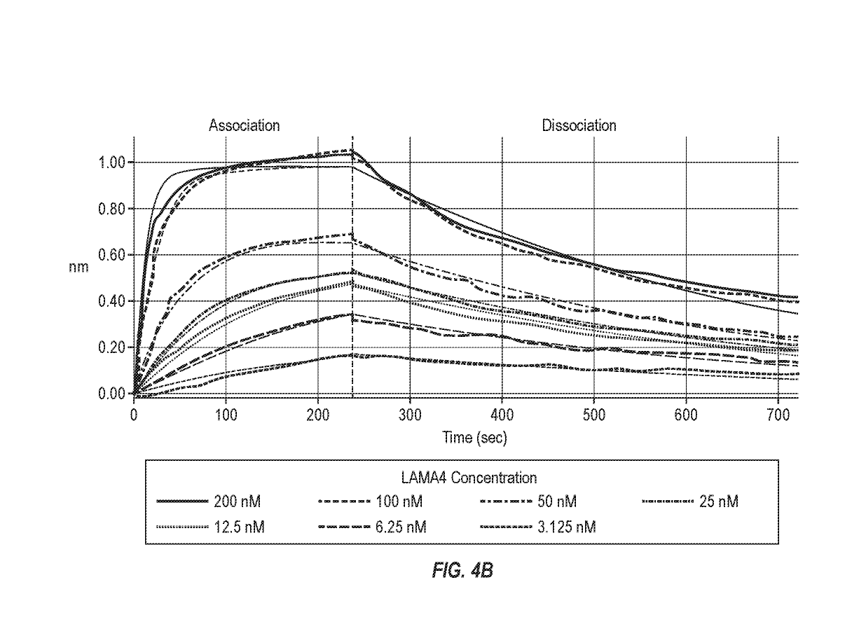

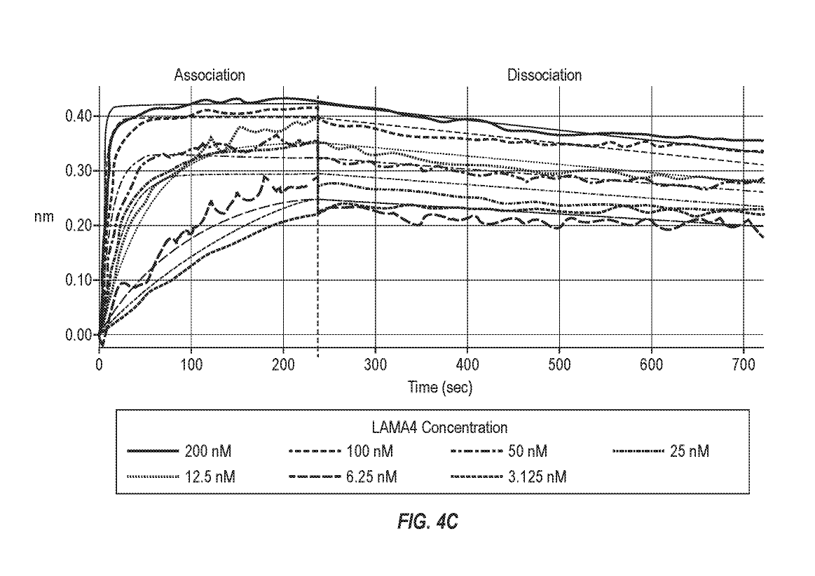

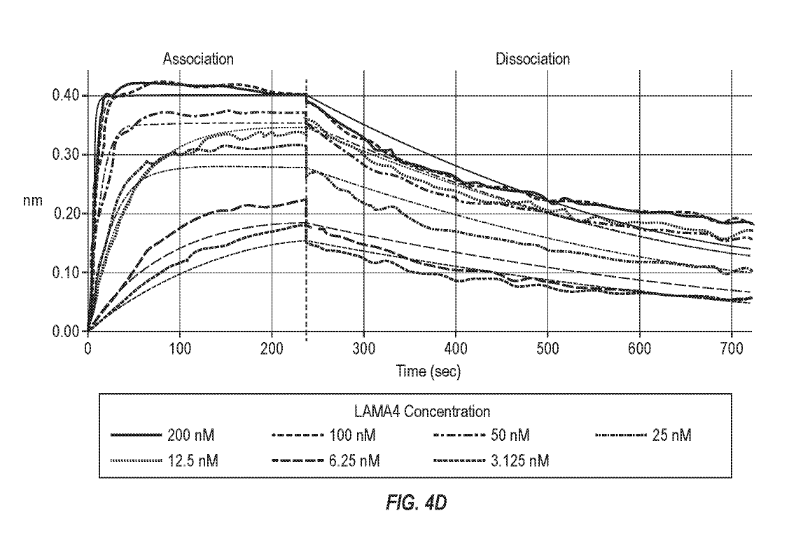

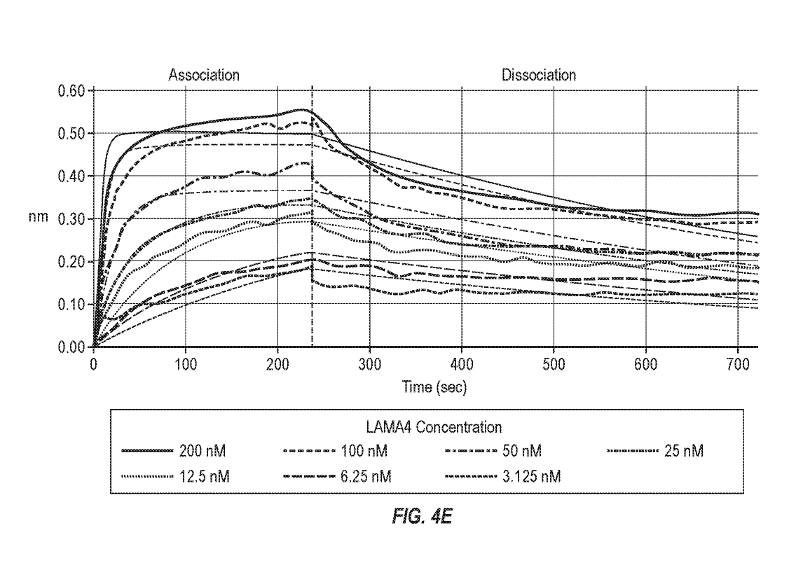

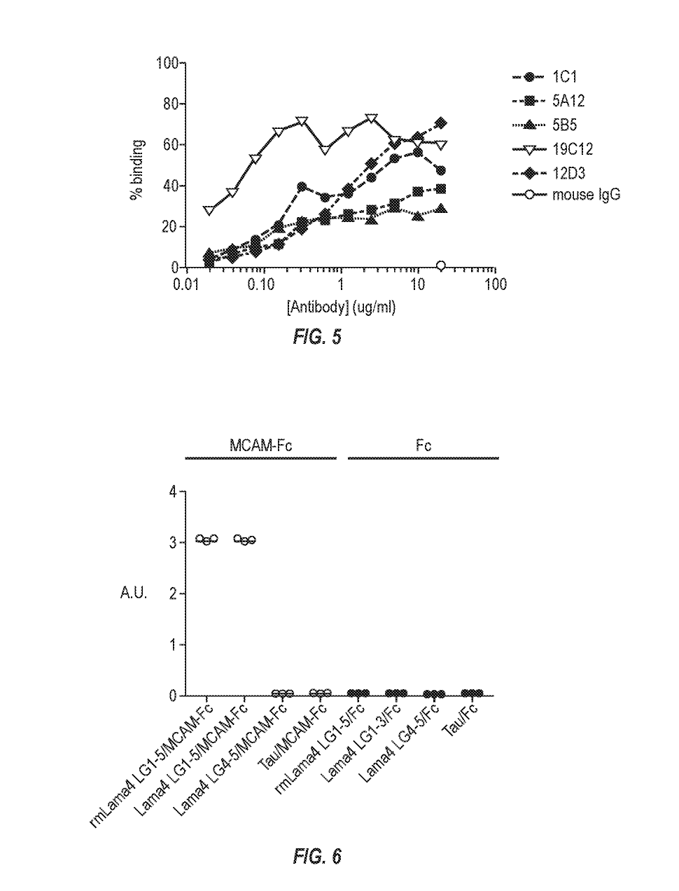

To compare antibody blocking with LAMA4 antibody binding activity, relative binding and on/off rates were analyzed by ForteBio and Biacore as shown in FIG. 4 and Table 1, respectively. ForteBio analysis for the 19C12, 1C1, 5Al2, 5B5, and 12D3 antibodies is shown in FIG. 4A-E, respectively. Antibody concentrations were kept constant at 100 nM, and the concentration of LAMA4 was varied as indicated in FIG. 4A-E. For each concentration of LAMA4, two lines are presented in FIG. 4A-E: a bolded line representing the raw data and a non-bolded line representing the statistical fitting of the raw data. Both ForteBio and Biacore analysis demonstrate that antibody binding activity correlates with blocking activity: 19C12 was the strongest binder while 12D3 was the weakest. To verify these results, binding of IgG control antibody, 19C12, 1C1, 5Al2, 5B5, and 12D3 to LAMA4-displaying human 293 cells was tested as shown in FIG. 5. 19C12 was again shown to be the best binding antibody.

TABLE 1
Biacore Assay Comparing Binding of 12D3, 5B5, 19C12, 1C1, and 5A12 to LAMA4

| | Human LAMA4 | | | Murine LAMA4 | | |
|---|---|---|---|---|---|---|
| Antibody | kₐ (M⁻¹s⁻¹) | k_{d} (s⁻¹) | K_{D} (M) | kₐ (M⁻¹s⁻¹) | k_{d} (s⁻¹) | K_{D} (M) |
| 12D3 | 3.66 × 10⁵ | 1.09 × 10⁻² | 2.97 × 10⁻⁸ | 4.26 × 10⁵ | 1.15 × 10⁻² | 2.71 × 10⁻⁸ |
| 5B5 | 1.43 × 10⁶ | 1.05 × 10⁻² | 7.33 × 10⁻⁹ | 2.20 × 10⁶ | 1.16 × 10⁻² | 5.27 × 10⁻⁹ |
| 19C12 | 6.72 × 10⁶ | 8.58 × 10⁻³ | 1.27 × 10⁻⁹ | 6.32 × 10⁶ | 6.10 × 10⁻³ | 9.65 × 10⁻¹⁰ |
| 1C1 | 6.10 × 10⁵ | 9.17 × 10⁻³ | 1.33 × 10⁻⁸ | 5.90 × 10⁵ | 9.47 × 10⁻³ | 1.61 × 10⁻⁸ |
| 5A12 | 7.23 × 10⁵ | 7.91 × 10⁻³ | 1.09 × 10⁻⁸ | 7.95 × 10⁵ | 8.15 × 10⁻³ | 1.03 × 10⁻⁸ |

These data indicate that clones 1C1, 5Al2, 5B5, 19C12, and 12D3 are all capable of specifically blocking the binding of human MCAM to its ligand LAMA4 and can be useful for treating multiple sclerosis by inhibiting MCAM-mediated adhesion of TH17 cells to the vasculature and blocking the migration of TH17 cells into central nervous system.

### Example 3

### Epitope Determination of MCAM Binding and Blocking Anti-LAMA4 Monoclonal Antibodies

To determine the LAMA4 epitope(s)/domain(s) necessary for MCAM binding, recombinant MCAM-Fc (or Fc control) protein was prebound to plates via goat anti-human Fabs overnight. Truncated recombinant variants of the LAMA4 G domain (and Tau control protein) were assayed for their ability to bind MCAM-Fc protein as described in the Materials and Methods and as shown in FIG. 6. The results are presented in arbitrary units (A.U.) on the y-axis. Whereas Fc control protein failed to bind any LAMA4 variants, LAMA4 variants containing LG modules 1-5 and 1-3 were able to robustly bind MCAM-Fc protein. A LAMA4 variant containing the LG modules 4-5 failed to bind MCAM-Fc protein, as did Tau. Therefore, the LG1-3 modules of the G domain of LAMA4 mediate LAMA4-MCAM interactions.

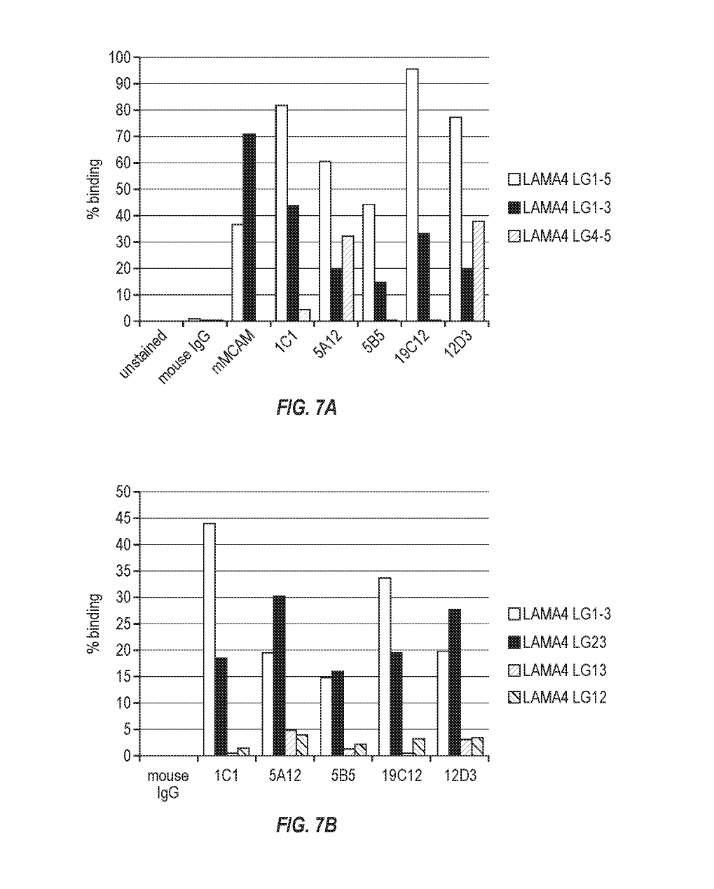

To verify these ELISA-based results, binding of LAMA4-displaying human embryonic kidney cells (293) to recombinant 650-labeled MCAM-Fc was assessed by flow cytometry as shown in FIG. 7A and B. FIG. 7A shows binding of 293 cells displaying LAMA4 variants with LG1-5, LG1-3, and LG4-5. FIG. 7B shows binding of 293 cells displaying LAMA4 variants with LG1-3, LGde 1 (LG23), LGde2 (LG13), and LGde3 (LG23). LGde1 has a full-length G domain (i.e., LG1-5) with LG1 deleted, LGde2 has a full-length G domain with LG2 deleted, and LGde3 has a full-length G domain with LG3 deleted. Recombinant MCAM-Fc protein was able to specifically bind 293 cells expressing LAMA4 variants LG1-5, LG1-3, and LGde 1 (LG23), but not 293 cells expressing LAMA 4 variants LG4-5, LGde2 (LG13), or LGde3 (LG12). These results indicate that the anti-LAMA4 monoclonal antibodies that block MCAM-LAMA4 binding can bind within the LG2 and LG3 modules of the G domain. In addition, 1C1, 5Al2, 5B5, 19C12, and 12D3 bind in a similar fashion as MCAM-Fc protein, demonstrating that the LG1-3 modules of the G domain of LAMA4 mediate both MCAM-Fc protein binding and binding of these anti-LAMA4 blocking antibodies.

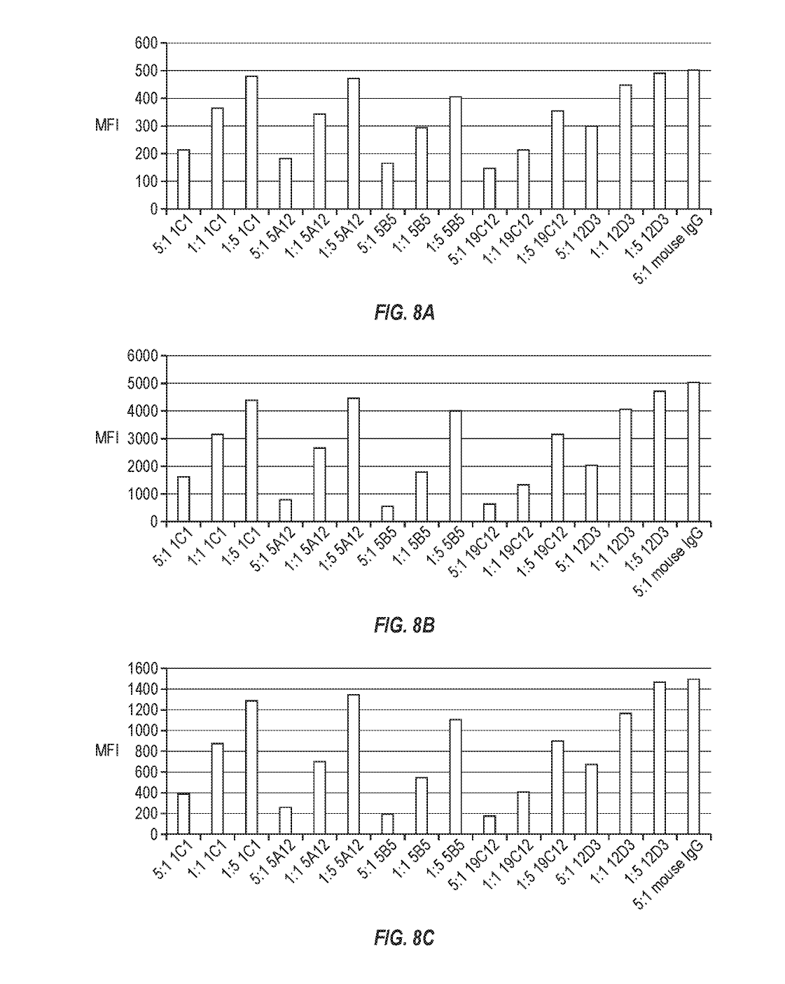

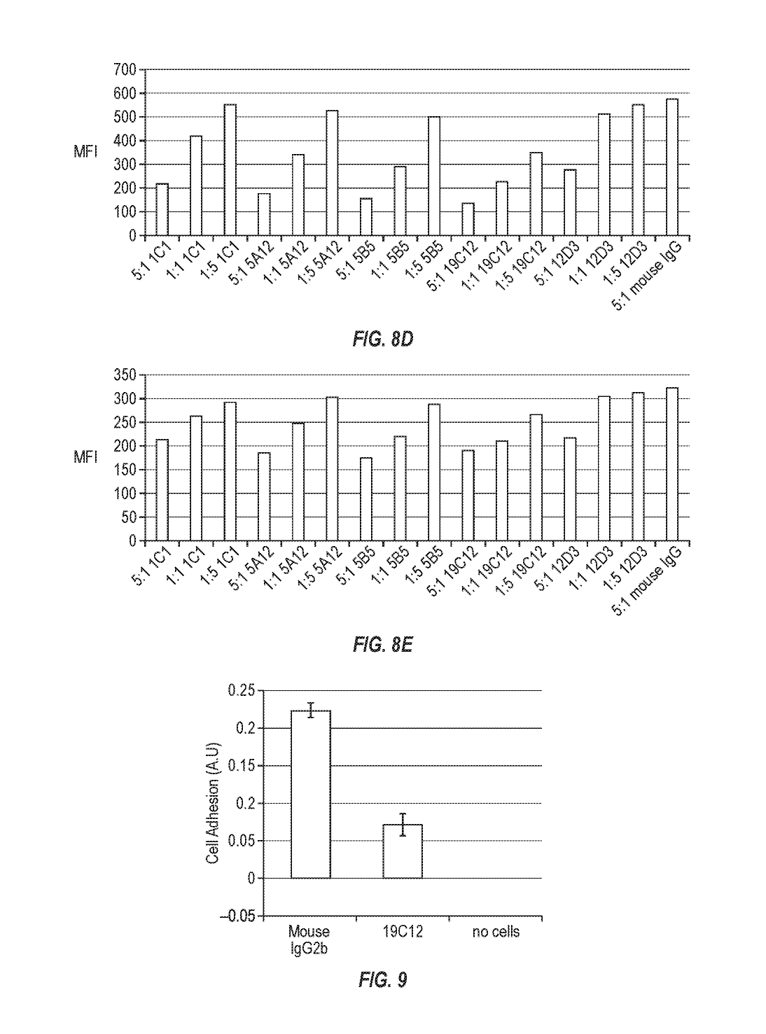

Competition experiments were carried out to differentiate the 5Al2, 19C12, 1C1, 5B5, and 12D3 antibodies by epitope binding. Binding of the antibodies to LAMA4-displaying human embryonic kidney cells (293) was assessed using decreasing ratios (5:1, 1:1, and 1:5) of blocking antibody to 650-labeled binding antibody, with mouse IgG1 used as a negative control. Binding of the 5Al2, 19C12, 1C1, 5B5, and 12D3 antibodies was assessed by flow cytometry as shown in FIG. 8 A-E, respectively. All five blocking antibodies are able to compete with each other for LAMA4 binding, with each being having higher blocking efficacy at the 5:1 ratio ((blocking antibody):(binding antibody)) and lower blocking efficacies as the ratio decreases. These results indicate that the anti-LAMA4 antibodies all bind similar epitopes on the LAMA4 protein.

### Example 4

### Anti-LAMA4 Antibody 19C12 Blocks Integrin 41601 Binding and Human Melanoma Cell Adhesion

To determine the functional consequences of targeting LAMA4-MCAM binding via anti-LG1-3 antibodies, recombinant LAMA4-coated ELISA plates were incubated with 20 ug/ml 19C12 (or mouse IgG2b control) and were then assayed for their ability to bind human melanoma cell line WM-266-4 as described in the Materials and Methods and as shown in FIG. 9. The results are presented in arbitrary units (A.U.) on the y-axis. Whereas mouse IgG2b control failed to block LAMA4-mediated cell adhesion, 19C12 was able to inhibit LAMA4-mediated human melanoma cell adhesion by approximately 80%. These results indicate that anti-LG1-3 antibodies can block cell adhesion events necessary for tumor cell adhesion, proliferation, and metastasis.

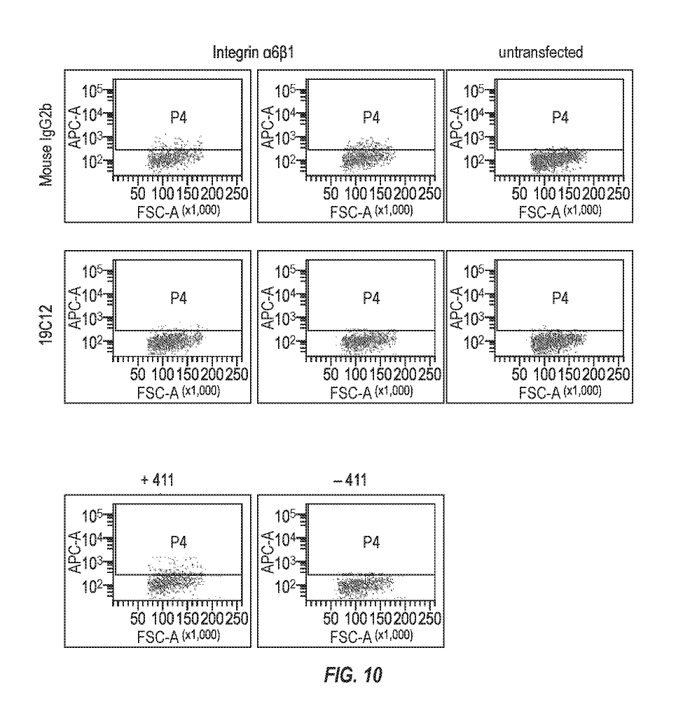

To test the hypothesis that 19C12 can block LAMA4-mediate cell adhesion via both MCAM and integrin interference, LAMA4 binding (in complex with its gamma1 and beta1 chains as laminin 411) was assessed via flow cytometry analysis using integrin-α6β1-expressing human 293 cells, as shown in FIG. 10. LAMA4 interacts with integrin-overexpressing cells, and 19C12 was able to completely block LAMA4 binding to integrin-α6β1-expressing 293 human cells whereas mouse IgG2b control was not able to do so, as shown by comparison of the P4 areas.

In another experiment, adherent 293T cells were transiently transfected in 6-well plates with 3 ug integrin beta1 and 1 ug integrin alpha 6 plasmid. 1 mM MnCl₂+ was used to activate the integrins. The transiently transfected adherent 293T cells expressing human integrin α6β1 were shown via flow cytometry to bind to laminin 411 (alpha 4, beta1, gamma1). Anti-LAMA4-650 antibody was used to detect bound laminin 411. Binding was inhibited by MCAM-Fc, 5 mM EDTA, or 19C12. Fc alone, buffer, and mouse IgG2b isotype control served as controls and failed to inhibit binding. These data indicate that MCAM and integrin α6β1 recognize a similar region of LAMA4.

These data show that anti-LG1-3 antibodies block WM-266-4 human melanoma cell adhesion via inhibiting LAMA4 interactions with both MCAM and integrin molecules and indicate that targeting LG1-3 can be efficacious in slowing tumor growth and metastasis.

### Example 5

### Design of Humanized 19C12 Antibodies

The starting point or donor antibody for humanization is the mouse antibody 19C12. The heavy chain variable amino acid sequence of mature m19C12 is provided as SEQ ID NO:15. The light chain variable amino acid sequence of mature m19C12 is provided as SEQ ID NO:16. The heavy chain CDR1, CDR2, and CDR3 amino acid sequences are provided as SEQ ID NOS:19, 20, and 21, respectively (as defined by Kabat). The light chain CDR1, CDR2, and CDR3 amino acid sequences are provided as SEQ ID NOS:22, 23, and 24, respectively (as defined by Kabat). Kabat numbering is used throughout in this Example.

The variable kappa (Vk) of m19C12 belongs to mouse Kabat subgroup 5, which corresponds to human Kabat subgroup 1. The variable heavy (Vh) of m19C12 belongs to mouse Kabat subgroup 5a, which corresponds to human Kabat subgroup 1. See Kabat et al. Sequences of Proteins of Immunological Interest, Fifth Edition. NIH Publication No. 91-3242, 1991. The 17-residue CDR-L1 belongs to canonical class 3, the 7-residue CDR-L2 belongs to canonical class 1, and the 9-residue CDR-L3 belongs to canonical class 1 in Vk. See Martin & Thornton, J. Mol. Biol. 263:800-15, 1996. The 5-residue CDR-H1 belongs to canonical class 1, and the 17-residue CDR-H2 belongs to canonical class 2. See Martin & Thornton, J Mol. Biol. 263:800-15, 1996. The CDR-H3 has no canonical classes, but the 6-residue loop probably has a kinked base according to the rules of Shirai et al., FEBS Lett. 455:188-97 (1999).

The residues at the interface between the Vk and Vh domains are usual.

A search was made over the protein sequences in the PDB database (Deshpande et al., Nucleic Acids Res. 33: D233-7, 2005) to find structures which would provide a rough structural model of 19C12. The crystal structure of the antibody against dengue virus serotypes 1, 2, and 3 was used for Vk structure. It retains the same canonical structure for the loops as 19C12 (pdb code 2R29, resolution 3.0A). The heavy chain of the antibody against human rhinovirus 14 (HRV14) (pdb code 1FOR, resolution 2.7A) was used for Vh structure. It contains the same canonical structures for CDR-H1 and CDR-H2 as that of 19C12VH, and also the same length CDR-H3 with a kinked base. BioLuminate was used to model a rough structure of 19C12Fv.

A search of the non-redundant protein sequence database from NCBI with CDR“X”ed 19C12Fv allowed selection of suitable human frameworks into which to graft the murine CDRs. For Vh, one human Ig heavy chain, having NCBI accession code BAC01530.1 (SEQ ID NO:75), was chosen. It shares the canonical form of 19C12 CDR-H1 and H2, and H3 is 10 residues long with a predicted kinked base. For Vk, two human kappa light chains, having NCBI accession codes ABA71367.1 (SEQ ID NO:76) and ABI74162.1 (SEQ ID NO:77), were chosen. They have the same canonical classes for CDR-L1, L2 and L3 as that for the parental Vk. Humanized 19C12 heavy and light chain variable region sequences having no backmutations or other mutations are provided as SEQ ID NOS:78 and 79.

Three humanized heavy chain variable region variants and six humanized light chain variable region variants were constructed containing different permutations of substitutions (Hu19C12VHv1-3 (SEQ ID NOS:80-82) and Hu19C12VLv1-6 (SEQ ID NOS:83-88)) (Tables 2-5). The exemplary humanized Vh and Vk designs, with backmutations and other mutations based on selected human frameworks, are shown in Tables 2 and 3, respectively. The gray-shaded areas in the first column in Tables 2 and 3 indicate the CDRs as defined by Chothia, and the gray-shaded areas in the remaining columns in Tables 2 and 3 indicate the CDRs as defined by Kabat. SEQ ID NOS:80-88 contain backmutations and other mutations as shown in Table 4. The amino acids at positions H1, H11, H12, H16, H20, H27, H28, H38, H43, H48, H69, H91, H108, L1, L9, L22, L49, L68, L76, L77, L78, L79, L85, and L100 in Hu19C12VHv1-3 and Hu19C12VLv1-6 are listed in Table 5.

TABLE 4
V_{H}, V_{L} Backmutations and Other Mutations

| | | Donor Framework |
|---|---|---|
| V_{L} Variant | V_{L} Exon Acceptor Sequence | Residues |
| Hu19C12VLv1 | NCBI accession codes | L1, L9, L22, L49, |
| (SEQ ID NO: 83) | ABA71367.1 and ABI74162.1 | L68, L85 |
| | (SEQ ID NOS: 76 and 77) | |
| Hu19C12VLv2 | NCBI accession codes | L1, L9, L22, L85 |
| (SEQ ID NO: 84) | ABA71367.1 and ABI74162.1 | |
| | (SEQ ID NOS: 76 and 77) | |
| Hu19C12VLv3 | NCBI accession codes | L1, L9, L22, L49, |
| (SEQ ID NO: 85) | ABA71367.1 and ABI74162.1 | L68, L76, L77, |
| | (SEQ ID NOS: 76 and 77) | L78, L79, L85, L100 |
| Hu19C12VLv4 | NCBI accession codes | L1, L9, L22, L77, |
| (SEQ ID NO: 86) | ABA71367.1 and ABI74162.1 | L78, L79, |
| | (SEQ ID NOS: 76 and 77) | L85, L100 |
| Hu19C12VLv5 | NCBI accession codes | L9, L22, L77, L85 |
| (SEQ ID NO: 87) | ABA71367.1 and ABI74162.1 | |
| | (SEQ ID NOS: 76 and 77) | |
| Hu19C12VLv6 | NCBI accession codes | L9, L22, L77, L78, |
| (SEQ ID NO: 88) | ABA71367.1 and ABI74162.1 | L79, L85, L100 |
| | (SEQ ID NOS: 76 and 77) | |
| Hu19C12VHv1 | NCBI accession code | H11, H12, H16, H20, |
| (SEQ ID NO: 80) | BAC01530.1 | H27, H28, H38, |
| | (SEQ ID NO: 75) | H43, H48, H69, |
| | | H91, H108 |
| Hu19C12VHv2 | NCBI accession code | H11, H12, H16, |
| (SEQ ID NO: 81) | BAC01530.1 | H27, H28, H48, |
| | (SEQ ID NO: 75) | H91, H108 |
| Hu19C12VHv3 | NCBI accession code | H1, H11, H12, H16, |
| (SEQ ID NO: 82) | BAC01530.1 | H27, H28, H48, |
| | (SEQ ID NO: 75) | H91, H108 |

TABLE 5
Kabat Numbering of Framework Residues
for Backmutations and Other Mutations in Humanized 19C12 Antibodies

| | ABA71367.1 | ABI74162.1 | BAC01530.1 | | | | |
|---|---|---|---|---|---|---|---|
| | light chain | light chain | heavy chain | Mouse 19C12 | Hu19C12VL1 | Hu19C12VL2 | Hu19C12VL3 |
| L1 | D | E | — | N | N | N | N |
| L9 | L | D | — | A | A | A | A |
| L22 | N | N | — | S | S | S | S |
| L49 | S | Y | — | C | C | S | C |
| L68 | G | E | — | R | R | G | R |
| L76 | D | S | — | D | S | S | D |
| L77 | N | S | — | P | S | S | P |
| L78 | L | L | — | V | L | L | V |
| L79 | Q | Q | — | E | Q | Q | E |
| L85 | L | V | — | T | T | T | T |
| L100 | Q | Q | — | A | Q | Q | A |
| H1 | — | — | Q | Q | — | — | — |
| H11 | — | — | V | L | — | — | — |
| H12 | — | — | K | V | — | — | — |
| H16 | — | — | S | A | — | — | — |
| H20 | — | — | V | I | — | — | — |
| H27 | — | — | G | Y | — | — | — |
| H28 | — | — | T | A | — | — | — |
| H38 | — | — | R | K | — | — | — |
| H43 | — | — | Q | E | — | — | — |
| H48 | — | — | M | I | — | — | — |
| H69 | — | — | I | L | — | — | — |
| H91 | — | — | Y | F | — | — | — |
| H108 | — | — | M | T | — | — | — |

| | Hu19C12VL4 | Hu19C12VL5 | Hu19C12VL6 | Hu19C12VH1 | Hu19C12VH2 | Hu19C12VH3 |
|---|---|---|---|---|---|---|
| L1 | N | D | D | — | — | — |
| L9 | A | A | A | — | — | — |
| L22 | S | S | S | — | — | — |
| L49 | S | S | S | — | — | — |
| L68 | G | G | G | — | — | — |
| L76 | S | S | S | — | — | — |
| L77 | P | P | P | — | — | — |
| L78 | V | L | V | — | — | — |
| L79 | E | Q | E | — | — | — |
| L85 | T | T | T | — | — | — |
| L100 | A | Q | A | — | — | — |
| H1 | — | — | — | Q | Q | E |
| H11 | — | — | — | L | L | L |
| H12 | — | — | — | V | V | V |
| H16 | — | — | — | A | A | A |
| H20 | — | — | — | I | V | V |
| H27 | — | — | — | Y | Y | Y |
| H28 | — | — | — | A | A | A |
| H38 | — | — | — | K | R | R |
| H43 | — | — | — | E | Q | Q |
| H48 | — | — | — | I | I | I |
| H69 | — | — | — | L | I | I |
| H91 | — | — | — | F | F | F |
| H108 | — | — | — | T | T | T |

The rationales for selection of the above positions in the light chain variable region as candidates for substitution are as follows.

D1N: N contacts LCDR1 and may be critical. D was tried in some other versions because N is rare in the human IgG framework.

L9A: A is more frequent than D in the human framework.

N22S: S contacts F71 in the light chain, which is the canonical residue.

S49C: C may contact LCDR2. S was tried in some other versions.

G68R: R contacts F71 in the light chain, which is the canonical residue. However, G is more frequent than R in the human framework. Thus, R was tried in some versions and G in other versions.

S76D: S is more frequent than D in the human framework. Because D is close to P and may contact P, the critical structure residue, D was tried in some versions.

S77P: Proline cis-trans isomerization plays a key role in the rate-determining steps of protein folding. P was tried in some versions and S in other versions.

L78V: V may contact VL P77 and thus affect folding. L was tried in some other versions.

Q79E: E may contact VL P77 and thus affect folding. Q was tried in some other versions.

L85T: T is more frequent than V in the human framework.

Q100A: A contacts VL Y87, the interface issue, and is therefore critical. Q was tried in some other versions.

The rationales for selection of the above positions in the heavy chain variable region as candidates for substitution are as follows.

Q1E: This is a mutation but not a backmutation. Glutamate (E) conversion to pyroglutamate (pE) occurs more slowly than from glutamine (Q). Because of the loss of a primary amine in the glutamine to pE conversion, antibodies become more acidic. Incomplete conversion produces heterogeneity in the antibody that can be observed as multiple peaks using charge-based analytical methods. Heterogeneity differences may indicate a lack of process control.

V11L: L is more frequent than V in the human IgG framework.

K12V: V is more frequent than K in the human IgG framework.

S16A: A is more frequent than S in the human IgG framework.

V20I: I and V are similarly frequent in the human IgG framework, so I was tried in some versions and V in other versions.

G27Y: This residue is within HCDR1 as defined by Chothia, so Y was used to maintain the binding ability.

T28A: This residue is within HCDR1 as defined by Chothia, so A was used to maintain the binding ability.

R38K: R is more frequent than K in the human IgG framework, but K contacts HQ39 and HW47, the two canonical residues, so K was tried in some versions and R in other versions.

Q43E: Q is more frequent than E in the human IgG framework, but E contacts LY87, the interface residue, so E was tried in some versions and Q in other versions.

M48I: I contacts multiple critical residues including interface residues (HV37 and HW47) and HCDR2 residues and is therefore critical.

I69L: I is more frequent than L in the human IgG framework, but L contacts HCDR2, so I was tried in some versions and L in other versions.

Y91F: F is an interface residue, which is critical.

M108T: T is more frequent than M in the human IgG framework.

The six humanized light chain variable region variants and three humanized heavy chain variable region variants are as follows:

Hu19C12VL version 1 (D1N, L9A, N22S, S49C, G68R, and L85T backmutations in lowercase):

(SEQ ID NO: 83)
nIVLTQSPaSLAVSLGERATIsCRASESVDSYGTSFMHWYQQKPGQPPK
LLIcLASSLESGVPDRFSGSGSrTDFTLTISSLQAEDVAtYYCQQNNED
PPTFGQGTKLEIKR.

Hu19C12VL version 2 (D1N, L9A, N22S, and L85T backmutations in lowercase):

(SEQ ID NO: 84)
nIVLTQSPaSLAVSLGERATIsCRASESVDSYGTSFMHWYQQKPGQPPK
LLISLASSLESGVPDRFSGSGSGTDFTLTISSLQAEDVAtYYCQQNNED
PPTFGQGTKLEIKR.

Hu19C12VL version 3 (D1N, L9A, N22S, S49C, G68R, S76D, S77P, L78V, Q79E, L85T, and Q100A backmutations in lowercase):

(SEQ ID NO: 85)
nIVLTQSPaSLAVSLGERATIsCRASESVDSYGTSFMHWYQQKPGQPPK
LLIcLASSLESGVPARFSGSGSrTDFTLTIdpveAEDAAtYYCQQNNED
PPTFGaGTKLEIKR.

Hu19C12VL version 4 (D1N, L9A, N22S, S77P, L78V, Q79E, L85T, and Q100A backmutations in lowercase):

(SEQ ID NO: 86)
nIVLTQSPaSLAVSLGERATIsCRASESVDSYGTSFMHWYQQKPGQPPK
LLISLASSLESGVPARFSGSGSGTDFTLTISpveAEDAAtYYCQQNNED
PPTFGaGTKLEIKR.

Hu19C12VL version 5 (L9A, N22S, S77P, and L85T backmutations in lowercase):

(SEQ ID NO: 87)
DIVLTQSPaSLAVSLGERATIsCRASESVDSYGTSFMHWYQQKPGQPPK
LLISLASSLESGVPARFSGSGSGTDFTLTISpLQAEDVAtYYCQQNNED
PPTFGQGTKLEIKR.

Hu19C12VL version 6 (L9A, N22S, S77P, L78V, Q79E, L85T, and Q100A backmutations in lowercase):

(SEQ ID NO: 88)
DIVLTQSPaSLAVSLGERATIsCRASESVDSYGTSFMHWYQQKPGQPPK
LLISLASSLESGVPARFSGSGSGTDFTLTISpveAEDAAtYYCQQNNED
PPTFGaGTKLEIKR.

Hu19C12VH version 1 (V11L, K12V, S16A, V20I, G27Y, T28A, R38K, Q43E, M48I, I69L, Y91F, and M108T backmutations in lowercase):

(SEQ ID NO: 80)
QVQLQQSGAElvKPGaSVKISCKASGyaFSTYWMNWVkQAPGeGLEWIG
QIYPGDGDTNYNGKFKGRVT1TADKSTSTAYMELSSLRSEDTAVYfCAR
SDGYYDYWGQGTNTVSS.

Hu19C12VH version 2 (V11L, K12V, S16A, G27Y, T28A, M48I, Y91F, and M108T backmutations in lowercase):

(SEQ ID NO: 81)
QVQLQQSGAElvKPGaSVKVSCKASGyaFSTYWMNWVRQAPGQGLEWIG
QIYPGDGDTNYNGKFKGRVTITADKSTSTAYMELSSLRSEDTAVYfCAR
SDGYYDYWGQGTtVTVSS.

Hu19C12VH version 3 (Q1E mutation and V11L, K12V, S 16A,G27Y, T28A, M48I,

Y91F, and M108T backmutations in lowercase):

(SEQ ID NO: 82)
eVQLQQSGAElvKPGaSVKVSCKASGyaFSTYWMNWVRQAPGQGLEWIG
QIYPGDGDTNYNGKFKGRVTITADKSTSTAYMELSSLRSEDTAVYfCAR
SDGYYDYWGQGTtVTVSS.

### Example 6

### Binding Kinetic Analysis of Humanized 19C12 Antibodies

Binding functions and binding kinetics of humanized 19C12 antibodies comprising a heavy chain selected from Hu19C12VHv1-3 (H1-H3) and/or a light chain selected from Hu19C12VLv1-6 (L1-L6) were characterized.

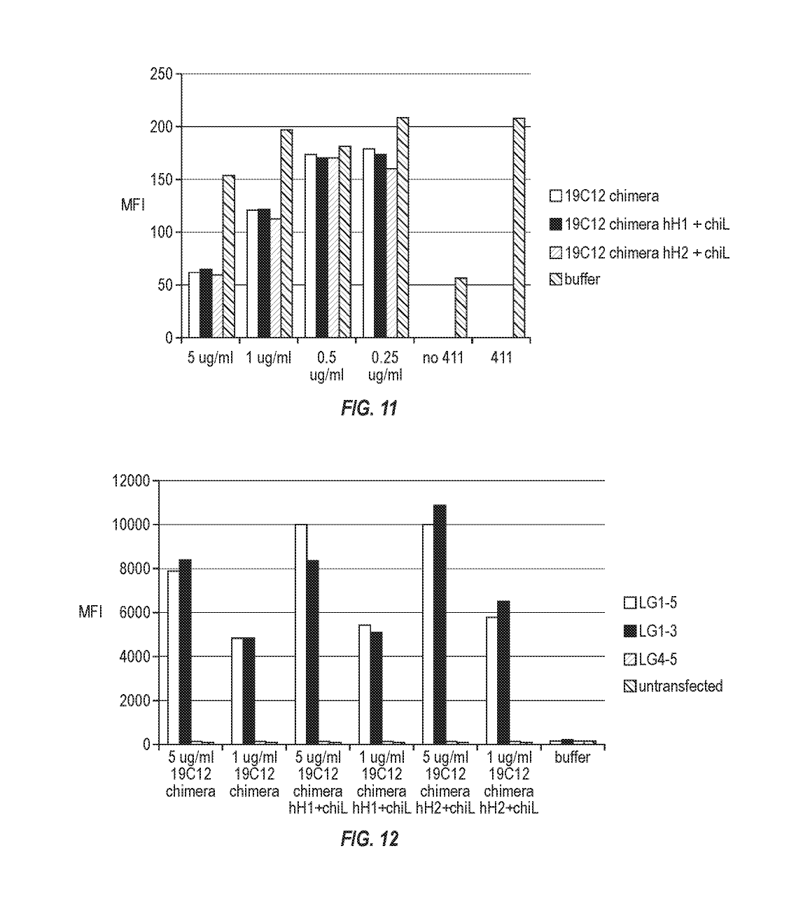

The 19C12 variants chimeric 19C12, H1+chiL, and H2+chiL, and buffer alone were tested for their ability to block the binding of LAMA4 to its ligand MCAM (as shown in FIG. 11) and to bind LAMA4-displaying cells (full length, LG1-3, LG4-5, or untransfected control) (as shown in FIG. 12). To test MCAM/LAMA4 blocking activity, antibodies were pre-incubated with 650-labeled laminin 411 (recombinant laminin trimer containing LAMA4), and then MCAM-expressing CHO cells were assessed for their ability to bind to 650-labeled laminin 411 as described in the Materials and Methods. “No 411” and “411” control conditions marked 100% and 0% blocking activity, respectively. To test LAMA4 binding capacity, serially diluted antibodies were pre-incubated with LAMA4-displaying human 293 cells, followed by anti-human-650 secondary antibody incubation. Fluorescent signal was assessed via flow cytometric analyses and plotted as mean fluorescence intensity (MFI). The blocking and binding experiments demonstrate that all of the 19C12 variants retained their LAMA4 G-domain specificity for LG1-3 and their ability to block binding of MCAM to LAMA4.

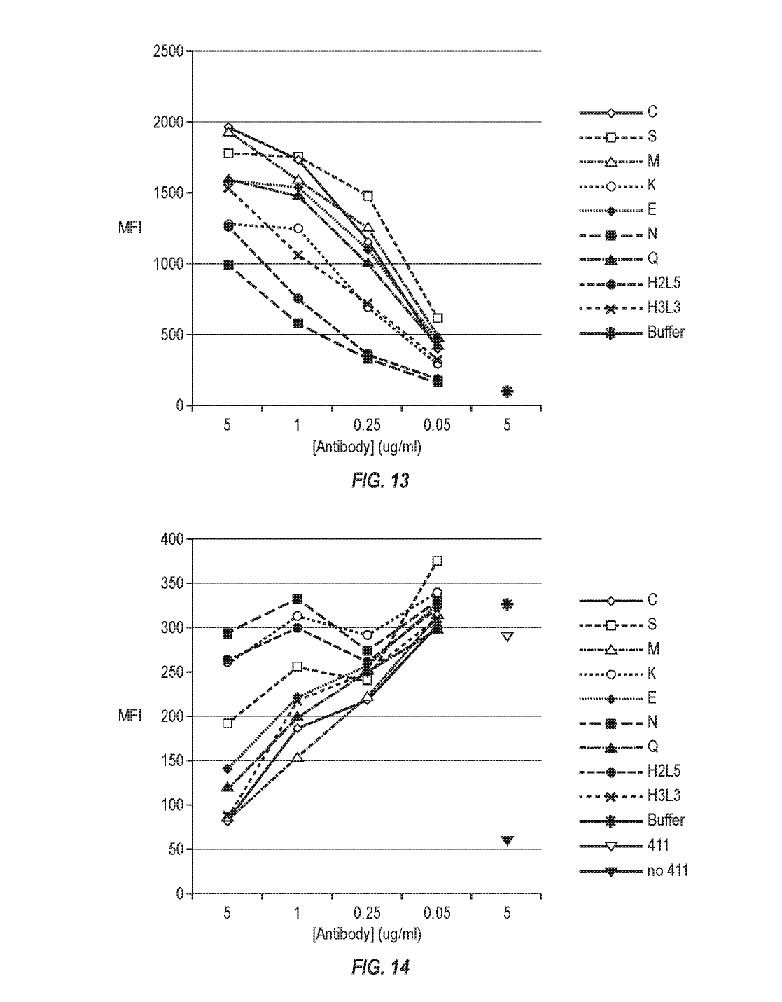

Position L49 of the 19C12 variable light chain was substituted with other amino acids, such as I, T, A, M, Q, or E (see Table 6) because they may confer improved stability relative to substitution to a cysteine. The 19C12 cysteine replacement variants were tested for their ability to bind LAMA4-displaying cells (FIG. 13) and to block the binding of LAMA4 to its ligand MCAM (FIG. 14). To test LAMA4 binding capacity, serially diluted antibodies were pre-incubated with LAMA4-displaying human 293 cells, followed by anti-human-650 secondary antibody incubation. To test MCAM/LAMA4 blocking activity, antibodies were pre-incubated with 650-labeled Laminin 411 (recombinant Laminin trimer containing LAMA4), and then MCAM-expressing CHO cells were assessed for their ability to bind to 650-labeled laminin 411 as described in the Materials and Methods. “No 411” and “411” control conditions marked 100% and 0% blocking activity, respectively. Fluorescent signal was assessed via flow cytometric analyses and plotted as mean fluorescence intensity (MFI). The 19C12 cysteine replacement variants retain their MCAM blocking and LAMA4 binding activity to varying degrees.

TABLE 6
Effect of Substitutions at L49 on Binding to LAMA4

| Amino Acid | Binding to Antigen |
|---|---|
| C | 100% |
| S | 90% |
| D | 60% |
| I | 90% |
| T | 90% |
| G | 50% |
| A | 90% |
| M | 98% |
| K | 50% |
| N | 30% |
| Q | 75% |
| E | 75% |
| Buffer Control | 0% |

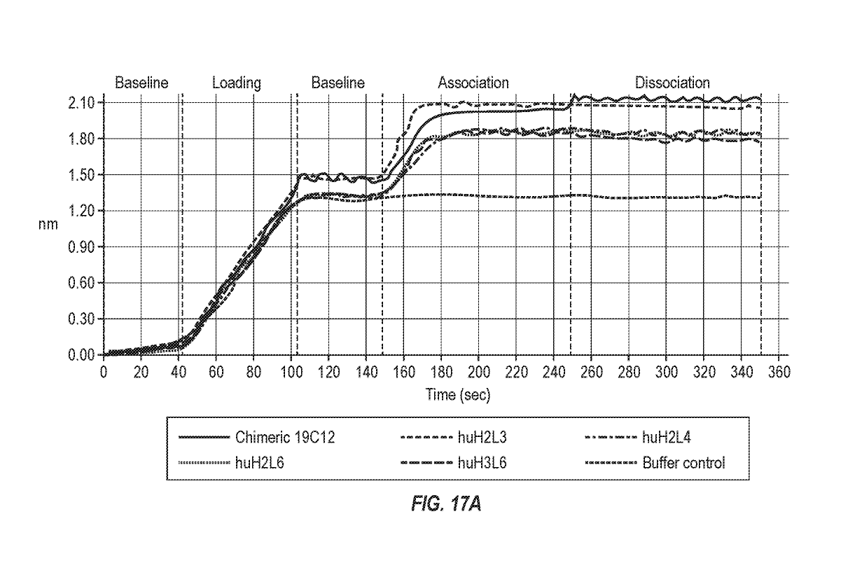

The 19C12 variants chimeric 19C12, H2L3, H2L4, H2L6, H3L6, and isotype control were tested for their ability to block the binding of LAMA4 to its ligand MCAM (FIG. 15) and to bind LAMA4-displaying cells (FIG. 16). To test LAMA4 binding capacity, serially diluted antibodies were pre-incubated with LAMA4-displaying human 293 cells, followed by anti-human-650 secondary antibody incubation. To test MCAM/LAMA4 blocking activity, antibodies were pre-incubated with 650-labeled laminin 411 (recombinant laminin trimer containing LAMA4), and then MCAM-expressing CHO cells were assessed for their ability to bind to 650-labeled laminin 411 as described in the Materials and Methods. “No 411” and “411” control conditions marked 100% and 0% blocking activity, respectively. Fluorescent signal was assessed via flow cytometric analyses and plotted as mean fluorescence intensity (MFI). All 19C12 variants retained their MCAM blocking and LAMA4 binding activity when compared with chimeric 19C12.

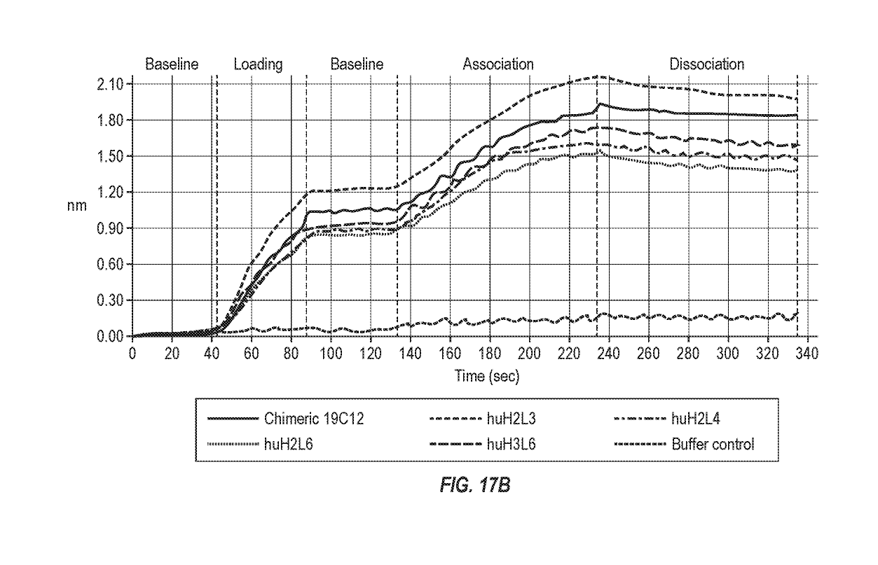

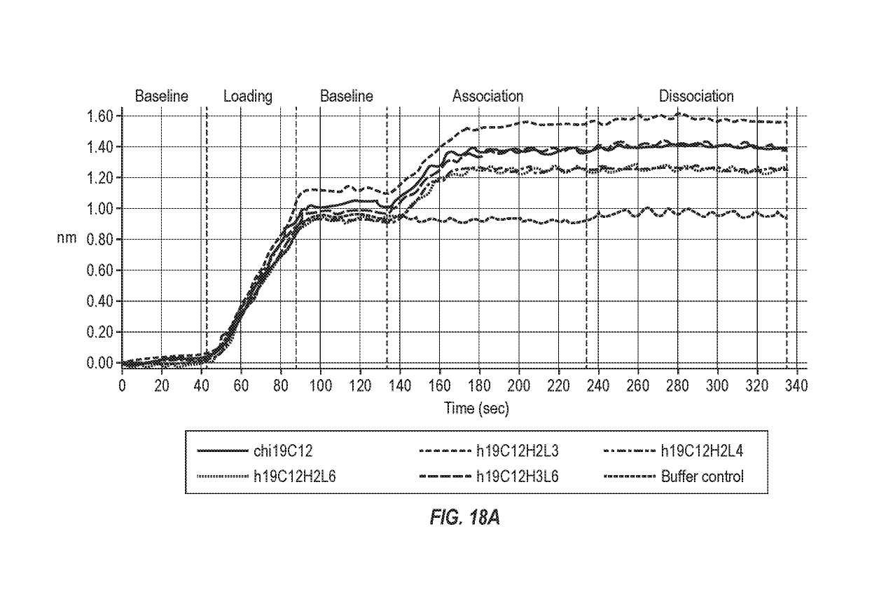

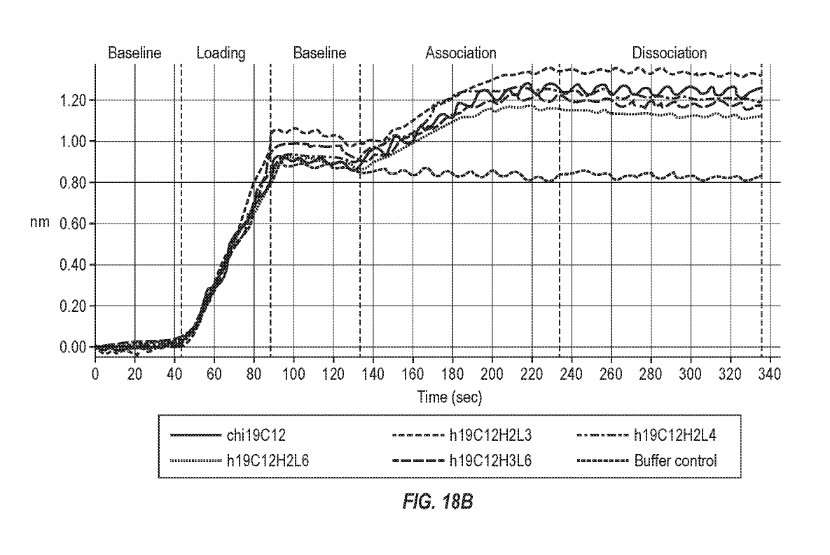

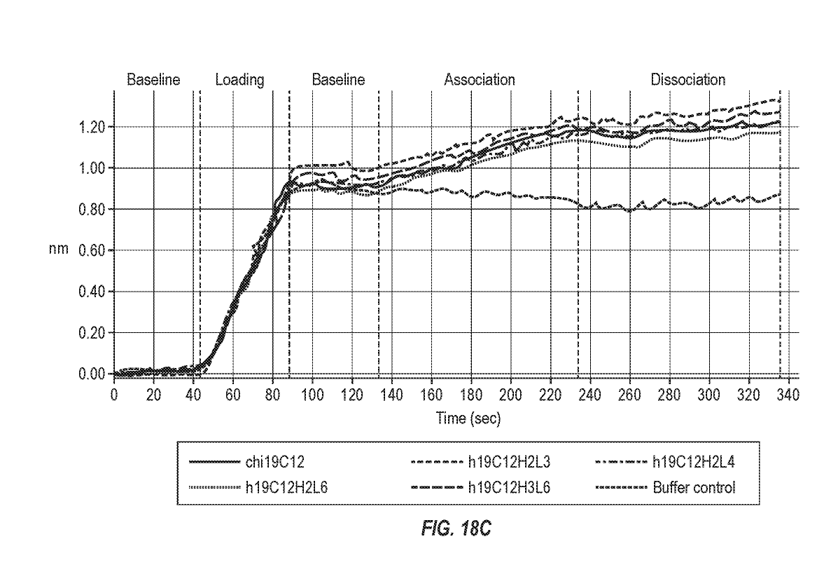

Relative binding and on/off rates were analyzed by ForteBio. In FIG. 17A, the anti-His sensor was loaded with 10 ug/ml of purified His-LAMA4 followed by loading of 10 ug/ml of chimeric 19C12, H2L3, H2L4, H2L6, and H3L6. Association and dissociation were analyzed. In FIG. 17B, the goat anti-human Fc sensor was loaded with chimeric 19C12, H2L3, H2L4, H2L6, and H3L6 at 10 ug/ml followed by the loading of 10 ug/ml of His-LAMA4. Association and dissociation were analyzed. In FIG. 18A-C, the anti-His sensor was loaded with 10 ug/ml of LAMA-His followed by loading of the chimeric 19C12, H2L3, H2L4, H2L6, and H3L6 antibodies. The concentrations of the antibodies in FIG. 18A-C were 33.3 nM, 16.7 nM, and 8.33 nM, respectively. Association and dissociation of these antibodies were compared. Table 7 summarizes the association rate (kₒₙ), dissociation rate (k_{dis}), and binding affinity constant (K_{d}) of different antibodies detected at different concentrations by ForteBio. The association rates, dissociation rates, and binding affinity constants for the humanized variants H2L3, H2L4, H2L6, and H3L6 were all comparable to the chimeric 19C12 antibody.

TABLE 7
Association Rates, Dissociation Rates, and Binding Affinity Constants
of Chimeric and Humanzied Antibodies

| | Dissoc | Conc. | | | | | | | |
|---|---|---|---|---|---|---|---|---|---|
| Antibody | Loc. | (nM) | Response | K_{D} (M) | kₒₙ (M⁻¹s⁻¹) | kₒₙ error | k_{dis} (s⁻¹) | k_{dis} error | Rmax |
| chi19C12 | A7 | 33.3 | 1.3802 | 1.11E−10 | 4.37E+06 | 1.12E+06 | 4.87E−04 | 7.85E−04 | 1.3584 |
| chi19C12 | A7 | 16.7 | 1.2655 | 5.38E−11 | 7.49E+06 | 1.96E+06 | 4.03E−04 | 8.75E−04 | 1.2002 |
| chi19C12 | A7 | 8.33 | 1.1467 | 1.26E−11 | 2.00E+07 | 7.35E+06 | 2.51E−04 | 9.96E−04 | 1.0789 |
| H2L3 | B7 | 33.3 | 1.5413 | 1.15E−10 | 4.29E+06 | 1.08E+06 | 4.93E−04 | 7.77E−04 | 1.5217 |
| H2L3 | B7 | 16.7 | 1.3392 | 5.07E−11 | 7.16E+06 | 1.88E+06 | 3.63E−04 | 8.97E−04 | 1.2765 |
| H2L3 | B7 | 8.33 | 1.2295 | 1.24E−11 | 2.07E+07 | 7.80E+06 | 2.56E−04 | 9.94E−04 | 1.1522 |
| H2L4 | C7 | 33.3 | 1.2540 | 1.27E−10 | 4.87E+06 | 1.20E+06 | 6.20E−04 | 7.12E−04 | 1.2473 |
| H2L4 | C7 | 16.7 | 1.2584 | 2.27E−11 | 9.98E+06 | 2.57E+06 | 2.27E−04 | 7.44E−04 | 1.2226 |
| H2L4 | C7 | 8.33 | 1.1755 | 1.61E−11 | 1.67E+07 | 5.18E+06 | 2.68E−04 | 9.58E−04 | 1.0987 |
| H2L6 | D7 | 33.3 | 1.2584 | 1.23E−10 | 4.75E+06 | 1.19E+06 | 5.85E−04 | 7.35E−04 | 1.2469 |
| H2L6 | D7 | 16.7 | N/A | N/A | N/A | N/A | N/A | N/A | N/A |
| H2L6 | D7 | 8.33 | 1.1259 | 1.54E−11 | 2.00E+07 | 7.08E+06 | 3.08E−04 | 9.64E−04 | 1.0524 |
| H3L6 | E7 | 33.3 | 1.3759 | 1.13E−10 | 4.89E+06 | 1.29E+06 | 5.53E−04 | 7.57E−04 | 1.3693 |
| H3L6 | E7 | 16.7 | 1.2083 | 6.26E−11 | 8.01E+06 | 2.11E+06 | 5.01E−04 | 8.58E−04 | 1.1654 |
| H3L6 | E7 | 8.33 | 1.1975 | 1.35E−11 | 2.11E+07 | 7.97E+06 | 2.86E−04 | 9.84E−04 | 1.1127 |

Biacore full binding kinetic analysis of antibodies was then carried out. Fab fragments were generated as described in the Materials and Methods, and SPR analysis was performed as described in the Materials and Methods. Detailed binding kinetic parameters (association rate (k_{assoc}), dissociation rate (k_{dissoc}), and binding affinity constant (K_{d})) were determined for Fabs of chimeric 19C12 and humanized H2L3. Binding kinetic parameters for the humanized 19C12 variant H2L3 were comparable to those for chimeric 19C12 (see Table 8).

TABLE 8
Biacore Assay Comparing Binding
of Hu19C12 Variant H2L3 and Chimeric 19C12 to LAMA4

| Antibody | k_{assoc (M}⁻¹s⁻¹) | k_{dissoc} (s⁻¹) | K_{d} (M) |
|---|---|---|---|
| Chimeric | 9.5 × 10⁶ | 5.9 × 10⁻³ | 6.2 × 10⁻¹⁰ |
| H2L3 | 9.8 × 10⁶ | 7.7 × 10⁻³ | 7.8 × 10⁻¹⁰ |

In addition, steady-state approximations of the binding affinity constant (K_{D}) were determined for Fabs of chimeric 19C12 and humanized variants H2L3, H2L4, H2L6, and H3L6. Again, binding kinetic parameters for the humanized 19C12 variants were comparable to those for chimeric 19C12 (see Table 9).

TABLE 9
Steady-State Approximations of Binding Affinity Constants
for Hu19C12 Variants H2L3 and Chimeric 19C12

| Antibody | K_{D} (M) |
|---|---|
| Chimeric | 9.8 × 10⁻¹⁰ |
| H2L3 | 1.9 × 10⁻⁹ |
| H2L4 | 7.1 × 10⁻⁹ |
| H2L6 | 8.4 × 10⁻⁹ |
| H3L6 | 7.2 × 10⁻⁹ |

In addition, steady-state approximations of the binding affinity constant (K_{D}) were determined for humanized 19C12 variants H2L3 and H3L6 by loading humanized 19C12 IgG on the anti-human Fc sensor and analyzing binding of free laminin α4 to the bound antibody (see Table 10). Binding parameters for the H2L3 and H3L6 humanized 19C12 variants in this Biacore assay (see Table 10) were comparable to the binding parameters observed through ForteBio analysis (see Table 7).

TABLE 10
Steady-State Approximations of Binding Affinity Constants
for Hu19C12 Variants H2L3 and H3L6

| Antibody | K_{D} (M) |
|---|---|
| H2L3 | 1.86 × 10⁻⁹ |
| H3L6 | 1.36 × 10⁻⁹ |

The specific binding between the 19C12 humanized variants (19C12 chi, H2L3, H2L4, H2L6, H3L6 and isotype control) and LAMA4 was further tested by assessing the variants' ability to stain wild-type vs. LAMA4.KO mouse brains. Humanized monoclonal antibodies against LAMA4 were generated as described in the Materials and Methods. Perfused and fresh frozen WT and LAMA4.KO mouse brains were cryosectioned, acetone-fixed, and stained with LAMA4 primary antibody followed by anti-human-594 secondary antibodies. The 19C12 chimeric and humanized variants all showed specific staining of LAMA4-positive mouse brain vasculature while failing to stain LAMA4.KO tissue above background isotype antibody control levels, demonstrating that the 19C12 humanized variants all retain LAMA4-specific binding activity in tissue.

Finally, 19C12 variants were tested for aggregation resistance. Antibody samples were stored in a 37° C. incubator for 4 weeks, during which aliquots were taken out aseptically immediately before each measurement. Dynamic Light Scattering measurements were taken in a Wyatt DynaPro Nanostar Dynamic Light Scattering instrument in 10 microliter size volumes within a quartz cuvette. All measurements were obtained at 37° C., with each measurement having 10 acquisitions with an acquisition time of 5 seconds. Regularization was done by the Wyatt Technology Dynamics 7.0 software using a Rayleigh Spheres model. Minimal loss of monomeric antibody was seen up to 4 weeks of incubation at 37° C. (see Table 11). In addition, no appreciable differences were noted between the different antibody variants.

TABLE 11
19C12 Antibody Variants Analyzed by Dynamic Light Scattering

| | % Mass of | | |
|---|---|---|---|
| | Monomeric Antibody Peak | | |
| | Week 0 | | |
| Antibody | (pre-incubation) | Week 1 | Week 4 |
| Chimeric | 99.8 | 99.7 | 97.9 |
| H2L3 | 99.8 | 99.5 | 99.8 |
| H2L4 | 99.0 | 100.0 | 99.9 |
| H2L6 | 99.6 | 99.4 | 99.4 |
| H3L6 | 99.6 | 99.1 | 98.9 |

### Example 7

### Treatment with Anti-LAMA4 Antibody 19C12 Results in Slowed Melanoma

Tumor Growth In Vivo Accompanied by Morphologic Changes in LAMA4 Tumor Distribution in a Mouse Xenograft Model

SCID mice (n=10 per treatment group) were implanted subcutaneously with WM266.4 human melanoma tumor cells. Animals were dosed weekly via intraperitoneal injections with antibody (i.e., 19C12 or control antibody) at doses of 1, 10, or 30 milligrams per kilogram, and tumor volumes were measured twice per week starting at 20 days post-implantation (DPI). Dosing, formulation, and measurements were carried out by three different researchers, all of whom were blinded. 8G9 was used as a mouse IgG2b isotype control.

Vascular inflammation (vasculitis) was assessed using fresh frozen heart tissue from the antibody-treated mice and costained using CD31 and CD3 antibodies. We concluded that there was no detectable heart vasculitis due to the absence of CD3-positive T-cells in heart blood vessels.

To assess LAMA4 tumor morphology, we sectioned fresh frozen WM266.4 xenograft tumor tissue (n=2 per treatment group) from the antibody-treated mice and stained with anti-LAMA4 polyclonal antibodies. We found that 19C12-treated mice exhibited smaller and brighter LAMA4-positive structures in the tumor stromal space compared to control mice, and this effect was dose-dependent.

These data demonstrate dose-dependent inhibition of human melanoma tumor growth in a mouse xenograft model, accompanied by morphologic changes in LAMA4 tumor distribution. Combined with the cell adhesion data from Examples 4 and 9, these data suggest that the MCAM- and integrin-α6β1-binding activity of LAMA4 contributes to both tumor adhesion and growth.

### Example 8

### Anti-LAMA4 Antibody 19C12 Stains Human Patient Breast Tumor and Skin Melanoma Tissue

Fresh frozen human breast tumor microarrays (Biochain; includes three samples of healthy breast tissue) were stained with 19C12. A mouse IgG1 antibody was used as a control. The majority of breast tumors stained positive with the 19C12 antibody, whereas the mouse IgG1 control antibody failed to stain the tissue. The 19C12 antibody failed to stain healthy breast tissue.

Fresh frozen human melanoma skin tumors and lung metastases (and control healthy lung and skin tissue) were stained with 19C12 and a mouse IgG control antibody. Whereas the mouse IgG control antibody failed to stain the tissue, 19C12 was highly reactive in all tissue tested. The 19C12 signal was higher in tumor tissue compared to corresponding healthy tissue.

### Example 9

### Anti-LAMA4 Antibody 19C12 Blocks Tumor Cell Adhesion In Vitro in Several Different Tumor Types

ELISA plates were precoated overnight at 4 degrees C. with 10 ug/ml recombinant human laminin 411, washed, blocked with 1% BSA/MEM, and incubated with 20 ug/ml antibodies in 0.1% BSA/MEM for 1 hour at room temperature. Various tumor cell lines were detached from flasking using Versene, washed with 0.1% BSA/MEM, and resuspended at 300,000 cells/ml. The cell suspensions were added to the ELISA plates, allowed to adhere in the incubator for 1.5 hours at 37 degrees C., washed, stained with crystal violet, and analyzed by a microplate reader to measure the magnitude of cell adhesion. Uncoated wells, wells without cells, buffer, and mouse IgG were used as control conditions. The results showed that 19C12 blocks tumor cell adhesion in vitro in several different tumor types.

### Example 10

### Anti-Laminin Antibodies Inhibit Laminin-411-Induced pAkt Activation

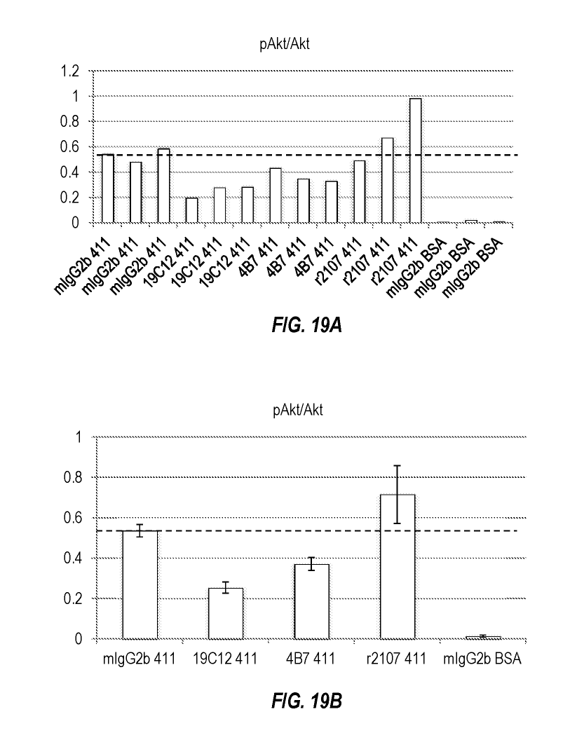

WM266.4 human tumor melanoma cells were serum-starved for 24 h and then resuspended into serum-free cell culture media with 10 ug/ml laminin 411 (LAMA4 in complex with gamma1 and beta1 chains) and 20 ug/ml 19C12, 4B7 (antibody that binds to LG4-5 modules of the G domain of laminin α4), r2107 (anti-MCAM), or mIgG2b control antibody for 30 minutes. BSA protein was used as a control for laminin 411. Cells were then spun down and lysed for immunoblot analyses. pAkt and total Akt levels were assessed by immunoblot. Ratios of these levels (pAkt/Akt) are shown in FIG. 19A & B. Each condition (mIgG2B+laminin 411; 19C12+laminin 411; 4B7+laminin 411; r2107+laminin 411; and mIgG2b +BSA) was tested in triplicate. FIG. 19A shows the results for each individual sample, and FIG. 19B shows the averages and standard errors for each condition. As shown in FIG. 19B, laminin 411 induced pAkt signaling (i.e., higher pAkt/Akt ratio) compared to BSA control, and the anti-laminin antibodies partially blocked laminin-411-induced pAkt activation (˜50% inhibition with 19C12, and ˜30% inhibition with 4B7). In contrast, r2107 (anti-MCAM) did not inhibit laminin-411-induced pAkt activation.

### Example 11

### Effects of Laminin 411 and Anti-Laminin Antibodies on Notch Signaling

Because Notch ligand D114 transcription/translation requires integrin ligation and subsequent phospho-Akt signaling, anti-LAMA4 antibodies are tested for effects on Notch signaling. HUVEC, WM266.4, and RAW cells are resuspended in cell culture media with 10 ug/ml laminin-411 (LAMA4 in complex with gamma1 and beta1 chains) and 20 ug/ml 19C12, 15F7 (antibody that binds to LG4-5 modules of the G domain of laminin α4), 4B7 (antibody that binds to LG4-5 modules of the G domain of laminin α4), r2107 (anti-MCAM), or mIgG2b control antibody for 24 hrs. BSA protein is used as a control for laminin 411. Cells are spun down and lysed for immunoblot analyses for cleaved/activated Notch1, D114, MCAM, actin, pAkt, and Akt. In addition, qPCR analysis for Hey1, MCP-1 (monocyte chemoattractant in inflammation), MCAM, LAMA4, and GAPDH is undertaken.

### Example 12

### Effects of Anti-Laminin Antibodies in In Vivo Obesity Models

Because Akt signaling is important for Notch signaling, and Notch signaling encourages growth of adipocytes, antibodies against LAMA4 are tested in in vivo obesity models for effects on weight gain/loss and adipocyte metabolism and lipolysis. High-fat diet (HFD)-driven weight gain in mice is assessed in response to anti-MCAM and laminin 411 antibodies. Wild-type C57BL/6 mice are fed a high-fat diet (e.g., rodent diet with 45% kcal % fat, such as product #D12451 from Research Diets, Inc.) ad libitum. Four experimental groups are tested: (1) mice treated with control Ig; (2) mice treated with anti-MCAM antibody (e.g., r2107); (3) mice treated with antibody that binds to LG4-5 modules of the G domain of laminin α4 (e.g., 4B7); and (4) mice treated with antibody that binds to LG1-3 modules of the G domain of laminin α4 (e.g., 19C12). There are ten mice in each group, and each mouse is treated with 10 mg/kg/week antibody for three to four months. Weight measurements are taken every two to four weeks.

To assess localization of LAMA4 to adipose tissue, anti-LAMA4 antibody (compared to isotype control antibody) is intravenously administered to mice. Staining is then undertaken to assess localization to adipose tissue.

### Example 13

### Materials and Methods

### DNA Constructs

pCMV-driven C-terminal Myc/flag-tagged cell adhesion molecule constructs were obtained from Origene (TrueORF Gold Clones: NM_000210, NM_002204, NM_002211, NM_001006946, NM_002998, NM_002999.2).

### LAMA4 Knockout Mouse

Lamaα4 null mice originally obtained from Dr. Karl Tryggvason (Karolinska University).

Generation of Recombinant MCAM-Fc Protein and hMCAM.CHO Cell Line

MCAM-Fc was generated in house by fusing the extracellular domain of human or mouse MCAM to human IgG1 and produced/purified in CHO cells using standard techniques. hMCAM.CHO cell line was generated by transfection of CHO cells with the full length human MCAM gene, selected for stable expression using neomycin and sorted for high expressers using flow cytometric sorting.

### EAE Mouse Tissue

For EAE studies, 8-16 week old SJL mice (Jackson) were immunized with PLP 139-151 peptide emulsified in CFA. Progression of disease was monitored daily and scored in a blinded fashion by standard techniques. Mice were sacrificed 35 days after PLP immunization and brains and spinal cords were analyzed for infiltration of immune cells. Brains and spinal cords were snap frozen in OCT and analyzed by fluorescent microscopy as described below.

### Antibody Generation

Recombinant mouse laminin 4 (Lama4) obtained from R&D Systems and 10 week old Lama4 null mice originally obtained from Dr. Karl Tryggvason (Karolinska University) were used to develop the antibodies. Purified laminin α4 (LAMA 4) was suspended in RIBI adjuvant at 10 μg LAMA4/25 μl adjuvant. Mice were anesthetized with isoflurane and 3 mice were immunized into each rear footpads or rear hock with 5 ug Lama 4 in RIBI adjuvant while two mice were immunized with 12.5 ug Lamα4 in RIBI adjuvant into the hock with a 27 gauge needle. Mice were injected following the above procedure on days 0, 4, 12, 16 and 20. On day 24 animals are euthanized and the popiteal and inguinal lymph nodes are removed in a sterile hood. The nodes are dissociated and fused with SP2/0 using a modification of the Kohler and Milstein protocol that incorporates Electrofusion instead of PEG fusion. Fused cells are plated into 96 well plates and allowed to grow.

When cells reach half to three quarters confluence screening begins. Briefly, Costar RIA/EIA plates were coated with rabbit ant-His tag (Anaspec #29673) at 1 ug/mL, 50 uL/well, in PBS for 1 hour. Plates were then blocked with 250ul/well of 1% BSA/PBS for 15 minutes and then removed. His-tagged Lama4 was added to the plates at 0.25 ug/mL, 50 uL/well for 1 hour, and then washed 2×. 75 uL of supernatant from fusion plates was added and incubated for 1 hour, plates were washed 2×. Goat-anti-mouse (Jackson #115-035-164) was added at 1:2000 dilution in 0.5% BSA/PBS/TBST for 1 hour, then washed 5×. Plates were developed with 5 Oul/well TMB (SurModics #TMBW24) for 5 minutes, and stopped with 15 uL 2N H2SO4, and read at 450 nm Wells with OD greater than 1.0 were selected for additional screening. Cells from wells found positive by the ELISA were grown up and frozen. Supernatants were provided for the additional screening described below. Cells from wells meeting certain criteria described below were cloned using the Clonepix FL and screened using setting recommended by the company to find single cell clones. These were expanded and the antibody purified from supernatants.

hMCAM.CHO Flow Cytometric Blocking Assay

Recombinant Laminin 411 (Biolamina; 5 ug/ml final) were preincubated with anti-LAMA4 antibodies for 15-30 min at room temperature. hMCAM.CHO cells were resuspended with EDTA and incubated with 411-Antibody mixture for 30 min at 37° C. Following two washes with FACS buffer (1% FBS in PBS), cells were resuspended with 650-conjugated anti-pan-laminin antibody (1:1000; Novus Biologicals) and incubated for 20 min at 4° C., and washed again. Cells were analyzed for pan-laminin reactivity by flow cytometry using standard procedures.

LAMA4 pDisplay Flow Cytometric Binding/Blocking Assay

Human LAMA4 G-domains 1-5 and variants were cloned into pDisplay expression construct (Life Technologies) and transiently transfected into 293 cells using standard procedures. Anti-LAMA4 antibodies were incubated with cells for 30 min at 4° C. and followed by either 10 ug/ml 650-conjugated mouse MCAM-Fc or anti-mouse-650 for 30 minutes at 4° C. Cells were analyzed for anti-laminin or mMCAM-Fc binding by flow cytometry using standard procedures.

### Mouse Brain Tissue Blocking Assay

Fresh frozen mouse brains were sectioned on a cryostat at 10 um thickness, fixed in ice-cold acetone, and blocked with 5% normal goat serum in 0.2% Triton PBS. Brain tissue was then preincubated with anti-LAMA4 antibodies, quickly washed in PBS, and recombinant 488-conjugated hMCAM-Fc (Biolamina; 1 ug/ml) was added to tissue 20 min at room temperature. Following several washes in 0.1% Triton PBS, sections were mounted in Prolong mounting media (Invitrogen).

hMCAM-Fc Capture Blocking Assay

Recombinant 2.5 ug/ml hMCAM-Fc, or Fc control (Bethyl), was used to coat 96-well plates that were initially precoated overnight with 2.5 ug/ml goat anti-human Fab (Jackson Immunoresearch) and blocked with 2% BSA +0.05% TBS-T. Following 1 hr room temperature incubation, 0.25 ug/ml recombinant mouse LAMA4-His (R&D systems) preincubated with anti-LAMA4 antibodies for 15-30 min at room temperature was added to plates for 1 hour at room temperature. Following washing steps, anti-HIS-HRP antibody (Invitrogen; 1:2000) was added for 1 hour, washed, TMB substrate (SurModics) treated, and quenched with 2 N sulfuric acid. MCAM-Fc and anti-LAMA4 antibody intravenous homing experiment

5 mg/kg MCAM-Fc, 5 mg/kg human Fc control, 10 mg/kg anti-LAMA4 polyclonal antibody (R&D systems AF3837), and 10 mg/kg goat IgG control (R&D systems AB-108-C) were intravenously injected into SFL/J mice. After 1 hr, animals were transcardially perfused with PBS and brains were dissected and snap frozen.

### LAMA4 Fragment Purification

His-tagged LAMA4 G-domain fragments were cloned by standard procedures and transiently expressed in 293 cells. Protein was purified using a nickel-NTA column.

### Fluorescence Microscopy/Standard Immunofluorescent Methods

Mouse tissue was snap frozen in OCT and sectioned at 10 uM. Sections were fixed in cold acetone and stained with anti-pan-laminin (Novus Biologicals), MCAM-Fc, anti-MCAM, anti-CD4 (Dako) or anti-LAMA4 antibodies (R&D systems).

### Transient Transfected 293T Flow Cytometric Blocking Assay

Recombinant Laminin 411 (Biolamina; 5 ug/ml final) were preincubated with anti-LAMA4 antibodies for 15-30 min at room temperature. Lipofectamine 2000 (Life Technologies) transfected cells were suspended with EDTA and incubated with 411-Antibody mixture for 30 min at 37° C. with 1 mM MnCl₂. Following two washes with FACS buffer (1% FBS in PBS), cells were resuspended with 650-conjugated anti-pan-laminin antibody (1:1000; Novus Biologicals) and incubated for 20 min at 4° C., and washed again. Cells were analyzed for pan-laminin reactivity by flow cytometry using standard procedures.

### Human Melanoma Cell Adhesion Assay

Recombinant 10 ug/ml mLAMA4 (R&D systems), was used to coat 96-well plates overnight at 4° C. Following PBS washing steps, wells were blocked with 1% BSA/MEM for 1 hr at room temperature. 20 ug/ml anti-LAMA4 antibodies in 0.1% BSA/MEM were added to plates for 1 hour at room temperature. WM-266-4 cells were resuspended with EDTA, wash and resuspended at 300,000 cells/ml in 0.1%/MEM, followed by 10 minutes in the tissue culture incubator at 37° C. with the tube cap off. Following two washes with FACS buffer (1% FBS in PBS), cells were resuspended with 650-conjugated anti-pan-laminin antibody (1:1000; Novus Biologicals) and incubated for 20 min at 4° C., and washed again. Without removing antibody solutions, add cell suspension to well and incubate uncovered in tissue culture incubator for 1.5 hrs. Following a PBS wash step, cells were stained/fixed with glutaraldehyde/crystal violet solution prior to plate reader analysis at 570 nm

### Generation of Fab Fragments

Fab fragments of all antibodies were generated using the Fab Micro Preparation kit following manufacturer's directions (Pierce). Removal of liberated Fc and verification of intact final product were monitored by SDS-PAGE, and concentration was determined using the bicinchoninic acid assay (Pierce).

### SPR Measurements of Affinity

SPR analysis was performed using a Biacore T200 to compare the binding of the different laminin antibodies. For Fab preparations, anti-6xHis antibody (GE Life Sciences) was immobilized on sensor chip C1 via amine coupling, and human His-laminin-α4, mouse His-laminin-α4 (both from R & D Systems), and an unrelated 6xHis-tagged protein (as a reaction control) were captured at a level to ensure maximum binding of 25 RU. Various concentrations of Fab preparations ranging from 300-0.41 nM were passed over the captured ligands in parallel at a flow rate of 50 ul/min in running buffer (HBS +0.05% P-20, 1 mg/mL BSA), for 240s association and varying durations of dissociation. Data were double-referenced to both an irrelevant sensor not containing His-tagged ligand, and 0 nM analyte concentration to account for the dissociation of ligand from the capture moiety. Data was then analyzed using either a heterogeneous ligand model or a global 1:1 fit.

1. A monoclonal antibody that specifically binds to an epitope within the LG1-3 modules of the G domain of laminin α4 and inhibits binding of laminin α4 to MCAM.

2. The antibody of claim 1 that binds to an epitope within LG1.

3. the antibody of claim 1 that binds to an epitope within LG2

4. The antibody of claim 1 that binds to an epitope within LG3.

5. The antibody of claim 1 that binds to an epitope to which both LG1 and LG2 contribute residues.

6. The antibody of claim 1 that binds to an epitope to which both LG2 and LG3 contribute residues.

7. The antibody of claim 1 that binds to an epitope to which both LG1 and LG3 contribute residues.

8. The antibody of claim 1 that binds to an epitope to which all of LG1, LG2, and LG3 contribute residues.

9. The antibody of claim 1 that inhibits binding of laminin α4 to integrin.

10. The antibody of claim 9, wherein the integrin is integrin α6β1.

11. The antibody of claim 1 that competes with antibody 19C12 characterized by a mature heavy chain variable region of SEQ ID NO:15 and mature light chain variable region of SEQ ID NO:16, or antibody 1C1 characterized by a mature heavy chain variable region of SEQ ID NO:25 and mature light chain variable region of SEQ ID NO:26, or antibody 5Al2 characterized by a mature heavy chain variable region of SEQ ID NO:35 or 36 and mature light chain variable region of SEQ ID NO:37, or antibody 5B5 characterized by a mature heavy chain variable region of SEQ ID NO:50 and mature light chain variable region of SEQ ID NO:51, or antibody 12D3 characterized by a mature heavy chain variable region of SEQ ID NO:60 or 61 and mature light chain variable region of SEQ ID NO:62.

12. The antibody of claim 1 that binds to the same epitope on laminin α4 as 19C12, 1C1, 5Al2, 5B5, or 12D3.

13. The antibody of claim 1 comprising three light chain CDRs and three heavy chain CDRs, wherein each CDR has at least 90% sequence identity to a corresponding CDR from the heavy and light chain variable regions of 19C12 (SEQ ID NOS:15 and 16, respectively), 1C1 (SEQ ID NOS:25 and 26, respectively), 5Al2 (SEQ ID NOS:35/36 and 37, respectively), 5B5 (SEQ ID NOS:50 and 51, respectively), or 12D3 (SEQ ID NOS:60/61 and 62, respectively).

14. The antibody of claim 1 comprising three heavy chain CDRs and three light chain CDRs of 19C12, 1C1, 5Al2, 5B5, or 12D3.

15. (canceled)

16. The antibody of claim 12 that is chimeric, humanized, veneered, or human.

17. The antibody of claim 16 that has human IgG1 kappa isotype.

18. A humanized or chimeric 19C12 antibody that specifically binds to laminin α4, wherein 19C12 is a mouse antibody characterized by a mature heavy chain variable region of SEQ ID NO:15 and a mature light chain variable region of SEQ ID NO:16.

19. The humanized antibody of claim 18 comprising a humanized heavy chain comprising three CDRs of the 19C12 heavy chain variable region (SEQ ID NO:15) and a humanized light chain comprising three CDRs of the 19C12 light chain variable region (SEQ ID NO:16).

20. The humanized antibody of claim 18 comprising a humanized mature heavy chain variable region having an amino acid sequence at least 90% identical to SEQ ID NO:81 or SEQ ID NO:82 and a humanized mature light chain variable region having an amino acid sequence at least 90% identical to SEQ ID NO:85 or SEQ ID NO:88.

21. The humanized antibody of claim 20 comprising three CDRs of the 19C12 heavy chain variable region (SEQ ID NO:15) and three CDRs of the 19C12 light chain variable region (SEQ ID NO:16).

22. The humanized antibody of claim 21, provided at least one of positions L9, L22, and L85 is occupied by A, S, and T, respectively, and at least one of positions H11, H12, H16, H27, H28, H48, H91, and H108 is occupied by L, V, A, Y, A, I, F, and T, respectively.

23. The humanized antibody of claim 22, provided positions L9, L22, and L85 are occupied by A, S, and T, respectively, and positions H11, H12, H16, H27, H28, H48, H91, and H108 are occupied by L, V, A, Y, A, I, F, and T, respectively.

24. The humanized antibody of any one of claims 21-23, provided at least one of positions L1, L49, L68, L76, L77, L78, L79, and L100 is occupied by N, C, R, D, P, V, E, and A, respectively.

25. The humanized antibody of any one of claims 21-23, provided at least one of positions H1, H20, H38, H43, and H69 is occupied by E, I, K, E, and L, respectively.

26. The humanized antibody of claim 24, provided positions L1, L49, and L68 are occupied by N, C, and R, respectively.

27. The humanized antibody of claim 24, provided position L1 is occupied by N.

28. The humanized antibody of claim 24, provided positions L1, L49, L68, L76, L77, L78, L79, and L100 are occupied by N, C, R, D, P, V, E, and A, respectively.

29. The humanized antibody of claim 24, provided positions L1, L77, L78, L79, and L100 are occupied by N, P, V, E, and A, respectively.

30. The humanized antibody of claim 24, provided position L77 is occupied by P.

31. The humanized antibody of claim 24, provided positions L77, L78, L79, and L100 are occupied by P, V, E, and A, respectively.

32. The humanized antibody of claim 25, provided positions H20, H38, H43, and H69 are occupied by I, K, E, and L, respectively.

33. The humanized antibody of claim 25, provided position H1 is occupied by E.

34. The humanized antibody of claim 20, comprising a mature heavy chain variable region having an amino acid sequence at least 95% identical to SEQ ID NO:81 or SEQ ID NO:82 and a mature light chain variable region having an amino acid sequence at least 95% identical to SEQ ID NO:85 or SEQ ID NO:88.

35. The humanized antibody of claim 20, wherein the mature heavy chain variable region has an amino acid sequence of SEQ ID NO:81 and the mature light chain variable region has an amino acid sequence of SEQ ID NO:85.

36. The humanized antibody of claim 20, wherein the mature heavy chain variable region has an amino acid sequence of SEQ ID NO:81 and the mature light chain variable region has an amino acid sequence of SEQ ID NO:86.

37. The humanized antibody of claim 20, wherein the mature heavy chain variable region has an amino acid sequence of SEQ ID NO:81 and the mature light chain variable region has an amino acid sequence of SEQ ID NO:88.

38. The humanized antibody of claim 20, wherein the mature heavy chain variable region has an amino acid sequence of SEQ ID NO:82 and the mature light chain variable region has an amino acid sequence of SEQ ID NO:88.

39. The antibody of claim 1 that is an intact antibody.

40. The antibody of claim 1 that is a single-chain antibody, Fab, or Fab′2 fragment.

41. The humanized antibody of claim 18, wherein the mature light chain variable region is fused to a light chain constant region and the mature heavy chain variable region is fused to a heavy chain constant region.

42. The humanized antibody of claim 41, wherein the heavy chain constant region is a mutant form of a natural human heavy chain constant region which has reduced binding to a Fcγ receptor relative to the natural human heavy chain constant region.

43. (canceled)

44. The humanized antibody of claim 41, wherein the mature heavy chain variable region is fused to a heavy chain constant region having the sequence of SEQ ID NO:89 and/or the mature light chain variable region is fused to a light chain constant region having the sequence of SEQ ID NO:90.

45. The humanized antibody of, claim 18 provided any differences in CDRs of the mature heavy chain variable region and mature light chain variable region from SEQ ID NOS:15 and 16, respectively reside in positions H60-H65.

46. A pharmaceutical composition comprising the antibody of claim 1 and a pharmaceutically acceptable carrier.

47. A nucleic acid encoding the heavy and/or light chain(s) of an antibody as described in claim 1.

48. The nucleic acid of claim 46 having a sequence comprising any one of SEQ ID NOS:91-92, 95-96, 99-101, 105-106, 109-111, or 115-123.

49. A recombinant expression vector comprising a nucleic acid of claim 47.

50. A host cell transformed with the recombinant expression vector of claim 49.

51. A method of humanizing an antibody, the method comprising:
(a) determining the sequences of the heavy and light chain variable regions of a mouse antibody;
(b) synthesizing a nucleic acid encoding a humanized heavy chain comprising CDRs of the mouse antibody heavy chain and a nucleic acid encoding a humanized light chain comprising CDRs of the mouse antibody light chain;
(c) expressing the nucleic acids in a host cell to produce a humanized antibody;
wherein the mouse antibody is 19C12, 1C1, 5Al2, 5B5, or 12D3.

52. A method of producing a humanized, chimeric, or veneered antibody, the method comprising:
(a) culturing cells transformed with nucleic acids encoding the heavy and light chains of the antibody, so that the cells secrete the antibody; and
(b) purifying the antibody from cell culture media;
wherein the antibody is a humanized, chimeric, or veneered form of 19C12, 1C1, 5Al2, 5B5, or 12D3.

53. A method of producing a cell line producing a humanized, chimeric, or veneered antibody, the method comprising:
(a) introducing a vector encoding heavy and light chains of an antibody and a selectable marker into cells;
(b) propagating the cells under conditions to select for cells having increased copy number of the vector;
(c) isolating single cells from the selected cells; and
(d) banking cells cloned from a single cell selected based on yield of antibody;
wherein the antibody is a humanized, chimeric, or veneered form of 19C12, 1C1, 5Al2, 5B5, or 12D3.

54. The method of claim 53, further comprising propagating the cells under selective conditions and screening for cell lines naturally expressing and secreting at least 100 mg/L/10⁶ cells/24 h.

55. A method of suppressing an undesired immune response in a patient, the method comprising administering to the patient an effective regime of the antibody of claim 1.

56-64. (canceled)

65. A method of treating or effecting prophylaxis of a cancer in a patient having or at risk for the cancer, the method comprising administering to the patient an effective regime of the antibody of claim 1.

66-67. (canceled)

68. A method of inhibiting binding of laminin α4 to MCAM in a biological sample, the method comprising contacting the biological sample with an effective amount of the antibody of claim 1.

69. A method if inhibiting binding of laminin α4 to integrin α6β1 in a biological sample, the method comprising contacting the biological sample with an effective amount of the antibody of claim 1.

70. A method of inhibiting cell adhesion in a biological sample, the method comprising contacting the biological sample with an effective amount of the antibody of claim 1.

71-72. (canceled)

73. A method of inhibiting angiogenesis in a patient, the method comprising administering to the patient an effective regime of the antibody of claim 1.

74. (canceled)

75. The antibody of claim 1 that competes with antibody 19C12 characterized by a mature heavy chain variable region of SEQ ID NO:15 and mature light chain variable region of SEQ ID NO:16, or antibody 1C1 characterized by a mature heavy chain variable region of SEQ ID NO:25 or 141 and mature light chain variable region of SEQ ID NO:26, or antibody 5Al2 characterized by a mature heavy chain variable region of SEQ ID NO:35 and mature light chain variable region of SEQ ID NO:37, or antibody 5B5 characterized by a mature heavy chain variable region of SEQ ID NO:50 and mature light chain variable region of SEQ ID NO:51, or antibody 12D3 characterized by a mature heavy chain variable region of SEQ ID NO:60 or 61 and mature light chain variable region of SEQ ID NO:62.

76. The antibody of claim 1 comprising three light chain CDRs and three heavy chain CDRs, wherein each CDR has at least 90% sequence identity to a corresponding CDR from the heavy and light chain variable regions of 19C12 (SEQ ID NOS:15 and 16, respectively), 1C1 (SEQ ID NOS:25/141 and 26, respectively), 5Al2 (SEQ ID NOS:35 and 37, respectively), 5B5 (SEQ ID NOS:50 and 51, respectively), or 12D3 (SEQ ID NOS:60/61 and 62, respectively).

77. The humanized antibody of claim 41, wherein the mature heavy chain variable region is fused to a heavy chain constant region having the sequence of SEQ ID NO:89, 138, or 150 and/or the mature light chain variable region is fused to a light chain constant region having the sequence of SEQ ID NO:90 or 139.

78. The nucleic acid of claim 46 having a sequence comprising any one of SEQ ID NOS:91-92, 95-96, 99, 101, 105-106, 109-111, 115-123, 146, 148, 149, or 151.

79. A method of treating or effecting prophylaxis of obesity or an obesity-related disease in a patient having or at risk for obesity or the obesity-related disease, the method comprising administering to the patient an effective regime of the antibody of claim 1.

80. (canceled)
